(19) United States
(12) Patent Application Publication
(10) Pub. No.: US 2025/0032405 A1
(43) Pub. Date: Jan. 30, 2025

(54) HYDROGEL FORMULATIONS, VACCINES, AND METHODS OF USE THEREOF

(71) Applicant: CELLSREGEN INC., NEWTON, MA (US)

(72) Inventors: David R. ELMALEH, Newton, MA (US); Aram AKBARZADEH, Cambridge, MA (US); Minh Khanh NGUYEN, Winston-Salem, NC (US)

(73) Assignee: CELLSREGEN INC., NEWTON, MA (US)

(21) Appl. No.: 18/696,894

(22) PCT Filed: Sep. 28, 2022
(86) PCT No.: PCT/US22/45019

§ 371 Date: Mar. 28, 2024

Related U.S. Application Data
(60) Provisional application No. 63/249,241, filed on Sep. 28, 2021.

(51) Int. Cl. A61K 9/00; A61K 9/06; A61K 35/28; A61K 39/00; A61K 47/42

(52) U.S. Cl. CPC A61K 9/0024; A61K 9/06; A61K 35/28; A61K 39/4644; A61K 47/42; A61K 2239/31; A61K 2239/39

(57) ABSTRACT

This invention relates to producing vaccines, such as cancer vaccines, by methods of forming an expansion of a lymphoid network in a subject the method comprising: administering to the subject a polymer suspension composition comprising: a natural polymer and cells; gelling the polymer suspension composition in vivo to form a hydrogel composition scaffold; and expanding the cell population to form an expansion of the lymphoid network, including in vivo. It also relates to hydrogel compositions comprising a natural polymer, along with cells, a pharmaceutical composition or a pharmaceutically acceptable salt thereof, a modulating agent, and/or a targeting agent, as well as methods of making and using the hydrogel compositions as biotherapeutic scaffolds for treatment of cancers or tumors, including in vivo.

### CROSS-REFERENCE TO RELATED APPLICATION

This application claims priority to U.S. Provisional Patent Application No. 63/249,241, filed Sep. 28, 2021, which is incorporated by reference herein in its entirety.

### FIELD OF INTEREST

This invention relates to methods of forming an expansion of a lymphoid network in a subject the method comprising: administering to the subject a polymer suspension composition comprising: a natural polymer and cells; gelling the polymer suspension composition in vivo to form a hydrogel composition scaffold; and expanding the cell population to form an expansion of the lymphoid network, including in vivo. It also relates to hydrogel compositions comprising a natural polymer, along with cells, a pharmaceutical composition, or a pharmaceutically acceptable salt thereof, a modulating agent, and/or a targeting agent, as well as methods of making and using the hydrogel compositions as biotherapeutic scaffolds for slow drug release of treatment of cancers or tumors, including in vivo.

### BACKGROUND

Chemotherapy, radiation, and surgery are the traditional tools in the fight against cancer. These treatments are based on destroying cancer cells by burning (radiation), poisoning (chemotherapy), or removing (surgery) the cancer cells. Although they can effectively kill cancer cells, these traditional treatments are nonspecific, resulting in damage to normal cells. In addition, many agents lose their utility as effective cancer therapies due to their systemic toxicity. With the development of cancer immunotherapy, the way of action has shifted from treating the disease site to treating the specific tumor biologic characteristics, mostly following chemotherapy, radiation or their combination. However, despite a century of discovery and development of new drugs within the immunotherapy field, current formulations leave drugs incapable of localizing at the sites of interest. In addition, current biological delivery methods are often hampered by immunogenicity, causing significant side effects affecting quality of life. These undesirable adverse events necessitate dose reductions or discontinuation of treatment resulting in a significantly poorer outcome and hampering therapeutic development. Moreover, current chemo- and biotherapeutic delivery methods are limited in targeting multiple tumors- and cell types. A major challenge in cancer metastasis is spread to other organs.

### SUMMARY

In some aspects, disclosed herein is a method using a hydrogel vaccine to form an expansion of a lymphoid network in a subject in need thereof, the method comprising: (a) administering to the subject a thermo-responsive polymer suspension composition comprising: a natural polymer, the natural polymer comprising collagen and one or more cells; (b) gelling the polymer suspension composition in vivo to form a hydrogel composition scaffold; and (c) expanding the population of the one or more cells to form an expansion of the lymphoid network.

In some aspects, disclosed herein is a thermo-responsive hydrogel vaccine comprising a thermo-responsive polymer composition, the thermo-responsive polymer suspension composition comprising: (a) a natural polymer, the natural polymer comprising collagen; and (b) one or more cells.

In some aspects, disclosed herein is a method of making a thermo-responsive hydrogel vaccine, the method comprising: (a) preparing, at a temperature at or below about 4C (4° C.), a collagen polymer suspension in a physiologically acceptable buffer; (b) mixing one or more cells into the collagen polymer suspension; and (c) placing the collagen suspension into an environment having a temperature of about 35C (35° C.) to about 41C (41° C.) for gelation of hydrogel composition.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIGS. 1A-1B are schematics. FIG. 1A is a schematic depicting exemplary immune interactions for the development of hydrogel-based vaccine formulations using a combination therapeutic/biotherapeutic-loaded hydrogel vaccine for the treatment of cancer cells in vivo. A dendritic cell (DC) activates a cytotoxic T-cell and cytotoxic T-lymphocyte-associated protein 4 (CTLA-4) antibody (top right). A T-cell with programmed cell death protein 1 (PD-1) antibodies and T-cell receptors (TCR) interacts with a tumor cell having major histocompatibility complex 2 (MHC2), programmed death ligand 1 (PD-L1) antibody, and programmed death ligand 2 (PD-L2) antibody (top center). The TCR on the CD8+ cytotoxic T-cell recognizes the major histocompatibility complex 1 (MHC1) on the cancer cell (top left), and the CD8+ cytotoxic T-cell secretes interferon-gamma (IFN-gamma, IFN-γ), tumor necrosis factor-alpha (TNF-alpha, TNF-α), granzyme B (GzmB, GrB), and the pore-forming protein perforin (PFN). PFN facilitates PFN facilitates the entry of granzymes into cells. The CD8+ cytotoxic T-cell kills the cancer cell (top left). A cluster of differentiation 4+ (CD4+) helper T-cell with TCR, CD80, CTLA-4 antibody, and PD-1 antibody interacts with a B-cell having MHC2, CD28, CD80, and PD-L1 antibody (bottom right), resulting in T-cell activation/differentiation (bottom below) and B-cell activation (bottom center) to yield memory B-cells and plasma cells (bottom left). Therefore, cancer cells are attacked overall by cytotoxic CD8+ T-cells, helper CD4+ T-cells, memory B-cells, dendritic cells, and antibodies from plasma cells (left). FIG. 1B is a schematic depicting an exemplary method of using a therapeutic/biotherapeutic-loaded, cell-based hydrogel vaccine for the treatment of a tumor in vivo. FIG. 1B demonstrates subcutaneous injection into a subject of a suspension comprising a polymer, cells, cytokines, chemokines (chemokine I and chemokine II), and antibodies (Ab) (anti-programmed cell death-1 antibody [anti-PD-1/α-PD-1 antibody] and anti-cytotoxic T-lymphocyte-associated protein 4 [anti-CTLA-4/α-CTLA-4 antibody]) (left). Subcutaneous in situ-formation of the biomaterial scaffold (top center) takes place in peripheral tissue in the tumor microenvironment (TME), allowing recruitment of immature and mature dendritic cells (DCs) by the chemokines and release of the anti-PD-1 and anti-CTLA-4 antibodies while the cells growth and proliferate to develop (dev.) high endothelial venules where the chemokines recruit B-cells and T-cells (right). The anti-PD-1 and anti-CTLA-4 antibodies, dendritic cells, B-cells, and T-cells target the tumor cells (bottom center).

FIGS. 2A-2C depict an overview of methods for designing hydrogels, as described herein. FIG. 2A is a flow chart depicting a method for designing the hydrogel with stromal lymphoid cells, as described herein, including evaluation of in vivo gelation of a thermosensitive hydrogel, optimization of 3D bioprinting parameters, assessment of cell viability through a Live/Dead assay, and assessment of drug release. FIG. 2B shows images of an exemplary combined thermosensitive hydrogel and testing thereof: (1) a gross photographic image of the hydrogel in vitro in an incubator at Day 3 (panel 1, upper left); electron micrograph of results of viability testing of dispersed cells by Live/Dead staining (Live=green; Dead=red) showing a high cell viability and cell spreading in a hydrogel construct in which the cells were encapsulated (panel 2, upper right); photograph of appearance of the hydrogel following subcutaneous implanting in vivo (panel 3, lower left); and a cumulative drug release graph demonstrating release of the drug in a consistent and sustained mode as it gradually degrades in vitro (panel 4, lower right). FIG. 2C is a photograph of thermo-responsive gels comprising 3 mg/mL (left) and 4.5 mg/mL (right).

FIGS. 3A-3C are fluorescent images depicting the in vitro proliferation and spread of HEK293 cells (human embryonic kidney cells) and mouse fibroblastic reticular cells (FRCs) embedded within collagen type I hydrogels, as shown with LiveDead staining, namely, viable cells stained green (calcein [fluorexon, fluorescein complex]), whereas dead cells stained red (ethidium homodimer-1). FIG. 3A is a series of fluorescent images depicting the proliferation and spread of HEK293 cells (initially 8M/mL) encapsulated into different concentrations of collagen type I (3 mg/mL [left], 4 mg/mL [center], 4.5 mg/mL [right]) on Day 0 (top) and Day 12 (bottom). FIG. 3B is a series of fluorescent images depicting the proliferation and spread of fibroblast reticular cells (FRC) (initially 8M/mL) encapsulated into collagen type I (7.4 mg/mL) with the images taken on day 7 (magnified at 4× [left], 10× [center], and 20× [right]). FIG. 3C is a comparison of fluorescent images depicting the proliferation and spread of HEK293 cells (left) and FRC (right) (both initially 8M/mL) encapsulated into collagen type I (7.4 mg/mL) with the images taken on day 7 (magnified at 4× with insets (upper right of each) magnified at 10×.

FIG. 4 is a graph depicting the results of a sustained release study of a model drug, fluorescein isothiocyanate (FITC) fluorescently tagged mouse immunoglobulin G (IgG), from collagen type I (4.5 mg/mL, pH 7.2-7.6) into 1× phosphate buffered saline (PBS), used as a release medium, at 37C (37° C.) in vitro. At predetermined timepoints, PBS in each microcentrifuge tube was removed and another fresh 1 ml PBS was added to the tube. The predetermined timepoints were 3 h, 6 h, 12 h, 1 d, 2 d, 3 d, 5 d, 7 d, 10 d, 14 d, 21 d, 28 d (h=hour(s); d=day(s)). Collagen type I gel without IgG was used for controls. FIG. 4 shows the cumulative percentage of fluorescently tagged IgG release as a function of time.

FIG. 5 is a series of groups of fluorescent images depicting proliferation and spread of HEK293 cells (initially 8M/mL) in different concentrations of gelatin methacryloyl (GelMA)+gelatin hydrogel compositions on Day 3 after three-dimensional (3D) bioprinting, in order to compare printing compositions with respect to cell growth in vitro as detected with LiveDead staining, namely, viable cells stained green (calcein [fluorexon, fluorescein complex]), whereas dead cells stained red (ethidium homodimer-1), as shown: G1 (8% GelMA+2% gelatin) (upper left quadrant); G2 (8% GelMA+4% gelatin) (upper right quadrant); G3 (9% GelMA+3% gelatin) (lower left quadrant); and G4 (9% GelMA+4% gelatin) (lower right quadrant). In each quadrant group of fluorescent images, the larger photo (left of each quadrant) is at 10× magnification, while the smaller photos are at 4× magnification (upper right of each quadrant) and 10× magnification (lower right of each quadrant). The greatest cell viability was G2, followed by G1, G4, and G3, in order.

FIG. 6 is a series of fluorescent images (4× magnification) depicting the proliferation and spread of FRC (initial density 5M/mL) in vitro on Day 7 after encapsulation in hydrogel compositions having different concentrations of GelMA and gelatin. After FRC encapsulation in GelMA mixed with gelatin samples maintained in a 37C incubator, the gelatin was dissolved, leaving behind the porous scaffold. Cell growth was detected with LiveDead staining, namely, viable cells stained green (calcein [fluorexon, fluorescein complex]), whereas dead cells stained red (ethidium homodimer-1), as shown (left to right): 9% GelMA+2% gelatin; 8% GelMA only; 8% GelMA+2% gelatin; and 8% GelMA+4% gelatin. The scaffolds exhibited highly porous and had better cell adhesion and growth than the neat GelMA scaffold. The greatest cell viability was G3, followed by G2 and G1, in order.

FIG. 7 is a series of fluorescent images depicting proliferation and spread of FRC cells (initial density 8M/mL) encapsulated in hydrogel compositions having different concentrations of GelMA+gelatin following 3D printing. Cell growth in vitro was detected with LiveDead staining, namely, viable cells stained green (calcein [fluorexon, fluorescein complex]), whereas dead cells stained red (ethidium homodimer-1). Images were taken of G1 (8% GelMA+4% gelatin) (group of four images on left) and G2 (7% GelMA+3% gelatin) (group of four images on right) at 4× magnification (upper and lower left of each group) and at 10× magnification (upper and lower right of each group) on Day 1 (top row in each group) and Day 3 (bottom row in each group). G2 displayed greater cell viability than G1.

FIGS. 8A-8C is a series of microscope images depicting the impact of lymphotoxin-alpha1-beta2 (LT-alpha1-beta2, LTα1β2) on FRC spreading. FIG. 8A depicts a series of 8% GelMA+4% gelatin bulk hydrogel compositions comprising 10M/mL fibroblast reticular cells (FRC) showing spreading in vitro at Day 3 after the addition of various concentrations of lymphotoxin-alpha1-beta2 (LT-alpha1-beta2, LTα1β2) (left to right): Control (no LT-alpha1-beta2), 50 ng/ml LT-alpha1-beta2, 100 ng/ml LT-alpha1-beta2, 150 ng/ml LT-alpha1-beta2, 500 ng/mL LT-alpha1-beta2. Images in the top row show 4× magnification; images in the bottom row show 10× magnification. FIGS. 8B-8C are fluorescent images depicting the spread of FRC in vitro on Day 7 after the addition of different concentrations of LT-alpha1-beta2 in 8% GelMA+4% gelatin bulk hydrogels having an initial density of 8M/mL FRC. Cell growth was detected with LiveDead staining, namely, viable cells stained green (calcein [fluorexon, fluorescein complex]), whereas dead cells stained red (ethidium homodimer-1). FIG. 8B shows the proliferation and spread of FRC on Day 7 using the following concentrations of LT-alpha1-beta2 (left to right): Control (no LT-alpha1-beta2), 50 ng/mL LT-alpha1-beta2, 100 ng/ml LT-alpha1-beta2, 150 ng/ml LT-alpha1-beta2, 500 ng/mL LT-alpha1-beta2. Results demonstrated that FRCs spread and formed a significant network at 500 ng/ml LT-alpha1-beta2. FIG. 8C shows LT-alpha1-beta2 (4×) depicting the spread of FRC on Day 7 after the addition of different concentrations of LT-alpha1-beta2 in 8% GelMA+4% gelatin bulk hydrogels (left to right): 150 ng/mL LT-alpha1-beta2, 500 ng/mL LT-alpha1-beta2. Results demonstrated that LT-alpha1-beta2 induced FRCs spreading and network formation.

FIGS. 9A-9B are graphs depicting sustained release of fluorescently-tagged mouse immunoglobulin G (IgG) from collagen type I hydrogel compositions into 1× phosphate buffered saline (PBS), used as a release medium, at 37C (37° C.) in vitro. Fluorescein isothiocyanate (FITC)-tagged mouse IgG (molecular weight [Mw] 150 kilodaltons [kDa]) was used as a model drug for anti-PD-1 antibody (also 150 kDa Mw) (see BIOXCELL™ BE0101; https://bxcell.com/product/m-pdl-1/). Compositions of 8% GelMA+4% gelatin and (FIG. 9A) and 7% GelMA+3% gelatin (FIG. 9B) were both prepared with 20 micrograms (μg, ug) FITC-tagged IgG per 100 microliters (μL, uL) composition. Crosslinking of the hydrogels was performed using the blue light on the printer for 30 sec (80% intensity, distance from light to samples was ˜6 mm), and 1 mL 1×PBS was added as the release buffer. The IgG hydrogel compositions with 1×PBS were incubated at 37C in the dark to avoid photobleaching of the FITC tag. At predetermined timepoints, PBS in each microcentrifuge tube was removed and another fresh 1 ml PBS was added to the tube. The predetermined timepoints were 3 h, 6 h, 12 h, 1 d, 2 d, 3 d, 5 d, 7 d, 10 d, 14 d, 21 d, 28 d (h=hour(s); d=day(s)). Collagen type I gel without IgG was used for controls. FIG. 9A shows the percentage of fluorescently tagged IgG release as a function of time from a hydrogel composition of 8% GelMA+4% gelatin. FIG. 9B shows the percentage of fluorescently tagged IgG release as a function of time from a hydrogel composition of 7% GelMA+3% gelatin.

FIG. 10 shows groups of fluorescent images depicting the in vitro proliferation and spread of FRC encapsulated into a hydrogel composition (8% GelMA+4% gelatin) at an initial cell density of 10 M/mL. Cells were grown for 7 days. Cell growth was detected with LiveDead staining, namely, viable cells stained green (calcein [fluorexon, fluorescein complex]), whereas dead cells stained red (ethidium homodimer-1). The images were taken at Day 3 (left set of panels) and Day 7 (right set of panels). The larger panel of each set is a view of combined 10× images, while the three smaller panels of each set each depict a 10× magnification of the large panel. After 7 days of culture, stromal networks formed through branching and joining with other adjacent cell populations.

FIG. 11 is a series of photographs depicting hydrogel vaccine administration in a subject. Fifty-five (55) C57BL/6 female mice were injected with B16F10 melanoma cells. When the cells formed a palpable tumor (top), the tumor was measured by a caliper in two dimensions in millimeter (mm), and its volume was then calculated according to the following formula: Tumor Volume (mm3)=L (Length)×W2 (W², Width²)×0.5236. Tumors reached the expected size on Day 6 (approximately 50 mm3 [mm3]). The mice were randomly assigned into one of six groups (n=6-8). Each group received a hydrogel vaccine regimen in pre-determined dose(s) and schedule(s). The hydrogel vaccine was injected subcutaneously near the palpable tumor (bottom).

FIG. 12 is a series of photographs depicting lymphoid tissue formation in vivo. A C57BL/6 female mouse is shaved in preparation for treatment (left), followed by subcutaneous injection of 4.5 mg/mL collagen type I solution. At one hour post-injection, the subcutaneous hydrogel formed is examined and measured (top right). At one week post-injection, the hydrogel still remains at the injection site indicating the hydrogel stability in mice (bottom right).

FIG. 13 is a graph depicting efficacy of the hydrogel stromal scaffold as combination therapy with nivolumab and ipilimumab with respect to tumor volume (mm³ [mm3]) over time (days after initial treatment). C57BL/6 inbred female mice were transfected with mouse B16F10 melanoma cells. Three sets of mice were treated as follows: Seven (7) mice were injected with Gel Atr001 (comprising anti-mouse PD-1 antibody [300 micrograms/mouse]+anti-mouse CTLA-4 antibody [300 micrograms/mouse]) on Day 6. Seven (7) mice were injected with Gel Atr002 (lymphoid stromal cell+chemokine+cytokine) injected subcutaneously on Day 6. Seven (7) mice were injected with Gel Atr003 (comprising anti-mouse PD-1 antibody (300 micrograms/mouse)+anti-mouse CTLA-4 antibody (300 micrograms/mouse)+stomal cell lymphoid+chemokine+cytokine) at Day 6. As a negative control, 7 mice were injected with one dose of vehicle (saline) on Day 6. As positive controls, seven (7) mice received one dose of a combination of anti-mouse PD-1 antibody (100 micrograms/mouse)+anti-mouse CTLA-4 antibody (100 micrograms/mouse) injected at Day 6, Day 9, and Day 12, while eight (8) mice received a single dose of a combination of anti-mouse PD-1 antibody (300 micrograms/mouse)+anti-mouse CTLA-4 antibody (300 micrograms/mouse) injected at Day 6.

FIG. 14 is a graph further depicting efficacy of the hydrogel stromal scaffold as combination therapy with nivolumab and ipilimumab with respect to survival rate (percent survival; %) over time (days after cell inoculation [d]). C57BL/6 inbred female mice were transfected with mouse B16F10 melanoma cells. Three sets of mice were treated as follows: Seven (7) mice were injected with Gel Atr001 (comprising anti-mouse PD-1 antibody [300 micrograms/mouse]+anti-mouse CTLA-4 antibody [300 micrograms/mouse]) on Day 6. Seven (7) mice were injected with Gel Atr002 (lymphoid stromal cell+chemokine+cytokine) injected subcutaneously on Day 6. Seven (7) mice were injected with Gel Atr003 (comprising anti-mouse PD-1 antibody (300 micrograms/mouse)+anti-mouse CTLA-4 antibody (300 micrograms/mouse)+stomal cell lymphoid+chemokine+cytokine) at Day 6. As a negative control, 7 mice were injected with one dose of vehicle (saline) on Day 6. As positive controls, seven (7) mice received one dose of a combination of anti-mouse PD-1 antibody (100 micrograms/mouse)+anti-mouse CTLA-4 antibody (100 micrograms/mouse) injected at Day 6, Day 9, and Day 12, while eight (8) mice received a single dose of a combination of anti-mouse PD-1 antibody (300 micrograms/mouse)+anti-mouse CTLA-4 antibody (300 micrograms/mouse) injected at Day 6.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth to provide a thorough understanding of the invention. However, it will be understood by those skilled in the art that the present invention may be practiced without these specific details. In other instances, well-known methods, procedures, and components have not been described in detail so as not to obscure the present invention.

The lymphatic network is a critical mechanism to cancer and metastases spread.

It would be desirable to have compounds and methods for making and using a hydrogel composition comprising a hydrogel comprising a cell and/or a pharmaceutical composition (e.g., a chemokine, a cytokine, an antibody or fragment thereof, a drug or pro-drug, a protein, a nucleic acid). It would be desirable to have methods of expanding the lymphatic network in a subject comprising a step of administering the hydrogel composition to the subject. It would also be desirable to have methods of treating, inhibiting, ameliorating, or alleviating a cancer or a tumor in a subject or a growth or a metastasis thereof, comprising a step of administering the hydrogel composition to the subject.

Disclosed herein are hydrogel compositions, methods of making hydrogel compositions, and methods of use thereof.

Disclosed herein is a hydrogel composition comprising a hydrogel formulation with proteins, cytokines, chemokines, antibodies or their fragments, nucleic acid, mRNA and drug payloads, as well as specific hydrogel formulation comprising cells for expanding stromal lymphoid networks in-vitro and in-vivo alone or in combination with one or more of the components comprising proteins, cytokines, chemokines, antibodies or their fragments, nucleic acid, siRNA. mRNA and drug payloads. In other embodiment these hydrogel formulations are designed to provide slow cancer treatment. In another aspect these formulations provide a cancer vaccine treatment. Hydrogel cell formulations comprising a pharmaceutical composition; a cytokine or a chemokine; a modulating agent; and/or a targeting agent.

Disclosed herein is a hydrogel composition for treating, inhibiting, ameliorating, or alleviating a cancer or a tumor in a subject or a growth or a metastasis thereof. Disclosed herein is a hydrogel composition for treating, inhibiting, ameliorating, or alleviating a cancer or a tumor in a subject in need thereof. Also disclosed herein is a hydrogel composition for reducing or inhibiting metastases infiltration in a subject in need thereof.

In some aspects, disclosed herein is an injectable hydrogel comprising cells (e.g., modified universal donor cells) for in vivo expansion of the endogenous lymphatic network. In some embodiments, this injectable hydrogel comprises a cell therapy. In some embodiments, this injectable hydrogel comprises a biologic vaccine (e.g., a delayed release or slow-release biologic vaccine). In some embodiments, the biologic vaccine enhances a subject's immunity to cancer spread and its treatment. In some other aspects, the stromal lymphoid expansion and its payload recruit T-cells, B-cells, NK-cells that expands cancer cell treatment.

The present disclosure relates to hydrogels and uses thereof, such as hydrogels comprising cells, e.g., a cell that can be formulated in a hydrogel, and/or comprising compositions. In some embodiments, the hydrogel comprises a cell. In some embodiments, the hydrogel comprises a composition (e.g., a chemokine, a cytokine, an antibody or fragment thereof, a drug or pro-drug, a protein, a nucleic acid). In some embodiments, proliferation and spread of a cell in a hydrogel can be used, e.g., as stromal lymphoid cell therapy vaccine treatments, alone or as a combination with a composition (e.g., a chemokine, a cytokine, an antibody or fragment thereof, a drug or pro-drug, a protein, a nucleic acid, siRNA, mRNA a modulating gent and other cancer treatment payloads), are useful for the treatment of diseases or abnormal physiological conditions.

In other embodiments, provided herein is a hydrogel network that is a potential slow therapeutic or biotherapeutic release for many anticancer therapies, including, but not limited to, chemotherapies, monoclonal antibody (mAb, moAb) therapies, chimeric antigen receptor T-cells (CAR-T), apoptotic therapies (e.g., with apoptotic cells, supernatants, proteins), small interfering ribonucleic acid (siRNA) therapies, micro ribonucleic acid (miRNA) therapies, antisense therapies, metabolic therapies, and/or inhibitor therapies.

In some embodiments the technology is a slow-release vaccine cancer cell therapy using specific injectable hydrogel containing cells (e.g., modified treated donor cells or universal donor cells) for in vivo expansion of the endogenous lymphatic network, thereby enhancing a patient's immunity to cancer spread and its treatment. In some embodiments, “donor treated cells” or “treated donor cells” comprises using cells taken from a subject who will receive treatment, e.g., to reduce immunogenicity-related side effects. In some embodiments, these cells are modified prior to injection.

In other embodiments, the product is an injectable hydrogel cell formulation that includes proteins such as anti-cytotoxic T-lymphocyte-associated protein 4 (CTLA-4) and/or a chemokine attracting T-cells and/or B-cells.

In other embodiments, the hydrogel formulation comprises a programmed death antibody such as an antibody to programmed cell death-1 (PD-1) or an antibody to programmed death ligand 1 (PD-L1), and/or other biotherapeutic agents.

Disclosed herein is a hydrogel composition, the hydrogel composition comprising a cell, a pharmaceutical composition, a cytokine or chemokine, an apoptotic protein or inhibitor of an anti-apoptotic protein, an antibody or antigen-binding domain, or a nucleic acid.

Some advantages of this technology over current cell therapy with biologics include, but are not limited to: a) immunity buildup of the protective lymphatic system; b) slow release of the cancer-targeting biotherapeutic agent; c) local intraperitoneal (IP) injection, as opposed to the present intravenous (IV) approach utilizing a bolus drug infusion that exposes all tissues in blood circulation; and/or d) slow-release, which allows for larger drug dosing.

In some aspects, disclosed herein is a method using a hydrogel vaccine to form an expansion of a lymphoid network in a subject in need thereof, the method comprising: (a) administering to the subject a thermo-responsive polymer suspension composition comprising: a natural polymer, the natural polymer comprising collagen and one or more cells; (b) gelling the polymer suspension composition in vivo to form a hydrogel composition scaffold; and (c) expanding the population of the one or more cells to form an expansion of the lymphoid network.

In some embodiments, the expansion of the lymphoid network comprises growth or development of lymphoid tissue. In some embodiments, the expansion of the lymphoid network comprises growth or development of a high endothelial vacuole (HEV).

In some embodiments, the collagen comprises gelatin methacryloyl (GelMA) or gelatin.

In some embodiments, the polymer suspension composition further comprises a pharmaceutical or biopharmaceutical composition, a modulating agent, a targeting agent, a stabilizing agent, or a combination of any of these. In some embodiments, the polymer suspension composition further comprises one or more drugs and the method further comprising controlled release of the one or more drugs. In some embodiments, the polymer suspension composition comprises a pharmaceutical or biopharmaceutical composition, a modulating agent, or combination thereof that inhibits the programmed cell death process.

In some embodiments, the polymer suspension composition further comprises a cytokine, a chemokine, or an antibody, wherein said cytokine, said chemokine, or said antibody comprises an immune checkpoint inhibitor or an anti-receptor activator of nuclear factor kappa-B ligand (anti-RANKL) agent. In some embodiments, the polymer suspension composition further comprises a chemokine, wherein said chemokine comprises chemokine (C-C motif) ligand 20 (CCL20) or chemokine (C-X-C motif) ligand 13 (CXCL13).

In some embodiments, the subject has a cancer or a tumor, and the method further comprises treating, inhibiting, ameliorating, or alleviating the cancer or the tumor or reducing or inhibiting growth, spread, or metastasis of the cancer or the tumor in the subject. In some embodiments, the hydrogel vaccine is a cancer vaccine. In some embodiments, the cancer is a melanoma, a fibrosarcoma, a lymphoma, a breast tumor, a pancreatic tumor, or a metastasis of any of these. In some embodiments, the tumor is a fibroma. In some embodiments, the administration of the polymer suspension composition comprises subcutaneous (SC) injection, intraperitoneal (IP) injection, intravenous injection (IV), intra-tumor injection, intra-lymph node injection, or tumor-adjacent injection in the subject. In some embodiments, the subject is a human or non-human mammal or a bird.

In some embodiments, the gelling of the polymer suspension composition is thermo-responsive. In some embodiments, the subject is a mammal, and gelation of the polymer suspension occurs from about 35.0C (35.0° C., 35.0 degrees C.) to about 41.0C (41.0° C., 41.0 degrees C.).

In some embodiments, the hydrogel composition has a sustained-release (SR) dosage or an extended-release (ER) dosage. In some embodiments, the SR dosage or the ER dosage extends at least about 7 days.

In some embodiments, the polymer suspension composition comprises about 1 mg/ml collagen to about 30 mg/mL collagen.

In some embodiments, the one or more cells comprise one or more stromal cells, one or more adipose cells, or one or more stem cells. In some embodiments, the one or more stromal cells comprise one or more fibroblast reticular cells (FRC), one or more follicular dendritic cells (FDC), one or more marginal reticular cells (MRC), one or more lymphatic endothelial cells (LEC), one or more high endothelial cells (HEC), one or more alpha-7 integrin pericytes (AIP), or a combination of any of these. In some embodiments, the one or more stromal cells comprise one or more stem cells, one or more differentiated stem cells, one or more modified stem cells, or one or more modified adipose cells, one or more mesenterial stem cells. In some embodiments, the one or more cells comprise one or more non-immunogenic cells. In some embodiments, the one or more cells comprise one or more cells collected from the subject in need prior to step (a).

In some embodiments, the polymer suspension composition comprises a pharmaceutical or biopharmaceutical composition, wherein the pharmaceutical or biopharmaceutical composition inhibits, ameliorates, or alleviates a cancer or growth thereof or a tumor or growth thereof or reduces or inhibits a metastasis of a cancer.

In some embodiments, the polymer suspension composition comprises a modulating agent. In some embodiments, the modulating agent comprises an immunomodulating agent. In some embodiments, the modulating agent comprises a cytokine or a chemokine, an antibody or an antigen-binding domain, a nucleic acid, or a combination of any of these. In some embodiments, the cytokine comprises lymphotoxin-alpha (LT-alpha), lymphotoxin-beta (LT-beta), lyphotoxin-alpha1-beta2 heterotrimer (LT-alpha1-beta2), lymphotoxin-alpha2-beta1 heterotrimer (LT-alpha2-beta1), or a combination of any of these. In some embodiments, the antibody or antigen-binding domain comprising a monoclonal antibody (mAb), a single chain variable fragment (scFv), a bispecific single chain variable fragment (diabody), a trispecific single chain variable fragment (triabody), a bispecific antibody, an antibody-drug conjugate (ADC), or a combination of any of these. In some embodiments, the antibody or antigen-binding domain comprises an anti-cytotoxic T-lymphocyte-associated protein 5 (anti-CTLA-4) antibody or antigen-binding domain, an anti-programmed cell death protein 1 (anti-PD-1) antibody or antigen-binding domain, or an anti-programmed death-ligand 1 (anti-PD-L1) antibody or antigen-binding domain, an anti-receptor activator of nuclear factor kappa-B ligand (anti-RANKL) antibody, or a combination of any of these. In some embodiments, the nucleic acid comprises a deoxyribonucleic acid (DNA) or a ribonucleic acid (RNA). In some embodiments, the DNA comprises an antisense DNA or a portion thereof, a complementary DNA (cDNA) or a portion thereof, or a genomic DNA or a portion thereof. In some embodiments, the RNA comprises a small interfering RNA (siRNA), microRNA, or messenger RNA (mRNA) or a portion thereof.

In some embodiments, the polymer suspension composition comprises a targeting agent. In some embodiments, the targeting agent comprises a cytokine or a chemokine, an antibody or an antigen-binding domain, a nucleic acid, or a combination of any of these. In some embodiments, the antibody or antigen-binding domain comprises a monoclonal antibody (mAb), a single chain variable fragment (scFv), a bispecific single chain variable fragment (diabody), a trispecific single chain variable fragment (triabody), a bispecific antibody, an antibody-drug conjugate (ADC), or a combination of any of these. In some embodiments, the antibody or antigen-binding domain comprises an anti-cytotoxic T-lymphocyte-associated protein 5 (anti-CTLA-4) antibody or antigen-binding domain, an anti-programmed cell death protein 1 (anti-PD-1) antibody or antigen-binding domain, or an anti-programmed death-ligand 1 (anti-PD-L1) antibody or antigen-binding domain, or a combination of any of these. In some embodiments, the nucleic acid comprising a deoxyribonucleic acid (DNA) or a ribonucleic acid (RNA). In some embodiments, the DNA comprises an antisense DNA or a portion thereof, a complementary DNA (cDNA) or a portion thereof, or a genomic DNA or a portion thereof. In some embodiments, the RNA comprises a small interfering RNA (siRNA), microRNA, or messenger RNA (mRNA) or a portion thereof.

In some embodiments, the natural polymer further comprising gelatin, fibrin, chitosan, cellulose, poly (lactic-co-glycolic acid), agarose, methylcellulose, hyaluronan, or an elastin-like polypeptide.

In some aspects, disclosed herein is a method of treating, inhibiting, ameliorating, or alleviating a cancer or a tumor or reducing or inhibiting growth or metastasis of a cancer or a tumor in a subject in need thereof, the method comprising: administering to the subject a thermo-responsive polymer suspension composition comprising: a natural polymer, the natural polymer comprising collagen; one or more cells, a pharmaceutical or biopharmaceutical composition, a modulating agent, a targeting agent, a stabilizing agent, or a combination of any of these; gelling the polymer suspension composition in vivo to form a hydrogel composition scaffold; and expanding the population of the one or more cells to form an expansion of the lymphoid network to treat, inhibit, ameliorate, or alleviate the cancer or tumor or to reduce or inhibit growth or metastasis of the cancer or the tumor in the subject.

In some aspects, disclosed herein is a method of treating, inhibiting, ameliorating, or alleviating a cancer or a tumor or reducing or inhibiting growth or metastasis of a cancer or a tumor in a subject in need thereof, the method comprising: administering to the subject a thermo-responsive polymer suspension composition comprising: a natural polymer, the natural polymer comprising collagen; and comprising one or more of cells, a pharmaceutical or biopharmaceutical composition, a modulating agent, a targeting agent, a stabilizing agent, or a combination of any of these; gelling the polymer suspension composition in vivo to form a hydrogel composition scaffold; and expanding the population of the one or more cells to form an expansion of the lymphoid network to treat, inhibit, ameliorate, or alleviate the cancer or tumor or to reduce or inhibit growth or metastasis of the cancer or the tumor in the subject.

In some aspects, disclosed herein is a method of treating an immune disease or an abnormal immune response in a subject in need thereof, the method comprising: administering to the subject a thermo-responsive polymer suspension composition comprising: a natural polymer, the natural polymer comprising collagen; and one or more cells, a pharmaceutical composition or biopharmaceutical composition, a modulating agent, a targeting agent, a stabilizing agent, or a combination of any of these; gelling the polymer suspension composition in vivo to form a hydrogel composition scaffold; and expanding the one or more cells to form an expansion of the lymphoid network to treat the immune disease or the abnormal immune response in the subject.

In some aspects, disclosed herein is a thermo-responsive hydrogel vaccine comprising a thermo-responsive polymer composition, the thermo-responsive polymer suspension composition comprising: (a) a natural polymer, the natural polymer comprising collagen; and (b) one or more cells, a pharmaceutical or biopharmaceutical composition, a modulating agent, a targeting agent, a stabilizing agent, one or more drugs, or a combination of any of these.

In some aspects, disclosed herein is a thermo-responsive hydrogel vaccine comprising a thermo-responsive polymer composition, the thermo-responsive polymer suspension composition comprising: (a) a natural polymer, the natural polymer comprising collagen; and (b) one or more cells.

In some embodiments, the thermo-responsive polymer suspension composition comprises about 1 mg/mL collagen to about 30 mg/mL collagen. In some embodiments, prior to gelation, the thermo-responsive polymer suspension composition is injectable. In some embodiments, gelation of the thermo-responsive polymer suspension composition is temperature dependent. In some embodiments, the collagen comprises gelatin methacryloyl (GelMA) or gelatin.

In some embodiments, the one or more cells comprise a stromal cell, an adipose cell, or a stem cell. In some embodiments, the stromal cell comprises a fibroblast reticular cell (FRC), a follicular dendritic cell (FDC), a marginal reticular cell (MRC), a lymphatic endothelial cell (LEC), a high endothelial cell (HEC), an alpha-7 integrin pericyte (AIP), or a combination of any of these. In some embodiments, the one or more cells comprise a non-immunogenic cell. In some embodiments, the one or more cells comprise an autologous cell.

In some embodiments, the thermo-responsive polymer suspension composition further comprises a pharmaceutical or biopharmaceutical composition, a modulating agent, a targeting agent, a stabilizing agent, one or more drugs, or a combination of any of these. In some embodiments, the thermo-responsive polymer suspension composition comprises a pharmaceutical or biopharmaceutical composition. In some embodiments, the pharmaceutical or biopharmaceutical composition inhibits, ameliorates, or alleviates a cancer or growth thereof or a tumor or growth thereof, or reduces or inhibits a growth or a metastasis of a cancer or a tumor. In some embodiments, the thermo-responsive hydrogel vaccine is a cancer vaccine.

In some embodiments, the thermo-responsive polymer suspension composition comprises a modulating agent. In some embodiments, the modulating agent comprises an immunomodulating agent. In some embodiments, the modulating agent comprises a cytokine or a chemokine, an antibody or an antigen-binding domain, a nucleic acid, a or a combination of any of these. In some embodiments, the cytokine comprises lymphotoxin-alpha (LT-alpha), lymphotoxin-beta (LT-beta), lyphotoxin-alpha1-beta2 heterotrimer (LT-alpha1-beta2), lymphotoxin-alpha2-beta1 heterotrimer (LT-alpha2-beta1), or a combination of any of these. In some embodiments, the chemokine comprising chemokine (C-C motif) ligand 20 (CCL20) or chemokine (C-X-C motif) ligand 13 (CXCL13). In some embodiments, the antibody or antigen-binding domain comprises a monoclonal antibody (mAb), a single chain variable fragment (scFv), a bispecific single chain variable fragment (diabody), a trispecific single chain variable fragment (triabody), a bispecific antibody, an antibody-drug conjugate (ADC), or a combination of any of these. In some embodiments, the antibody or antigen-binding domain comprises an anti-cytotoxic T-lymphocyte-associated protein 5 (anti-CTLA-4) antibody or antigen-binding domain, an anti-programmed cell death protein 1 (anti-PD-1) antibody or antigen-binding domain, or an anti-programmed death-ligand 1 (anti-PD-L1) antibody or antigen-binding domain, an anti-receptor activator of nuclear factor kappa-B ligand (anti-RANKL) antibody or a combination of any of these. In some embodiments, the nucleic acid comprises a deoxyribonucleic acid (DNA) or a ribonucleic acid (RNA). In some embodiments, the DNA comprises an antisense DNA or a portion thereof, a complementary DNA (cDNA) or a portion thereof, or a genomic DNA or a portion thereof. In some embodiments, the RNA comprises a small interfering RNA (siRNA), microRNA, or messenger RNA (mRNA) or a portion thereof.

In some embodiments, the thermo-responsive polymer suspension composition comprises a targeting agent. In some embodiments, the targeting agent comprises a cytokine or a chemokine, an antibody or an antigen-binding domain, a nucleic acid, or a combination of any of these. In some embodiments, the antibody or antigen-binding domain comprises a monoclonal antibody (mAb), a single chain variable fragment (scFv), a bispecific single chain variable fragment (diabody), a trispecific single chain variable fragment (triabody), a bispecific antibody, an antibody-drug conjugate (ADC), or a combination of any of these. In some embodiments, the antibody or antigen-binding domain comprises an anti-cytotoxic T-lymphocyte-associated protein 5 (anti-CTLA-4) antibody or antigen-binding domain, an anti-programmed cell death protein 1 (anti-PD-1) antibody or antigen-binding domain, or an anti-programmed death-ligand 1 (anti-PD-L1) antibody an anti-RANKL antibody or antigen-binding domain, or a combination of any of these. In some embodiments, the nucleic acid comprises a deoxyribonucleic acid (DNA) or a ribonucleic acid (RNA). In some embodiments, the DNA comprises an antisense DNA or a portion thereof, a complementary DNA (cDNA) or a portion thereof, or a genomic DNA or a portion thereof. In some embodiments, the RNA comprises a small interfering RNA (siRNA), microRNA, or messenger RNA (mRNA) or a portion thereof.

In some embodiments, the natural polymer further comprises, gelatin, fibrin, chitosan, cellulose, poly (lactic-co-glycolic acid), agarose, methylcellulose, hyaluronan, or an elastin-like polypeptide.

In some aspects, disclosed herein is a method of making a thermo-responsive hydrogel vaccine, the method comprising: (a) preparing, at a temperature at or below about 4C, a collagen polymer suspension in a physiologically acceptable buffer; (b) mixing one or more cells into the collagen polymer suspension; and (c) placing the collagen suspension into an environment having a temperature of about 35C (35° C.) to about 41C (41° C.) for gelation of hydrogel composition. In some embodiments, step (b) further comprises mixing a pharmaceutical or biopharmaceutical composition, a modulating agent, a targeting agent, a stabilizing agent, or a combination of any of these with the one or more cells into the collagen polymer suspension.

In some embodiments, the hydrogel composition further comprises a cell, a pharmaceutical composition, a modulating agent, a targeting agent, a metabolic or biochemical modulating agent, a ligand involved in modulating specific receptors, or an enzyme inhibitor.

In some embodiments, the hydrogel composition comprises a cell. In some embodiments, the cell is a non-immunogenic cell. In some embodiments, the cell is a universal donor cell. In some embodiments, the cell is a non-immunogenic universal donor cell. In some embodiments, the cell is a stromal cell, an adipose cell, a mesenterial, or a stem cell.

In some embodiments, the hydrogel comprises a scaffold or platform for maintenance, growth, or proliferation of a cell or a medium for maintenance, growth, spread, or proliferation of a cell. In some embodiments, the use of the hydrogel composition comprises the maintenance, growth, spread, or proliferation of cells formulated on or in or encapsulated within the hydrogel. In some embodiments, the maintenance, growth, spread, or proliferation of cells comprises growth or repair of a lymphatic vessel or lymph node; a blood vessel; a tissue; or an organ or a part of an organ. In some embodiments, the maintenance, growth, spread, or proliferation of cells comprises growth or repair of a lymphatic vessel; a blood vessel; a tissue; or an organ or a part of an organ. In some embodiments, the maintenance, growth, spread, or proliferation of cells comprises lymphoid network expansion. In some embodiments, the maintenance, growth, spread, or proliferation of cells comprises growth or repair of a high endothelial venule (HEV).

In some embodiments, the maintenance, growth, spread, or proliferation of cells comprises lymphoid network expansion. In some embodiments, the maintenance, growth, spread, or proliferation of cells comprises growth or repair of a high endothelial venule (HEV). In some embodiments, the composition comprises a pharmaceutical or biopharmaceutical drug, biologic or other targeting cancer treatment via biochemical, biological or chemotherapeutical mechanism of action.

In some embodiments, the modulating agent comprises an inhibitor. In some embodiments, the modulating agent comprises an activator.

In some embodiments, the modulating agent comprises an immunomodulating agent. In some embodiments, the immunomodulating agent activates an immune response. In some embodiments, the immunomodulating agent inhibits an immune response of T-cells, B-cell or natural killer cells.

In some embodiments, the modulating agent comprises a pharmaceutical composition. In some embodiments, the pharmaceutical composition comprises a therapeutic or a biotherapeutic. In some embodiments, the modulating agent comprises a cytokine or a chemokine. In some embodiments, the modulating agent comprises an apoptotic protein or an antibody or antigen-binding domain inhibiting tumor growth. In some embodiments, the modulating agent comprises a nucleic acid. In some embodiments, the modulating agent comprises a ligand, an antibody, or an antigen-binding domain.

In some embodiments, the targeting agent comprises a cytokine or chemokine. In some embodiments, the targeting agent comprises a nucleic acid. In some embodiments, the targeting agent comprises an antibody, or an antigen-binding domain.

In some embodiments, the hydrogel composition comprising a cell further comprises a composition promoting maintenance, growth, differentiation, or division of the cell. In some embodiments, the composition promoting maintenance, growth, differentiation or division of the cell comprises a cytokine or a chemokine. In some embodiments, the composition promoting maintenance, growth, differentiation or division of the cell comprises a cytokine, a chemokine, or a biotherapeutic agent.

In some embodiments, methods of using the hydrogel composition comprise expansion of the lymphatic system or network. In some embodiments, methods of using the hydrogel composition comprise expansion of the circulatory system or network. Expansion of the lymphatic system or network and/or expansion of the circulatory system or network provides a method for treating, inhibiting, ameliorating, or alleviating a disease or abnormal physiological condition in a subject. In some embodiments, the disease or abnormal physiological condition comprises a tumor or a cancer.

Disclosed herein are cell therapy treatments comprising a cell formulated on or in or encapsulated in a hydrogel.

Disclosed herein are vaccines comprising a cell formulated on or in or encapsulated in a hydrogel. In some embodiments, the vaccine comprises a biologic cell therapy vaccine.

In some embodiments, the use of the vaccine comprises the maintenance, growth, spread, or proliferation of cells formulated on or in or encapsulated within the hydrogel. In some embodiments, the maintenance, growth, spread, or proliferation of cells comprises growth or repair of a lymphatic vessel; a blood vessel; a tissue; or an organ or a part of an organ. In some embodiments, the maintenance, growth, spread, or proliferation of cells comprises lymphoid network expansion. In some embodiments, the maintenance, growth, spread, or proliferation of cells comprises growth or repair of a high endothelial venule (HEV). In some embodiments, the maintenance, growth, spread, or proliferation of cells comprises lymphoid network expansion. In some embodiments, the maintenance, growth, spread, or proliferation of cells comprises growth or repair of a high endothelial venule (HEV). In some embodiments, the polymer composition comprises a biotherapeutic agent.

In one embodiment, the vaccine comprises effective lymphatic-system network expansion for biologic cell therapy vaccine treatment alone or in combination with, e.g., other cancer modulators while masking their toxicity and targeting spread and deliver drugs to improve targeting of cancer cells.

In some embodiments, methods of using the hydrogel composition comprise treating, inhibiting, ameliorating, or alleviating a cancer or a tumor in a subject or a growth or a metastasis thereof, comprising a step of administering the hydrogel composition to the subject in a subject in need thereof. In some embodiments, methods of using the hydrogel composition comprise treating, inhibiting, ameliorating, or alleviating a cancer or a tumor in a subject in need thereof. In some embodiments, methods of using the hydrogel composition comprise treating, inhibiting, ameliorating, or alleviating metastasis of a cancer in a subject in need thereof. In some embodiments, methods of using the hydrogel composition further comprise increasing survival in the subject. In some embodiments, methods of using the hydrogel composition further comprise reducing or inhibiting metastases infiltration.

In some embodiments, the method of treating, inhibiting, ameliorating, or alleviating a tumor or cancer comprises treatment of solid tumors or cancers. In some embodiments, the method of treating, inhibiting, ameliorating, or alleviating a tumor or cancer comprises treatment of a melanoma, a fibroma, or fibrosarcoma.

In some aspects, disclosed herein is a hydrogel composition, e.g., a composition comprising a hydrogel or a hydrogel including, e.g., gelatin methacryloyl (GelMA) for bioprinting.

### Hydrogels

A “gel” is a nonfluid colloidal network or polymer network that is expanded throughout its whole volume by a fluid (e.g., a swelling agent), as defined by the International Union of Pure and Applied Chemistry (IUPAC). A gel is also a substantially dilute cross-linked system, which exhibits no flow when in the steady-state, and/or a soft, solid or solid-like material consisting of two or more components, one of which is a liquid, present in substantial quantity. A gel is mostly liquid by weight, but behaves like a solid due to its three-dimensional cross-linked network within the liquid or a dispersion of molecules of a liquid within a solid medium. “Gelation” is the process of forming a gel. A “xerogel” is an open network formed by the removal of all swelling agents from a gel. An “orangogel” is a non-crystalline, non-glassy thermo-reversible (thermoplastic) solid material composed of a liquid organic phase entrapped in a three-dimensionally cross-linked network, wherein the liquid can be, e.g., an organic solvent, mineral oil, or vegetable oil.

In some aspects, the present invention provides a hydrogel composition, i.e., a composition comprising a hydrogel.

A “hydrogel” is a gel in which the swelling agent is water. A hydrogel is a crosslinked hydrophilic polymer that does not dissolve in water. A hydrogel is a network of polymer chains that are hydrophilic, sometimes found as a colloidal gel in which water is the dispersion medium. A three-dimensional solid results from the hydrophilic polymer chains being held together by crosslinks, which prevent the network from dissolving in the high concentration of water. Hydrogels are highly absorbent (can contain over 90% water) natural or synthetic polymeric networks and possess a degree of flexibility very similar to natural tissue, due to their significant water content. (See, e.g., Caliari et al. Nature Methods 13 (5): 405-414 [incorporated by reference].)

A hydrogel comprises a crosslinked natural, synthetic, or combination hydrophilic polymer that does not dissolve significantly in water. The crosslinking may be physical and/or chemical. Physical crosslinks include, but are not limited to, hydrogen bonds, hydrophobic interactions, chain entanglements, or other types of crosslinks. A hydrogel generated via physical crosslinks may be referred to as “reversible hydrogels.” Chemical crosslinks include, but are not limited to, covalent bonds between polymer strands, and these types of hydrogels may be referred to as “permanent hydrogels.”

Hydrogels can respond to changes in environment. For example, they can be formulated to include (encapsulate) chemical systems which upon stimulation by external factors (e.g., change of temperature [thermo-responsive, thermo-sensitive, or temperature-responsive], light exposure [photo-sensitive], change of pH [pH-sensitive]) may cause specific compounds such as glucose to be liberated to the environment, in most cases by a gel-sol transition to the liquid state. Chemomechanical polymers are mostly also hydrogels, which upon stimulation change their volume and can serve as actuators or sensors.

In some embodiments, the hydrogel is a thermo-responsive hydrogel. In some embodiments, the polymer suspension composition is a thermo-responsive polymer suspension composition. In some embodiments, the hydrogel composition is a thermo-responsive hydrogel composition. In some embodiments, the hydrogel vaccine is a thermo-responsive hydrogel vaccine.

In some embodiments, the method of initiating a polymerization reaction involves the use of temperature change. For example, the polymer (e.g., fiber) composition (non-gel) is prepared at a low temperature (e.g., in an ice bucket or at 4C [4° C.]), and the polymer composition is administered to a vertebrate at the body temperature of the vertebrate, which is a temperature at which gelation occurs for formation of the hydrogel composition. In some embodiments, the polymer composition (non-gel) is prepared at a low temperature (e.g., in an ice bucket or at 4C [4°]), and the polymer composition is administered to a homeothermic vertebrate subject (a bird or a mammal), and gelation occurs at the warmer body temperature of the homeothermic vertebrate subject for formation of the hydrogel composition. In some embodiments, the polymer composition (non-gel) is prepared at a low temperature (e.g., in an ice bucket or at 4C [4° C.]), and the polymer composition is administered to a human subject, and gelation occurs at the body temperature of the human subject for formation of the hydrogel composition, e.g., at a range from about 35.0C (35.0° C.) to about 41.0C (41.0° C.), from about 35.5C (35.5° C.) to about 40.5C (40.5° C.), from about 35.5C (35.5° C.) to about 40.0C (40.0° C.), from about 35.5C (35.5° C.) to about 39.5C (39.5° C.), from about 35.5C (35.5° C.) to about 39.0C (39.0° C.), from about 35.5C (35.5° C.) to about 38.5C (38.5° C.), from about 35.5C (35.5° C.) to about 38.0C (38.0° C.), or from about 36.0C (36.0° C.) to about 37.5C (37.5° C.).

Alternatively, the polymer composition (non-gel) is prepared at a temperature higher than the body temperature of the subject, and the polymer composition is administered to the subject, and gelation occurs at the cooler body temperature of the subject for formation of the hydrogel composition. For example, the polymer composition (non-gel) is prepared at a temperature greater than body temperature of the subject, and the polymer composition is administered to a vertebrate at the body temperature of the vertebrate, which is a temperature at which gelation occurs for formation of the hydrogel composition. In some embodiments, the polymer composition (non-gel) is prepared at a warmer temperature than the body temperature of a homeothermic vertebrate subject (a bird or a mammal), and the polymer composition is administered to the homeothermic vertebrate subject, and gelation occurs at the cooler body temperature of the homeothermic vertebrate subject for formation of the hydrogel composition. In some embodiments, the polymer composition (non-gel) is prepared at a warmer temperature than the body temperature of a human subject, and the polymer composition is administered to the human subject, and gelation occurs at the body temperature of the human subject for formation of the hydrogel composition, e.g., at a range from about 35.0C (35.0° C.) to about 41.0° C. (41.0° C.), from about 35.5C (35.5° C.) to about 40.5C (40.5° C.), from about 35.5C (35.5° C.) to about 40.0C (40.0° C.), from about 35.5C (35.5° C.) to about 39.5C (39.5° C.), from about 35.5C (35.5° C.) to about 39.0C (39.0° C.), from about 35.5C (35.5° C.) to about 38.5C (38.5° C.), from about 35.5C (35.5° C.) to about 38.0C (38.0° C.), or from about 36.0C (36.0° C.) to about 37.5C (37.5° C.).

In some embodiments, the method of initiating a polymerization reaction involves the use of light as a stimulus. In this method, a “photoinitiator,” a compound that cleaves from the absorption of photons, is added to a precursor suspension which will become the hydrogel. When the precursor suspension is exposed to a concentrated source of light, the photoinitiator will cleave and form free radicals, which will begin a polymerization reaction that forms crosslinks between polymer strands.

In some embodiments, the hydrogel composition comprises a thermo-responsive hydrogel, a photo-sensitive hydrogel, or a pH-sensitive hydrogel. In some embodiments, the hydrogel composition comprises a thermo-responsive hydrogel.

Depending on their polymeric components, hydrogels can be classified as “natural hydrogels,” “synthetic hydrogels,” or “combination hydrogels” and are comprised, respectively, of natural polymers, synthetic polymers, or a combination of both natural and synthetic polymers. “Natural polymers” include, but are not limited to, collagen, gelatin, chitosan, cellulose, poly (lactic-co-glycolic acid), agarose, methylcellulose, hyaluronan, fibrin, elastin-like polypeptides, and other naturally derived polymers. A “natural hydrogel” may comprise two or more types of natural polymer (e.g., a collagen-fibrin hydrogel). “Synthetic polymers,” which include, but are not limited to, polyvinyl alcohol, polyethylene glycol, sodium polyacrylate, polyacrylamide, acrylate polymers and copolymers thereof. A “synthetic hydrogel” may comprise two or more types of synthetic polymer. A “combination hydrogel” comprises at least one natural polymer and at least one synthetic polymer.

In some embodiments, the hydrogel comprises collagen, gelatin, chitosan, cellulose, poly (lactic-co-glycolic acid), agarose, methylcellulose, hyaluronan, fibrin, or elastin-like polypeptides. In some embodiments, the hydrogel comprises collagen. In some embodiments, the hydrogel comprises collagen and chitosan, cellulose, poly (lactic-co-glycolic acid), agarose, methylcellulose, hyaluronan, or elastin-like polypeptides. In some embodiments, the hydrogel comprises collagen-fibrin. In some embodiments, the hydrogel comprises polyvinyl alcohol, polyethylene glycol, sodium polyacrylate, acrylate polymers and copolymers thereof.

Hydrogels may also comprise hybrid materials that combine elements of synthetic and natural polymers. Examples include, but are not limited to, hyaluronic acid and polypeptides.

“Nanocomposite hydrogels” are highly hydrated polymeric networks, either physically or covalently crosslinked with each other and/or with nanoparticles or nanostructures. Nanocomposite hydrogels can mimic native tissue properties, structure and microenvironment due to their hydrated and interconnected porous structure. The nanoparticles can include, but are not limited to, carbon-based, polymeric, ceramic, and metallic nanomaterials, each of which can be incorporated within the hydrogel structure to obtain nanocomposites with tailored functionality.

“Collagen” is the main structural protein in the extracellular matrix found in various connective tissues, including, but not limited to, cartilage, bones, tendons, ligaments, and skin. As the main component of connective tissue, it is the most abundant protein in mammals, making up from 25% to 35% of the whole-body protein content. Collagen is also abundant in corneas, blood vessels, the gut, intervertebral discs, and the dentin in teeth. In muscle tissue, it serves as a major component of the endomysium. Collagen constitutes one to two percent of muscle tissue and accounts for 6% of the weight of strong, tendinous muscles. Collagen comprises amino acids bound together, in some cases to form a triple helix of elongated fibril known as a collagen helix. Depending upon the degree of mineralization, collagen tissues may be rigid (bone) or compliant (tendon) or have a gradient from rigid to compliant (cartilage). The fibroblast is the most common cell that creates collagen. “Gelatin” is collagen that has been irreversibly hydrolyzed. “Gelatin methacryloyl” (“GelMA”) is an engineered gelatin-based material useful for tissue engineering, drug delivery, and 3D printing applications (Zhu et al. Nature Scientific Reports 9:6863 [https://doi.org/10.1038/s41598-019-42186-x], which is incorporated herein by reference).—There are numerous types of collagens. Fibrillar collagens include, but are not limited to Type I, Type II, Type III, Type V, and Type XI. Typically, the collagen protein is composed of a triple helix, which generally consists of two identical chains (alpha1, α1) and an additional chain that differs slightly in its chemical composition (alpha2, α2). The amino acid composition of collagen is atypical for proteins, particularly with respect to its high hydroxyproline content and, depending on the type of collagen, may have hydroxylysine content. The most common motifs in the amino acid sequence of collagen are glycine-proline-X and glycine-X-hydroxyproline, where X is any amino acid other than glycine, proline or hydroxyproline.

In some embodiments, the hydrogel composition comprises a natural polymer. In some embodiments, the hydrogel composition comprises collagen, gelatin, fibrin, alginate, chitosan, cellulose, poly (lactic-co-glycolic acid), agarose, methylcellulose, hyaluronan, and/or an elastin-like polypeptide. In some embodiments, the hydrogel composition comprises collagen. In some embodiments, the hydrogel composition comprises collagen type I. In some embodiments, the hydrogel composition comprises gelatin. In some embodiments, the hydrogel composition comprises gelatin methacryloyl (GelMA). In some embodiments, the hydrogel composition comprises collagen and gelatin, fibrin, alginate, chitosan, cellulose, poly (lactic-co-glycolic acid), agarose, methylcellulose, hyaluronan, and/or an elastin-like polypeptide. In some embodiments, the hydrogel composition comprises collagen-fibrin.

In some embodiments, the hydrogel comprises at least about 1 mg/mL collagen, at least about 2 mg/mL collagen, at least about 3 mg/mL collagen, at least about 4 mg/mL collagen at least about 5 mg/mL collagen, at least about 6 mg/mL collagen, at least about 7 mg/mL collagen, at least about 8 mg/mL collagen, at least about 9 mg/mL collagen, at least about 10 mg/mL collagen, at least about 11 mg/mL collagen, at least about 12 mg/mL collagen, at least about 13 mg/mL collagen, at least about 14 mg/mL collagen, at least about 15 mg/mL collagen, at least about 16 mg/mL collagen, at least about 17 mg/mL collagen, at least about 18 mg/mL collagen, at least about 19 mg/mL collagen, at least about 20 mg/mL collagen, at least about 21 mg/mL collagen, at least about 22 mg/mL collagen, at least about 23 mg/mL collagen, at least about 24 mg/mL collagen, at least about 25 mg/mL collagen, at least about 26 mg/mL collagen, at least about 27 mg/mL collagen, at least about 28 mg/mL collagen, at least about 29 mg/mL collagen, or at least about 30 mg/mL collagen. In some embodiments, the hydrogel comprises about 1 mg/mL collagen to about 30 mg/mL collagen.

In some embodiments, the hydrogel comprises at least about 1% collagen, at least about 1.5% collagen, at least about 2% collagen, at least about 2.5% collagen, at least about 3% collagen, at least about 4% collagen, at least about 4.5% collagen, at least about 5% collagen, at least about 5.5% collagen, at least about 6% collagen, at least about 6.5% collagen, at least about 7% collagen, at least about 7.5% collagen, at least about 8% collagen, at least about 8.5% collagen, at least about 9% collagen, at least about 9.5% collagen, at least about 10% collagen, at least about 10.5% collagen, at least about 11% collagen, at least about 11.5% collagen, at least about 12% collagen, at least about 12.5% collagen, at least about 13% collagen, at least about 13.5% collagen, or at least about 14% collagen. In some embodiments, the hydrogel comprises about 4% collagen to about 12% collagen. In some embodiments, the hydrogel comprises about 5% collagen to about 11% collagen. In some embodiments, the hydrogel comprises about 5% collagen to about 10% collagen. In some embodiments, the hydrogel comprises about 6% collagen to about 10% collagen. In some embodiments, the hydrogel comprises about 6% collagen to about 9% collagen. In some embodiments, the hydrogel comprises about 6.5% collagen to about 8.5% collagen. In some embodiments, the hydrogel comprises about 7% to about 8% collagen. In some embodiments, the density and infrastructure for lymphoid system network expansion, as well as the thermo-sensitivity adjusted for in vivo slow drug release at body temperature, the collagen component comprises from about 6% collagen to about 9% collagen. In some embodiments, the density and infrastructure for lymphoid system network expansion, as well as the thermo-sensitivity adjusted for in vivo slow drug release at body temperature, the collagen component comprises from about 7% collagen to about 8% collagen.

### The Lymphatic System

In some embodiments, methods of using the hydrogel comprise expansion of the lymphatic system or network. In some embodiments, methods of using the hydrogel comprise expansion of the circulatory system or network.

The “lymphatic system” (“lymphoid system”) is an organ system in vertebrates that is part of the circulatory system and immune system. It comprises a large network of lymph, lymphatic vessels, lymph nodes, lymphatic or lymphoid organs, and lymphoid tissues. The vessels carry lymph towards the heart.

In contrast to the cardiovascular system, the lymphatic system is not a closed system. One major function of the lymphatic system is to provide an accessory return route of plasma from the interstitial fluid to the blood.

Another major function of the lymphatic system is immune defense. Like blood plasma, lymph contains waste products and cellular debris, together with bacteria and proteins. The cells of the lymph are predominantly lymphocytes. Associated “lymphoid organs” are comprised of lymphoid tissue and are the sites either of lymphocyte production or of lymphocyte activation. The lymphoid organs also contain other types of cells, such as stromal cells, for support.

“Primary lymphoid organs” include the thymus and the bone marrow. These primary (or central) lymphoid organs generate lymphocytes from immature progenitor cells and are involved in the production and early clonal selection of lymphocyte tissues. In birds, the bursa of Fabricius is also a primary lymphoid organ.

“Bone marrow” is a semi-solid tissue found within the spongy or cancellous portions of bones. In birds and mammals, bone marrow is the primary site of new blood cell production or hematopoiesis. It is composed of hematopoietic cells, marrow adipose tissue, and supportive stromal cells. In adult humans, bone marrow is primarily located in the ribs, vertebrae, sternum, and bones of the pelvis. Bone marrow is also responsible for both the creation of T cell precursors and the production and maturation of B cells, which are important cell types of the immune system. From the bone marrow, B cells immediately join the circulatory system and travel to secondary lymphoid organs in search of pathogens. However, T cells travel from the bone marrow to the thymus, where they develop further and mature. Mature T cells then join B cells in search of pathogens. The other 95% of T cells begin a process of apoptosis, a form of programmed cell death. The bone marrow stroma contains mesenchymal stem cells (MSCs), which are also known as marrow stromal cells. These are multipotent stem cells that can differentiate into a variety of cell types. MSCs have been shown to differentiate, in vitro or in vivo, into osteoblasts, chondrocytes, myocytes, marrow adipocytes and beta-pancreatic islets cells.

The “bursa of Fabricius” is a primary lymphoid organ and epithelial organ exclusively found in birds. In birds, the bursa of Fabricius is the site of hematopoiesis and is essential for B cell development. The luminal (interior) surface of the bursa is plicated with up to 15 primary and 7 secondary plicae (folds) having hundreds of bursal follicles containing follicle-associated epithelial cells, lymphocytes, macrophages, and plasma cells. Lymphoid stem cells migrate from the fetal liver to the bursa during ontogeny. In the bursa, these stem cells acquire the characteristics of mature, immunocompetent B cells.

The “thymus” is also a primary lymphoid organ of the immune system and is the site of maturation for thymus cell lymphocytes or T cells. T cells are critical to the adaptive immune system, where the body adapts specifically to foreign invaders. The thymus is located in the upper front part of the chest, in the anterior superior mediastinum, behind the sternum, and in front of the heart. It is made up of two lobes, each consisting of a central medulla and an outer cortex, surrounded by a capsule. In response to postnatal antigen stimulation after birth, the thymus increases in size. The neonatal and pre-adolescent periods are when it is at its most active. At puberty, the thymus begins to atrophy and regress, with adipose tissue mostly replacing the thymic stroma. However, the thymus develop a severe immunodeficiency and subsequent high susceptibility to infection. In most species, the thymus consists of lobules divided by septa which are made up of epithelium, and as a result, it is often considered an epithelial organ. T cells, which provide cell-mediated immunity, mature from thymocytes, precursors of which have migrated from the bone marrow to the thymus. Thymocytes proliferate and undergo a selection process in the thymic cortex before entering the medulla to interact with epithelial cells. Thymocytes undergo a process of maturation, which involves ensuring the cells react against antigens (“positive selection”), but that they do not react against antigens found on body tissue (“negative selection”), e.g., to avoid autoimmunity. Positive selection occurs in the cortex and negative selection occurs in the medulla of the thymus. Once mature, T cells emigrate from the thymus to provide vital functions in the immune system. Further maturation occurs in the peripheral circulation.

“Secondary lymphoid organs” (SLO; “peripheral lymphoid organs”) include the spleen, the lymph nodes (which have the highest lymphocyte concentration), the tonsils, and the appendix. The secondary lymphoid organs maintain mature naïve lymphocytes and initiate an adaptive immune response and are the sites of lymphocyte activation by antigens. Activation leads to clonal expansion and affinity maturation. Mature lymphocytes recirculate between the blood and the peripheral lymphoid organs until they encounter their specific antigen.

The “spleen,” which is found in all vertebrates, acts primarily as a blood filter. The spleen plays important roles in red blood cells (erythrocytes) and the immune system. With respect to the former, it removes old red blood cells, holds a reserve of blood, and recycles iron. As a part of the mononuclear phagocyte system, it metabolizes hemoglobin removed from senescent red blood cells (erythrocytes), degrading the globin portion of hemoglobin to its constitutive amino acids and metabolizing the heme portion to bilirubin, which is removed in the liver. With respect to its role in the immune system, the spleen synthesizes antibodies in its white pulp and removes antibody-coated bacteria and antibody-coated blood cells by way of blood and lymph node circulation. These monocytes, upon moving to injured tissue, turn into dendritic cells and macrophages while promoting tissue healing. The spleen is a center of activity of the mononuclear phagocyte system and is sometimes compared to a large lymph node, as its absence causes a predisposition to certain infections. Up to a quarter of the body's lymphocytes are stored in the spleen at any given time.

The “lymph nodes” (“lymph glands”) have the highest lymphocyte concentration, however. A “lymph node” is a kidney-shaped organ of the lymphatic system and the adaptive immune system. A large number of lymph nodes are linked throughout the body by the lymphatic vessels. They are major sites of lymphocytes that include B and T cells. Lymph nodes are essential for the proper functioning of the immune system, acting as filters for foreign particles, including cancer cells, but have no detoxification function. A lymph node is enclosed in a fibrous capsule and comprises an outer cortex and an inner medulla. Lymph nodes become inflamed or enlarged in various diseases, from minor infections to cancers. The condition of lymph nodes is critical in cancer staging, which decides the treatment to be used and determines the prognosis, as the presence of cancer cells in lymph nodes near the primary cancer tumor indicates metastasis. After entering the lymph node from afferent lymphatic vessels, lymph flows into a space underneath the subcapsular sinus capsule, then into cortical sinuses. After passing through the cortex, lymph then collects in medullary sinuses and drains into the efferent lymph vessels to exit the node at the hilum on the concave side. There are about 450 lymph nodes in the adult human. A lymph node is divided into nodules (or lobules), each consisting of a region of cortex with combined follicle B cells, a paracortex of T cells, and a part of the nodule in the medulla. The substance of a lymph node is divided into the outer cortex and the inner medulla. The cortex of a lymph node is the outer portion of the node, underneath the capsule and the subcapsular sinus. It has an outer part and a deeper part known as the paracortex. The outer cortex consists of groups of mainly inactivated B cells called follicles. When activated, these may develop into what is called a germinal center. The deeper paracortex primarily consists of the T cells, which interact with dendritic cells, and the dense reticular network. The medullary cords are cords of lymphatic tissue and include plasma cells, macrophages, and B cells. Lymph nodes contain lymphocytes, a type of white blood cell, and are primarily B cells and T cells. B cells are mainly found in the outer cortex, clustered together as follicular B cells in lymphoid follicles, and T cells and dendritic cells are found primarily in the paracortex. The reticular network provides structural support and a surface for the adhesion of dendritic cells, macrophages, and lymphocytes. It also allows the exchange of material with blood through the high endothelial venules and provides the growth and regulatory factors necessary for activation and maturation of immune cells.

The primary function of lymph nodes is the filtering of lymph to identify and fight infection. To accomplish this, lymph nodes contain lymphocytes, including B cells and T cells, which circulate through the bloodstream and enter and reside in lymph nodes. B cells produce antibodies. Each antibody has a single predetermined target, an antigen, to which it can bind. These circulate throughout the bloodstream, and if they find this target, the antibodies bind to it and stimulate an immune response. Each B cell produces different antibodies. B cells enter the bloodstream as “naïve” cells produced in the bone marrow. After entering a lymph node, they enter a lymphoid follicle, where they divide, each with a different antibody. If a cell is stimulated, it will produce more antibodies (a plasma cell) or act as a memory cell to help the body fight future infection. If a cell is not stimulated, it will undergo apoptosis and die.

B cells acquire antigen directly from the afferent lymph. If a B cell binds its cognate antigen, it will be activated. Some B cells will immediately develop into antibody-secreting plasma cells and secrete IgM. Other B cells will internalize the antigen and present it to follicular helper T cells (follicular B helper T cells; TFH; T_{FH}) on the B and T cell zone interface. If a cognate TFH is found, it will upregulate CD40L and promote somatic hypermutation and isotype class switching of the B cell, increasing its antigen-binding affinity and changing its effector function. The proliferation of cells within a lymph node will make the node expand. “Lymphadenopathy” refers to glands that are enlarged or swollen.

Antigens are molecules found on bacterial cell walls or on viruses, chemical substances secreted from bacteria, or sometimes even molecules present in body tissue itself. These are taken up by cells throughout the body called antigen-presenting cells, such as dendritic cells. These antigen-presenting cells enter the lymph system and then lymph nodes. They present the antigen to T cells and, if there is a T cell with the appropriate T cell receptor, it will be activated.

The “tonsils” are a set of secondary lymphoid organs facing into the aerodigestive tract, which is known as Waldeyer's tonsillar ring and consists of the adenoid tonsil, two tubal tonsils, two palatine tonsils, and the lingual tonsils. These organs play an essential role in the immune system. On their surface, the tonsils have specialized antigen-capture cells called “Microfold cells” (M cells) that allow for the uptake of antigens produced by pathogens. These M cells then alert the B cells and T cells in the tonsil that a pathogen is present, and an immune response is stimulated. As a result, b cells are activated and proliferate in the germinal centers, where B memory cells are created, and secretory antibody (IgA) is produced of the tonsils. The tonsils also produce T cells like the thymus.

The “appendix” is a finger-like, blind-ended tube connected to the cecum, a pouch-like structure of the colon, located at the junction of the small and the large intestines. The appendix aids in the proper movement and removal of waste matter in the digestive system, serves as a reservoir of beneficial gut microbes and contains lymphatic vessels that regulate pathogens. The appendix, which comprises lymphoid tissue, has been identified as an essential component of mammalian mucosal immune function, particularly B-cell-mediated immune responses and extrathymically derived T-cells. In addition, innate lymphoid cells function in the gut to help the appendix maintain digestive health.

“Tertiary lymphoid organs” (TLOs) (“tertiary lymphoid structures” [TLS]; “ectopic lymphoid structures” [ELS]) are abnormal lymph node-like structures that form in peripheral tissues at sites of chronic inflammation, such as chronic infection, transplanted organs undergoing graft rejection, some cancers, and autoimmune and autoimmune-related diseases. TLOs are regulated differently from the normal process whereby lymphoid tissues are formed during ontogeny, dependent on cytokines and hematopoietic cells, but still, drain interstitial fluid and transport lymphocytes in response to the same chemical messengers and gradients. TLOs typically contain far fewer lymphocytes and assume an immune role only when challenged with antigens that result in inflammation. They achieve this by importing the lymphocytes from blood and lymph. TLOs often have an active germinal center, surrounded by a network of follicular dendritic cells (FDCs). TLOs play a vital role in the immune response to cancer. While some patients with TLOs in the vicinity of their tumors have a better prognosis, the opposite is true for other cancers. The immune response against the tumor, which the TLOs mediate, may improve the outcome in some cancer patients. TLOs with an active germinal center tend to have a better prognosis than those with TLOs without a germinal center. TLOs may also promote an anti-tumor response when patients are treated with immunotherapy.

Lymphoid tissue associated with the lymphatic system is concerned with immune functions in defending the body against infections and the spread of tumors, and it comprises connective tissue formed of reticular fibers, with various types of leukocytes (white blood cells), mostly lymphocytes enmeshed in it, through which the lymph passes. Regions of the lymphoid tissue that are densely packed with lymphocytes are known as “lymphoid follicles.” In addition, lymphoid tissue can either be structurally well organized as lymph nodes or consist of loosely organized lymphoid follicles known as the “mucosa-associated lymphoid tissue” (MALT).

The central nervous system also has lymphatic vessels. Functional “meningeal lymphatic vessels” lining the dural sinuses, anatomically integrated into the membrane surrounding the brain and providing T-cell gateways into and out of the meninges.

“Lymphatic vessels” or “lymph vessels” are thin-walled vessels that conduct lymph between different body parts. They include the tubular vessels of the lymph capillaries and the larger collecting vessels—the right lymphatic duct and the thoracic duct (the left lymphatic duct). Lymph vessels that carry lymph to a lymph node are “afferent lymph vessels,” and those that carry it from a lymph node are “efferent lymph vessels,” from where the lymph may travel to another lymph node, may be returned to a vein, or may travel to a larger lymph duct. Lymph ducts drain the lymph into one of the subclavian veins and thus return it to general circulation. The lymph capillaries are mainly responsible for absorbing interstitial fluid from the tissues.

In contrast, lymph vessels propel the absorbed fluid forward into the larger collecting ducts, ultimately returning to the bloodstream via one of the subclavian veins. Lymphatic circulation begins with a blind-ending (closed at one end) highly permeable superficial “lymph capillaries,” formed by endothelial cells with button-like junctions between them that allow fluid to pass through them when the interstitial pressure is sufficiently high. Lymph capillaries have many interconnections (anastomoses) between them and form an outstanding network. The lymph system collaborates with white blood cells in lymph nodes to protect the body from being infected by cancer cells, fungi, viruses, or bacteria. This is known as a secondary circulatory system.

“Lymph” is the fluid that flows through the lymphatic system. “Interstitial fluid,” comprising the fluid between the cells in all body tissues, enters the lymph capillaries. This lymphatic fluid is then transported via progressively larger lymphatic vessels through lymph nodes, where tissue lymphocytes remove substances and circulating lymphocytes are added to the fluid, before emptying ultimately into the right or the left subclavian vein mixes with central venous blood. As a result, the composition of lymph continually changes. Lymph returns proteins and excess interstitial fluid to the bloodstream. It also transports fats from the digestive system to the blood via chylomicrons. Bacteria may enter the lymph channels and be transported to lymph nodes, where the bacteria are destroyed. Metastatic cancer cells can also be transported via lymph.

“High endothelial venules” (HEV) are specialized post-capillary venous swellings characterized by plump endothelial cells as opposed to the usual thinner endothelial cells found in regular venules. HEVs enable lymphocytes circulating in the blood to directly enter a lymph node (by crossing through the HEV). In humans, HEVs are found in all secondary lymphoid organs (except for the spleen), including hundreds of lymph nodes dispersed in the body, tonsils, and adenoids in the pharynx, Peyer's patches (PIs) in the small intestine, appendix, and small aggregates of lymphoid tissue in the stomach and large intestine. Stromal cells are precursors of HEV. Unlike endothelial cells from other vessels, the high endothelial cells of HEVs have a distinctive appearance, consisting of a cuboidal morphology and various receptors to interact with leukocytes (e.g., express specialized ligands for lymphocytes and support high levels of lymphocyte extravasation). HEVs enable naïve lymphocytes to move in and out of the lymph nodes from the circulatory system. HEV cells express addressins, which are specific adhesion molecules that attach to the L-selectins on lymphocytes and anchor them to the HEV wall in preparation for crossing the endothelium. When an antigen-presenting cell (APC), such as a dendritic cell, binds a foreign antigen, it becomes activated. It moves into the lymph nodes (sites for antigen sampling by T cells) via afferent lymphatic vessels. Naïve T cells in the circulation regularly move through the lymph nodes via HEV to scan the APC for foreign antigens. When they encounter such an antigen, the cell becomes activated, resulting in the immune system mounting a response against the causative agent of the infection.

Disclosed herein are methods for forming biomaterial scaffolds comprising hydrogel compositions comprising cells and one or more other therapeutic and/or biotherapeutic agents (e.g., chemotherapies, monoclonal antibody [mAb, moAb] therapies, chimeric antigen receptor T-cells [CAR-T], apoptotic therapies [e.g., with apoptotic cells, supernatants, proteins], small interfering ribonucleic acid [siRNA] therapies, micro ribonucleic acid [miRNA] treatments, antisense therapies, metabolic therapies, and/or inhibitor therapies). In some embodiments, the biomaterial scaffold is in vitro, in situ, or in vivo. In some embodiments, the method comprises formation of a lymphatic tissue. In some embodiments, the method consists of the formation of a high endothelial venule (HEV).

### Cells

In some embodiments, the cell is non-immunogenic. In some embodiments, the cell is a universal donor cell. In some embodiments, the cell is a non-immunogenic universal donor cell.

In some embodiments, the hydrogel comprises a platform for maintenance, growth, or proliferation of a cell or a medium for maintenance, growth, spread, or proliferation. In some embodiments, the hydrogel uses the maintenance, growth, spread, or proliferation of cells formulated on or in, or encapsulated within the hydrogel. In some embodiments, the maintenance, development, spread, or proliferation of cells comprises growth or repair of a lymphatic vessel or lymph node; a blood vessel; a tissue; or an organ or a part of an organ. In some embodiments, the maintenance, growth, spread, or proliferation of cells comprises growth repair of a lymphatic vessel or lymph node; a blood vessel; a tissue; or an organ or a part of an organ. In some embodiments, the hydrogel composition comprises a payload for treatment.

“Stromal cells” (“mesenchymal stromal cells”) are differentiating cells that can become connective tissue cells of any organ but are commonly found in the bone marrow and other parts of the lymphatic system. Stromal cells, which have diverse functions, are an important part of the body's immune response and modulate inflammation through multiple pathways. They also aid in the differentiation of hematopoietic cells and forming necessary blood elements. Additionally, stromal cells play a role in inflammation responses and control the number of cells accumulating at an inflamed tissue region. Stromal cells originate from multipotent mesenchymal stem cells.

“Lymph node stromal cells” are essential to the structure and function of the lymph node. Their functions include, but are not limited to, creating an internal tissue scaffold for the support of hematopoietic cells; the release of small molecule chemical messengers that facilitate interactions between hematopoietic cells; the facilitation of the migration of hematopoietic cells; the presentation of antigens to immune cells at the initiation of the adaptive immune system; and the homeostasis of lymphocyte numbers. Types of lymph node stromal cells include fibroblastic reticular cells (FRC), follicular dendritic cells (FDC), marginal reticular cells (MRC), lymphatic endothelial cells (LEC), high endothelial cells (HEC), and alpha-7 integrin pericytes (AIP).

“Fibroblastic reticular cells” (“fibroblast reticular cells”; FRC) are a type of lymph node stromal cell located in the T-cell zone of the cortex. FRCs produce collagen alpha-1 (III) rich reticular fibers that form a dense network within the lymphoid tissue. These are connected by collagen XIV, small leucine-rich proteoglycans, and lysyl oxidase. The network of fibers supports and guides the movement of dendritic cells (DCs), T-cells, and B-cells and creates a porous molecular sieve in the lymph node. FRCs express chemokines that assist the activity of some T-cells and dendritic cells.

“Adipose cells” (“adipocytes”; “lipocytes”; “fat cells”) are the cells that primarily compose adipose tissue, specialized in storing energy as fat. Like stromal cells, adipocytes are derived from mesenchymal stem cells which give rise to adipocytes through adipogenesis. There are two types of adipose tissue, white adipose tissue (WAT) and brown adipose tissue (BAT), which are also known as white and brown fat, respectively, and comprise two types of fat cells. A third type of fat cells is the marrow adipocyte.

“Stem cells” are undifferentiated or partially differentiated cells that can differentiate into various types of cells and proliferate indefinitely to produce more of the same stem cell. They are the earliest type of cell in a cell lineage. They are found in both embryonic and adult organisms, but have slightly different properties in each. They are usually distinguished from progenitor cells, which cannot divide indefinitely, and precursor or blast cells, which are usually committed to differentiating into one cell type. Stem cell characteristics include self-renewal and multipotency (multidifferentiative potential). “Adult stem cells” are found in a few specific locations in the body, known as niches, such as those in the bone marrow or gonads. They replenish rapidly lost cell types and are multipotent (differentiate into a number of cell types, but only those of a closely related family of cells), oligopotent (only differentiate into a few cell types, such as lymphoid or myeloid stem cells), or unipotent (only differentiate into one cell type while still retaining self-renewal properties). In mammals, they include, but are not limited to, hematopoietic stem cells (which replenish blood and immune cells), basal cells (which maintain the skin epithelium), and mesenchymal stem cells (which maintain bone, cartilage, muscle, and fat cells). Mesenchymal stem cells (MSC) are multipotent and are found, e.g., in the muscle, liver, bone marrow. MSC can differentiate into numerous cell categories, including, but not limited to, adipocytes, osteocytes, and chondrocytes, derived by the mesodermal layer.

In some embodiments, the hydrogel composition comprises a cell. In some embodiments, the cell comprises a stromal cell, an adipose cell, a natural killer cell (NKC), a mesenchymal stem cell (MSC), or a stem cell. In some embodiments, the stromal cell comprises a fibroblastic reticular cell (FRC), a follicular dendritic cell (FDC), a marginal reticular cell (MRC), a lymphatic endothelial cell (LEC), a high endothelial cell (HEC), or an alpha-7 integrin pericyte (AIP).

“Lymphocytes” are a type of white blood cell in the immune system of jawed vertebrates. Lymphocytes include natural killer (NK) cells (which function in cell-mediated, cytotoxic innate immunity), T-cells (for cell-mediated, cytotoxic adaptive immunity), and B-cells (for humoral, antibody-driven adaptive immunity), which are derived from a common progenitor. They are the main type of cell found in lymph and have a large nucleus. Lymphocytes comprise approximately 18%-42% of circulating white blood cells (“leukocytes”).

“T-cells” (“T cells”; “T lymphocytes”) are lymphocytes that play a central role in the adaptive immune response. T-cells can be distinguished from other lymphocytes by the presence of a “T-cell receptor” (TCR) on their cell surface. Subtypes of T-cells include, but are not limited to, helper CD4+ T-cells, cytotoxic CD8+ T-cells, memory T-cells, regulatory CD4+ T-cells, natural killer T-cells (not the same as natural killer cells), mucosal associated invariant T-cells, and gamma delta T-cells. A key function of T-cells is immune-mediated cell death, and it is carried out by two major subtypes: CD8+ “killer” and CD4+ “helper” T-cells. CD8+ T-cells, also known as “killer T-cells”, are cytotoxic, because they are able to directly kill virus-infected cells, as well as cancer cells. CD8+ T-cells are also able to use “cytokines” (small signaling proteins) to recruit other types of cells when mounting an immune response. CD4+ T-cells, function as “helper cells”. Unlike CD8+ killer T cells, CD4+ helper T cells function by indirectly killing cells identified as foreign: they determine if and how other parts of the immune system respond to a specific, perceived threat. Helper T cells also use cytokine signaling to influence regulatory B cells directly, and other cell populations indirectly. “Memory T-cells” are a long-lived antigen-specific subset of T-cells.

The “regulatory T-cells” (“T regulatory cells”; “regulatory CD4+ T-cells”; Treg cells; Treg), formerly known as “suppressor T-cells,” are a subpopulation of T-cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FOXP3, and CD25.

“B-cells” (“B cells”; “B lymphocytes”) are lymphocytes that function in the humoral immunity component of the adaptive immune system. B-cells produce antibody molecules, but instead of being secreted, the antibody molecules are inserted into the plasma membrane where they serve as a part of B-cell receptors. When a naïve or memory B-cell is activated by an antigen, it proliferates and differentiates into an antibody-secreting effector cell, known as a plasmablast or plasma cell. Additionally, B-cells present antigens (also classified as professional “antigen-presenting cells” (APCs)) and secrete cytokines. B-cells express “B-cell receptors” (BCRs) on their cell membrane. BCRs allow the B-cell to bind to a specific antigen, against which it will initiate an antibody response. Types of B-cells include, but are not limited to, plasmablasts, plasma cells, lymphoplasmacytoid cells, memory B-cells, B-2 cells (subtypes: follicular B-cells and marginal zone B-cells), B-1 cells, and regulatory B-cells. “Memory B-cells” (MBC) are long-lived B-cells that develop within germinal centers of the secondary lymphoid organs. Memory B-cells circulate in the blood stream in a quiescent state, sometimes for decades. If the memory B-cell later encounters the same antigen during a subsequent infection, it triggers an accelerated and robust secondary immune response. Memory B-cells have “B cell receptors” (BCRs) on their cell membrane, identical to the one on their parent cell, that allow them to recognize antigen and mount a specific antibody response.

“Regulatory B-cells” (“B regulatory cells”; Breg cells; Breg) represent a small population of B-cells which participates in immunomodulations and in suppression of immune responses by various mechanisms. The main mechanism is a production of anti-inflammatory cytokine interleukin 10 (IL-10).

“Natural killer cells” (“large granular lymphocytes” [LGL]; NK cells; NK), are a type of cytotoxic lymphocyte critical to the innate immune system that belong to the family of innate lymphoid cells (ILC). NK cells provide rapid responses to virus-infected cell and other intracellular pathogens acting at around 3 days after infection and respond to tumor formation. Typically, immune cells detect the “major histocompatibility complex” (MHC) presented on infected cell surfaces, triggering cytokine release, causing the death of the infected cell by lysis or apoptosis. NK cells are unique, however, as they have the ability to recognize and kill stressed cells in the absence of antibodies and MHC, allowing for a much faster immune reaction. NK cells can be classified as CD56bright or CD56dim. CD56bright NK cells release cytokines and constitute the majority of NK cells, being found in bone marrow, secondary lymphoid tissue, liver, and skin. CD56dim NK cells are primarily found in the peripheral blood and are characterized by their cell killing ability. CD56dim NK cells are always CD16 positive (CD16 is the key mediator of antibody-dependent cellular cytotoxicity [ADCC]), and CD56bright can transition into CD56dim by acquiring CD16. NK cells can eliminate virus-infected cells via CD16-mediated ADCC.

### Cytokines, Chemokines, and Other Proteins

In some embodiments, the hydrogel composition comprises a therapeutic or biotherapeutic. In some embodiments, the hydrogel composition comprises a pharmaceutical composition, a cytokine or a chemokine, a modulating agent, a targeting agent, or a stabilizing agent.

In some embodiments, the modulating agent comprises an inhibitor or an activator. In some embodiments, the modulating agent comprises an immunomodulating agent.

A “modulating agent” is a substance that stimulates or suppresses physiological responses and may help in the treatment of a disease or abnormal physiological condition. For example, a cytokine or chemokine modulating agent may bind to a receptor (e.g., on the surface of a cell) to stimulate or suppress a physiological response (e.g., by activation or inhibition) An antibody or antigen-binding domain modulating agent binds to an antigen, thereby stimulating or suppressing a physiological response (e.g., stimulating an immune response). A fragment of DNA or RNA can be selected to bind to a complementary fragment, which may inhibit expression of the encoded protein.

An “immunomodulating agent” (“immune system modulator”) is a substance that stimulates or suppresses the immune system and may help the body fight cancer, infection, or other diseases. Specific immunomodulating agents, such as monoclonal antibodies, cytokines, and vaccines, affect specific parts of the immune system. Nonspecific immunomodulating agents, such as BCG and levamisole, affect the immune system in a general way.

An “immune checkpoint inhibitor” (“checkpoint inhibitor”) is a type of pharmaceutical or biopharmaceutical agent that blocks checkpoint proteins that are made by some types of immune system cells, such as T cells, and by some cancer cells. These checkpoints inhibit immune responses from being too strong and sometimes can inhibit T cells from killing cancer cells. When these checkpoints are blocked, T cells can kill cancer cells more efficiently. Examples of checkpoint proteins found on T cells or cancer cells include, but are not limited to, PD-1/PD-L1 and CTLA-4/B7-1/B7-2.

Cytotoxic T-lymphocyte-associated protein 4 (CTLA4 or CTLA-4; CD152 [cluster of differentiation 152]) is a protein receptor that functions as an immune checkpoint and downregulates immune responses. CTLA-4 is constitutively expressed in regulatory T cells, but only upregulated in conventional T cells after activation. This situation is particularly observed in some cancers. For example, it can serve as an “off” switch when bound to CD80 or CD86 on the surface of antigen-presenting cells. Inhibitors of CTLA-4 include, but are not limited to, ipilimumab.

In some embodiments, the hydrogel composition comprises an anti-CTLA-4 antibody or an anti-CTLA-4-antigen-binding domain. In some embodiments, the hydrogel composition comprises a biopharmaceutical composition comprising ipilimumab.

Programmed cell death protein 1 (PD-1; cluster of differentiation 279 [CD279]), a cell surface protein, a member of the immunoglobulin superfamily, is expressed on T cells and pro-B cells and promotes self-tolerance by suppressing T-cell inflammatory activity, preventing autoimmune diseases, but also inhibiting the immune system from killing cancer cells. Programmed cell death protein 1 (PD-1; cluster of differentiation 279 [CD279]), is a cell surface protein that has a role in regulating the immune system's response to the cells of the human body by down-regulating the immune system and promoting self-tolerance by suppressing T cell inflammatory activity. An immune checkpoint protein, PD-1 promotes apoptosis of antigen-specific T-cells in lymph nodes and reduces apoptosis in regulatory T-cells (Tregs). PD-1 is a cell surface receptor that belongs to the immunoglobulin superfamily and is expressed on T cells and pro-B cells. PD-1 binds two ligands, programmed death-ligand 1 (PD-L1) and programmed death-ligand 2 (PD-L2). Inhibitors of PD-1 include, but are not limited to, nivolumab, pembrolizumab, and cemiplimab.

In some embodiments, the hydrogel composition comprises an anti-PD-1 antibody or an anti-PD-1-antigen-binding domain. In some embodiments, the hydrogel composition comprises a biopharmaceutical composition comprising nivolumab, pembrolizumab, or cemiplimab.

Programmed death-ligand 1 (PD-L1) is highly expressed on the surface of cells of some types of cancers, including, but not limited to, melanoma, bladder cancer, and gastric cancer. As a result, PD-1 inhibitors block PD-1 and lower immune system activation when attacking tumors. Inhibitors of PD-L1 include, but are not limited to, atezolizumab, avelumab, and durvalumab.

In some embodiments, the hydrogel composition comprises an anti-PD-L1 antibody or an anti-PD-L1 antigen-binding domain. In some embodiments, the hydrogel composition comprises a biopharmaceutical composition comprising atezolizumab, avelumab, or durvalumab.

In certain embodiments, the hydrogel composition comprises an anti-PD-1 antibody or an anti-PD-1 antigen-binding domain in combination with either (a) an anti-PD-L1 antibody or an anti-PD-L1 antigen-binding domain, (b) an anti-CTLA-4 antibody or an anti-CTLA-4 antigen-binding domain, or (c) an anti-RANKL antibody or an anti-RANKL antigen-binding domain.

“Receptor activator of nuclear factor kappa-B ligand” (“RANKL”) (“tumor necrosis factor ligand superfamily member 11” [“TNFSF11”]; “TNF-related activation-induced cytokine” [“TRANCE”]; “osteoprotegerin ligand” [“OPGL”]; “osteoclast differentiation factor” [“ODF”]) is a type II membrane protein and is a member of the tumor necrosis factor (TNF) cytokine superfamily. RANKL is found on the surface of stromal cells, osteoblasts, and T cells. It affects the immune system and control bone regeneration and remodeling. RANKL is an apoptosis regulator gene, a binding partner of osteoprotegerin (OPG), a ligand for the receptor RANK and controls cell proliferation by modifying protein levels of Id4, Id2 and cyclin D1. RANKL also has a function in the immune system, where it is expressed by T helper cells and is thought to be involved in dendritic cell maturation. It is a dendritic cell survival factor and helps regulate T cell-dependent immune responses. T cell activation induces RANKL expression and can lead to an increase of osteoclastogenesis and bone loss. RANKL can also activate the antiapoptotic kinase AKT/PKB through a signaling complex involving SRC kinase and tumor necrosis factor receptor-associated factor 6 (TRAF6), indicating that RANKL may have a role in the regulation of apoptosis. A further role for RANKL in immunity was found in sinusoidal macrophages in lymph nodes that alert the immune system to lymph-borne antigens. In some cancer patients, in addition to directly signaling through RANK for macrophage differentiation, RANKL activates the adjacent lymphatic endothelial cells to create a niche environment for these specialized immune cells. RANKL surface expression and secreted RANKL expression was reported to be increased, 80% and 50% respectively. Therefore, RANKL is considered to be a key signal regulator for cancer-induced bone loss. Overproduction of RANKL is also implicated in a variety of degenerative bone diseases, such as rheumatoid arthritis and psoriatic arthritis. The osteoclast cell surface “receptor activator of nuclear factor kappa-B” (“RANK”) binds to RANKL, and the osteocyte is the major source of RANKL regulating bone remodeling.

“Osteoprotegerin” (“OPG”) (“osteoclastogenesis inhibitory factor” [“OCIF”]; “tumor necrosis factor receptor superfamily member 11B” [“TNFRSF11B”]), is a cytokine receptor of the tumor necrosis factor (TNF) receptor superfamily. OPG has been identified as having a role in tumor growth and metastasis, heart disease, immune system development and signaling, mental health, diabetes, and the prevention of pre-eclampsia and osteoporosis during pregnancy. It serves as a decoy receptor for RANKL. Upregulation of OPG has been implicated in cancer metastasis and angiogenesis, as well as in multiple myeloma.

According to the vicious cycle hypothesis, after secondary tumors cells have migrated to bone, the tumor cell will secrete cytokines and growth factors that can act on osteoblast lineage cells. Since osteoblasts control the regulation of RANKL, the stimulation via cytokines and growth factors will then stimulate osteoblasts to increase the expression of RANKL, often while simultaneously reducing bone formation. The additional RANKL-mediated osteoclast frequency and activity will in turn increase secretion of growth factors, or matrix derived factors, which can ultimately increase tumor growth and bone destruction activity.

In some embodiments, the hydrogel composition comprises an anti-RANKL agent. In some embodiments, the anti-RANKL agent comprises an anti-RANKL antibody or antigen-binding domain. In some embodiments, the anti-RANKL agent comprises denosumab or a bisphosphonate.

A “targeting agent” is a substance that binds to a specific target molecule. For example, a cytokine or chemokine targeting agent may bind to a receptor (e.g., on the surface of a cell), and conversely, a receptor targeting agent may bind to a cytokine or chemokine. An antibody or antigen-binding domain targeting agent binds to an antigen, and conversely, an antigen targeting agent binds to an antibody or antigen-binding domain. A fragment of DNA or RNA can be selected to bind to a complementary fragment.

A targeting agent can be linked to, or otherwise combined with, a modulating agent in order to deliver the modulating agent to the appropriate location (e.g., a targeting agent that targets a receptor on a cancer cell can be linked with a modulating agent that inhibits the growth, division, or metastasis of the cancer cell or causes the cancer cell to undergo programmed cell death.

A “stabilizing agent” (“stabilizer,” “stabilizing excipient”) is a substance used to help the active pharmaceutical ingredient (API) maintain the desirable properties of the product until it is administered to the subject.

In some embodiments, the hydrogel composition comprises a cytokine. In some embodiments, the hydrogel composition comprises a chemokine.

In some embodiments, immune cells, for example T-cells, are generated and expanded by the presence of cytokines in the hydrogel composition. In some embodiments, cytokines that affect generation and maintenance to T-helper cells in vivo comprise IL-2, IL-12, and IL-15. In some embodiments, T regulatory (Treg) cells are generated from naïve T cells by cytokine induction in vivo. In some embodiments, transforming growth factor-beta (TGF-beta, TGF-β) and/or IL-2 play a role in differentiating naïve T cell to become Treg cells.

“Cytokines” are a category of small proteins (˜5-20 kDa) critical to cell signaling. Cytokines are peptides and usually are unable to cross the lipid bilayer of cells to enter the cytoplasm. Among other functions, cytokines may be involved in autocrine, paracrine and endocrine signaling as immunomodulating agents. Cytokines may be pro-inflammatory or anti-inflammatory. Cytokines include, but are not limited to, chemokines (cytokines with chemotactic activities), interferons, interleukins (ILs; cytokines made by one leukocyte and acting on one or more other leukocytes), lymphokines (produced by lymphocytes), monokines (produced by monocytes), and tumor necrosis factors. Cells producing cytokines include, but are not limited to, immune cells (e.g., macrophages, B lymphocytes, T lymphocytes and mast cells), as well as endothelial cells, fibroblasts, and various stromal cells. A particular cytokine may be produced by more than one cell type. Cytokines have been shown to be involved in autocrine, paracrine and endocrine signaling as immunomodulating agents. They act through cell surface receptors and are especially important in the immune system, modulating the balance between humoral and cell-based immune responses. Adverse effects of cytokines have been implicated in Alzheimer's disease.

A skilled artisan would appreciate that the term “cytokine” may encompass cytokines beneficial to enhancing an immune response targeted against a cancer or a pre-cancerous or non-cancerous tumor or lesion. A skilled artisan would also appreciate that the term “cytokine” may encompass cytokines beneficial to enhancing an immune response against a disease or inflammation (e.g., resulting from surgery, an injury, or damage from an autoimmune response) or that the term “cytokine” may encompass cytokines beneficial to reducing an abnormal autoimmune response.

In some embodiments, a cytokine encoded by the nucleic acid expands and maintains T-helper cells (helper T cells). In some embodiments, a cytokine encoded by the nucleic acid expands T-helper cells. In some embodiments, a cytokine encoded by the nucleic acid maintains T-helper cells. In some embodiments, a cytokine encoded by the nucleic acid expands cytotoxic T cells (CTLs). In some embodiments, a cytokine encoded by the nucleic acid activates cytotoxic T cells. In some embodiments, a cytokine encoded by the nucleic acid expands and activates cytotoxic T cells. In some embodiments, a cytokine encoded by the nucleic acid increases proliferation of a T-helper cell population. In some embodiments, a cytokine encoded by the nucleic acid increases proliferation of a cytotoxic T cell population.

“Inflammatory cytokines” (or proinflammatory cytokines), as used herein, are a type of signaling molecule (a cytokine) that is secreted from immune cells like helper T cells (Th) and macrophages, and certain other cell types that promote inflammation. They include interleukin-1 (IL-1), IL-12, and IL-18, tumor necrosis factor alpha (TNF-α), interferon gamma (IFNγ), and granulocyte-macrophage colony stimulating factor (GM-CSF) and play an important role in mediating the innate immune response. Inflammatory cytokines are predominantly produced by and involved in the upregulation of inflammatory reactions.

“Anti-inflammatory cytokines,” as used herein, are cytokines that counterbalance the effects of inflammatory cytokines. Major anti-inflammatory cytokines include interleukin (IL)-1 receptor antagonist, IL-4, IL-6, IL-10, IL-11, and IL-13. Specific cytokine receptors for IL-1, tumor necrosis factor-alpha, and IL-18 can also function as proinflammatory cytokine inhibitors.

In some embodiments, the cytokine comprises an interleukin (IL). A skilled artisan would appreciate that interleukins comprise a large family of molecules, including, but not limited to, IL-1, IL-2, IL-3, IL-4, IL-5, IL-6, IL-7, IL-8, IL-9, IL-10, IL-11, IL-12, IL-13, IL-14, IL-15, IL-16, IL-17A, IL-17F, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36.

In some embodiments, the encoded interleukin comprises an IL-2, IL-12, or an IL-15, or any combination thereof. In some embodiments, the encoded cytokine comprises an IL-2. In some embodiments, the encoded cytokine comprises an IL-12. In some embodiments, the encoded cytokine comprises an IL-15.

“Cytokine release syndrome” (CRS), as used herein, is a form of systemic inflammatory response syndrome (SIRS) that can be triggered by a variety of factors such as infections and certain drugs. It refers to cytokine storm syndromes (CSS) and occurs when large numbers of white blood cells are activated and release inflammatory cytokines, which in turn activate yet more white blood cells. CRS is also an adverse effect of some monoclonal antibody medications, as well as adoptive T-cell therapies. When occurring as a result of a medication, it is also known as an infusion reaction. The term “cytokine storm” is often used interchangeably with CRS but, although they have similar clinical phenotypes, their characteristics are different. When occurring as a result of a therapy, CRS symptoms may be delayed until days or weeks after treatment. Immediate-onset CRS is a cytokine storm.

“Chemokines” are a family of small cytokines, or signaling proteins secreted by cells. They have the ability to induce directed chemotaxis in nearby responsive cells; they are chemotactic cytokines. Chemokines regulate cell migration, such as attracting immune cells to a site of infection or injury. Some chemokines are considered pro-inflammatory and can be induced during an immune response to recruit cells of the immune system to a site of infection, while others are considered homeostatic and are involved in controlling the migration of cells during normal processes of tissue maintenance or development. Chemokines are found in all vertebrates. Chemokines have been classified into four main subfamilies: CXC, CC, CX3C and C. All of these proteins exert their biological effects by interacting with G protein-linked transmembrane receptors called chemokine receptors, that are selectively found on the surfaces of their target cells. Their release is often stimulated by pro-inflammatory cytokines such as interleukin 1. Inflammatory chemokines function mainly as chemoattractants for leukocytes, recruiting monocytes, neutrophils and other effector cells from the blood to sites of infection or tissue damage. Certain inflammatory chemokines activate cells to initiate an immune response or promote wound healing. They are released by many different cell types and serve to guide cells of both innate immune system and adaptive immune system. “Homeostatic chemokines” are constitutively produced in certain tissues and are responsible for basal leukocyte migration, while “inflammatory chemokines” are formed under pathological conditions (on pro-inflammatory stimuli, such as IL-1, TNF-alpha, LPS, or viruses) and actively participate in the inflammatory response attracting immune cells to the site of inflammation, but some chemokines fall into both categories. Homeostatic chemokines are basal produced in the thymus and lymphoid tissues. Chemokines of various types attract monocytes/macrophages, T-cells, mast cells, eosinophils, and neutrophils.

“Lymphotoxin-alpha” (LT-alpha, LT-α) (tumor necrosis factor-beta (TNF-β)), a member of the tumor necrosis factor superfamily, is a cytokine produced by lymphocytes. Belonging to the hematopoietic cell line, LT-alpha exhibits anti-proliferative activity and causes the cellular destruction of tumor cell lines. As a cytotoxic protein, LT-alpha performs a variety of important roles in immune regulation depending on the form that it is secreted as. Unlike other members of the TNF superfamily, LT-alpha is only found as a soluble homotrimer, when found at the cell surface it is found only as a heterotrimer with lymphotoxin-beta (LT-beta; LTβ). LT-alpha has a significant impact on the maintenance of the immune system including the development of secondary lymphoid organs. LT-alpha mediates a large variety of inflammatory, immunostimulatory, and antiviral responses. As a signaling molecule, LT-alpha is involved in the regulation of cell survival, proliferation, differentiation, and apoptosis. LT-alpha plays an important role in innate immune regulation and its presence has been shown to prevent tumor growth and destroy cancerous cell lines. In contrast, unregulated expression of LT-alpha can result in a constantly active signaling pathway, thus leading to uncontrolled cellular growth and creation of tumors. Furthermore, LT-alpha effects depend on the type of organ it acts upon, type of cancer cells, cellular environment, gender, and time of effect during an immune response. “Lymphotoxin-beta” (LT-beta, LT-β) is a type II membrane protein of the tumor necrosis factor family. It anchors LT-alpha to the cell surface through heterotrimer formation. The predominant form on the lymphocyte surface is the lymphotoxin-alpha 1/beta 2 complex (LT-alpha1-beta2, LT-α1β2, LT-α₁β₂) (e.g., 1 molecule alpha/2 molecules beta), and this complex is the primary ligand for the LT-beta receptor. The minor complex is LT-alpha 2/beta 1 (LT-alpha2-beta1, LT-α2β1, LT-α₂β₁). LT-beta is an inducer of the inflammatory response system and involved in normal development of lymphoid tissue. LT-alpha1-beta2 can interact with receptors such as LT-beta receptors.

In some embodiments, the hydrogel composition comprises LT-alpha, LT-beta, LT-alpha1-beta2, or LT-alpha2-beta1.

“Chemokine (C-C motif) ligand 20” (“CCL20”) (“liver activation regulated chemokine” [“LARC”]; “macrophage inflammatory protein-3” [“MIP3A”]) is a small cytokine belonging to the CC chemokine family. It is strongly chemotactic for lymphocytes and weakly attracts neutrophils. CCL20 is expressed in several tissues with highest expression observed in peripheral blood lymphocytes, lymph nodes, liver, appendix, and fetal lung and lower levels in thymus, testis, prostate and gut. CCL20 is implicated in the formation and function of mucosal lymphoid tissues via chemoattraction of lymphocytes and dendritic cells towards the epithelial cells surrounding these tissues. CCL20 elicits its effects on its target cells by binding and activating the chemokine receptor CCR6.

“Chemokine (C-X-C motif) ligand 13” (“CXCL13”) (“B lymphocyte chemoattractant” [“BLC”]; “B cell-attracting chemokine 1” [“BCA-1”]), is a protein ligand and is a small chemokine belonging to the CXC chemokine family. It is selectively chemotactic for B cells belonging to both the B-1 and B-2 subsets and elicits its effects by interacting with chemokine receptor CXCR5. CXCL13 and its receptor CXCR5 control the organization of B cells within follicles of lymphoid tissues and is expressed highly in the liver, spleen, lymph nodes, and gut of humans. In T-cells, CXCL13 expression is thought to reflect a germinal center origin of the T cell, particularly a subset of T cells called follicular B helper T cells (or TFH cells).

In some embodiments, the hydrogel composition comprises a chemokine. In some embodiments, the chemokine comprises CCL20 or CXCL13.

In some embodiments, the hydrogel composition comprises a protein, a peptide, or an oligopeptide.

“Proteins” are large biomolecules and macromolecules that are comprised of one or more long chains of amino acid residues. Proteins differ from one another primarily in their sequence of amino acids, which is dictated by the nucleotide sequence of their genes, and which usually results in protein folding into a specific 3D structure that determines its activity. A linear chain of amino acid residues is called a “polypeptide.” A protein contains at least one long polypeptide. The individual amino acid residues are bonded together by peptide bonds and adjacent amino acid residues. The sequence of amino acid residues in a protein is defined by the sequence of a gene, which is encoded in the genetic code. In general, the genetic code specifies 20 standard amino acids; but in certain organisms the genetic code can include selenocysteine and—in certain archaea—pyrrolysine. Shortly after or even during synthesis, the residues in a protein are often chemically modified by post-translational modification, which alters the physical and chemical properties, folding, stability, activity, and ultimately, the function of the proteins. Some proteins have non-peptide groups attached, which can be called prosthetic groups or cofactors. Proteins can also work together to achieve a particular function, and they often associate to form stable protein complexes. A polypeptide that contains more than approximately fifty amino acids is known as a protein. “Peptides” are short polypeptides, typically containing more than 10-15 residues and fewer than 20-30 residues. “Oligopeptides” typically contain up to 10-15 residues.

### Antibodies, Antigen-Binding Sites, and Other Immunogens

In some embodiments, the hydrogel composition further comprises a pharmaceutical composition, a cytokine or a chemokine, a modulating agent, a targeting agent, or a stabilizing agent.

In some embodiments, the modulating agent comprises an inhibitor or an activator (e.g., a modified CAR-T activating domain. In some embodiments, the modulating agent comprises an immunomodulating agent. In some embodiment, the immunomodulating agent comprises an antibody, an antigen-binding domain, or an antigen.

In some embodiments, the targeting agent comprises an antibody, an antigen-binding domain, or an antigen.

Cytotoxic T-lymphocyte-associated protein 4 (CTLA4 or CTLA-4; CD152 [cluster of differentiation 152]) is a protein receptor that functions as an immune checkpoint and downregulates immune responses. CTLA-4 is constitutively expressed in regulatory T cells, but only upregulated in conventional T cells after activation. This situation is particularly observed in some cancers. For example, it can serve as an “off” switch when bound to CD80 or CD86 on the surface of antigen-presenting cells.

In some embodiments, the hydrogel composition comprises an anti-CTLA-4 antibody or an anti-CTLA-4-antigen-binding domain. In some embodiments, the hydrogel composition comprises cytotoxic T-lymphocyte-associated protein 4 (CTLA-4).

Programmed cell death protein 1 (PD-1; cluster of differentiation 279 [CD279]), a cell surface protein, a member of the immunoglobulin superfamily, is expressed on T cells and pro-B cells and promotes self-tolerance by suppressing T-cell inflammatory activity, preventing autoimmune diseases, but also inhibiting the immune system from killing cancer cells. Programmed cell death protein 1 (PD-1; cluster of differentiation 279 [CD279]), is a cell surface protein that has a role in regulating the immune system's response to the cells of the human body by down-regulating the immune system and promoting self-tolerance by suppressing T cell inflammatory activity. An immune checkpoint protein, PD-1 promotes apoptosis of antigen-specific T-cells in lymph nodes and reduces apoptosis in regulatory T-cells (Tregs). PD-1 is a cell surface receptor that belongs to the immunoglobulin superfamily and is expressed on T cells and pro-B cells. PD-1 binds two ligands, programmed death-ligand 1 (PD-L1) and programmed death-ligand 2 (PD-L2).

In some embodiments, the hydrogel composition comprises an anti-PD-1 antibody or an anti-PD-1-antigen-binding domain. In some embodiments, the hydrogel composition comprises programmed cell death protein 1 (PD-1).

Programmed death-ligand 1 (PD-L1) is highly expressed on the surface of cells of some types of cancers, including, but not limited to, melanoma, bladder cancer, and gastric cancer. As a result, PD-1 inhibitors block PD-1 and lower immune system activation when attacking tumors.

In some embodiments, the hydrogel composition comprises an anti-PD-L1 antibody or an anti-PD-L1-antigen-binding domain. In some embodiments, the hydrogel composition comprises programmed death-ligand1 (PD-L1).

In some embodiments, the hydrogel composition comprises an anti-RANKL agent. In some embodiments, the anti-RANKL agent comprises an anti-RANKL antibody or antigen-binding domain.

In certain embodiments, the hydrogel composition comprises an anti-PD-1 antibody or an anti-PD-1 antigen-binding domain in combination with either (a) an anti-PD-L1 antibody or an anti-PD-L1 antigen-binding domain, (b) an anti-CTLA-4 antibody or an anti-CTLA-4 antigen-binding domain, or (c) an anti-RANKL antibody or an anti-RANKL antigen-binding domain.

In certain embodiments, the hydrogel composition comprises an immunogen, such as an antibody or other antigen-binding site moiety or an affinity reagent.

An “antibody,” an “antigen-binding site” or an “affinity reagent,” is a molecule that binds to an antigen or receptor or another molecule. In some embodiments, an antigen-binding site is a molecule that specifically binds to an antigen or receptor or other molecule. In certain embodiments, some or all of the immunogen is composed of amino acids (including natural, non-natural, and modified amino acids), nucleic acids, or saccharides. In certain embodiments, an antibody-binding site or affinity reagent or other immunogens a small molecule. In certain embodiments, antibodies specifically bind to molecules or targets, such as a cell surface antigen, a cell surface receptor, or other cell surface molecule. Antibodies are discussed in more detail, infra.

In certain embodiments, the hydrogel composition comprises an immunogen, for example, an immunogenic antigen. An immunogen is an antigen or any substance that may be specifically bound by components of the immune system (e.g., antibody, lymphocytes). An immunogen is capable of inducing humoral or cell-mediated immune response rather than immunological tolerance. For example, the immunogen may be selected from the group consisting of keyhole limpet hemocyanin (KLH), concholepas concholepas hemacyanin (CCH), bovine serum albumin (BSA), and ovalbumin (OVA). Further information may be found in Chen et al. (2013) Immunity 39:1-10; and Chen et al. (2012) Clin Cancer Res. 18:6580-6587 (both incorporated by reference).

In some embodiments, the hydrogel composition comprises an antibody (including, e.g., IgG or IgM based or their truncated forms). In some embodiments, the antibody is attached to a pharmaceutical composition; in some embodiments, the antibody is attached to the chemokine or other cytokine; in some embodiment, the antibody is attached to a nucleic acid (e.g., an mRNA, an siRNA, an miRNA, an antisense DNA, or a cDNA); in some embodiments, the antibody is attached to a protein or peptide. In some embodiments, it is attached to another element in the composition, either directly or indirectly (e.g., via a linker). In some embodiments, the antibody targets a tumor cell or a cancer cell. Essentially, the antibody specifically targets a tumor or cancer antigen of interest on a tumor cell of interest or a cancer cell of interest, respectively. The antibody binds to the antigen on the surface of the tumor cell or cancer cell. The specific targeting of the tumor cell or the cancer cell reduces side effects. Some embodiments include targeting by an antibody to provide specific discrimination of the target cell for targeting of, e.g., a pharmaceutical composition, a cytokine, a chemokine, a nucleic acid, or a protein or peptide. Some embodiments include targeting by an antibody and a cytokine or chemokine to provide even more specific discrimination of the target cell. Antibody linkers include, but are not limited to disulfides, hydrazones, peptides, or thioethers. In some embodiments, they are cleavable (e.g., peptides), while in other embodiments, they are non-cleavable (e.g., thioethers). Cleavable linkers may be engineered to be enzyme-sensitive. Non-cleavable linkers typically offer increased stability and maintain the drug within the cell. Longer linkers provide greater physical flexibility in the linker region, potentially altering cleavage kinetics.

In some embodiments, the hydrogel composition comprises an antibody-drug conjugate (ADC). An “antibody-drug conjugate” comprises an antibody and a drug, optionally joined by an “ADC linker.” In some embodiments, the ADC comprises an antibody that targets a tumor cell or a cancer cell, and the drug comprises a cytotoxic drug that destroys the tumor cell or the cancer cell. This type of bioconjugate/immunoconjugate combines the targeting capability of a monoclonal antibody with the tumor cell-destroying or the cancer cell-destroying ability of a cytotoxic drug. Essentially, the antibody specifically targets a tumor antigen of interest or cancer antigen of interest on a tumor cell of interest or cancer cell of interest, respectively. The antibody-drug binds to the antigen on the surface of the tumor cell or cancer cell, and in turn, kills the tumor cell or the cancer cell. Some embodiments include targeting by both the ADC and a cytokine or chemokine, thereby providing even more specific discrimination of the target cell. ADC linkers include, but are not limited to disulfides, hydrazones, peptides, or thioethers. In some embodiments, they are cleavable (e.g., peptides), while in other embodiments, they are non-cleavable (e.g., thioethers). Cleavable ADC linkers may be engineered to be enzyme-sensitive. Non-cleavable ADC linkers typically offer increased stability and maintain the drug within the cell. Longer ADC linkers provide greater physical flexibility in the ADC linker region, potentially altering cleavage kinetics.

In some embodiments, an antigen-binding domain may be comprised of proteinaceous structures other than antibodies that are able to bind to protein targets specifically, including but not limited to avimers, ankyrin repeats and adnectins, and other such proteins with domains that can be evolved to generate specific affinity for antigens, collectively referred to as “antibody-like molecules.” Modifications of proteinaceous affinity reagents through the incorporation of unnatural amino acids during synthesis may be used to improve their properties. Such modifications may have several benefits, including the addition of chemical groups that facilitate subsequent conjugation reactions. In some embodiments, the antigen-binding domain may be a peptide. In some embodiments, the peptide chain is a bispecific peptide. Peptides can readily be made and screened to create affinity reagents that recognize and bind to macromolecules such as proteins.

Bispecific affinity reagents may be constructed by separate synthesis and expression of the first and second affinity reagents. A polypeptide bispecific reagent can be expressed as two separately encoded chains that are linked by disulfide bonds during production in the same host cell, such as, for example, a single chain variable fragment (scFv), a “bispecific single chain variable fragment” (“diabody”; “dibody”), or a trispecific single chain variable fragment (“triabody”; “tribody”). Similarly, standard and widely used solid-phase peptide synthesis technology can be used to synthesize peptides, and chimeric bispecific peptides are well known in the art. A bispecific peptide strategy may be used to combine the first and second first and second affinity reagents in a single peptide chain. Alternatively, polypeptide chains or peptide chains can be expressed/synthesized separately, purified and then conjugated chemically to produce the bispecific affinity reagents useful in the compositions and methods described herein. Many different formats of antibodies may be used. Whole antibodies, F(ab′)2, F(ab′), scFv, as well as smaller Fab and single-domain antibody fragments may all be used to create the first and second affinity reagents. Following their expression and purification, the targeting agents can be chemically conjugated to the protein vehicle. Many conjugation chemistries may be used to effect this conjugation, including homofunctional or heterofunctional linkers that yield ester, amide, thioether, carbon-carbon, or disulfide linkages.

In some embodiments, a peptide aptamer is included. A peptide aptamer is a peptide molecule that specifically binds to a target protein and interferes with the functional ability of that target protein. Peptide aptamers consist of a variable peptide loop attached at both ends of a protein scaffold. Such peptide aptamers can often have a binding affinity comparable to that of an antibody (nanomolar range). Due to the highly selective nature of peptide aptamers, they can be used not only to target a specific protein, but also to target specific functions of a given protein (e.g., a signaling function). Peptide aptamers are usually prepared by selecting the aptamer for its binding affinity with the specific target from a random pool or library of peptides. Peptide aptamers can be isolated from random peptide libraries by yeast two-hybrid screens. They can also be isolated from phage libraries or chemically generated peptides/libraries.

In certain embodiments, the hydrogel composition further comprises an adjuvant. In certain embodiments, the hydrogel comprises an immunogen and an adjuvant: recruiting of professional antigen-presenting cells (APCs) to the site of antigen exposure; increasing the delivery of antigens by delayed/slow release (depot generation); immunomodulation by cytokine production (selection of Th1 or Th2 response); inducing T-cell response (prolonged exposure of peptide-MHC complexes [signal 1] and stimulation of expression of T-cell-activating co-stimulators [signal 2] on the APCs' surface) and targeting (e.g. carbohydrate adjuvants which target lectin receptors on APCs). Examples of adjuvants include, but are not limited to Freund's Complete Adjuvant, lipopolysaccharides, muramyldipeptide from TB, synthetic polynucleotides, aluminum hydroxide, aluminum phosphate, cytokines, and squalene.

As used herein, an “antibody,” an “antigen-binding site” or an or “affinity reagent,” is a molecule that binds to an antigen or receptor or another molecule. In some embodiments, an antibody, an antigen-binding site, an affinity reagent, or other immunogen is a molecule that specifically binds to an antigen or receptor or other molecule. In certain embodiments, some or all of an antibody, antigen-binding site, affinity reagent, or immunogen is composed of amino acids (including natural, non-natural, and modified amino acids), nucleic acids, or saccharides. In certain embodiments, an antigen-binding site, affinity reagent, or immunogen is a small molecule. In certain embodiments, antibodies specifically bind to molecules or targets, such as a cell surface antigen, a cell surface receptor, or other cell surface molecule.

As used herein, the term “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains VL and CL, and each heavy chain comprising immunoglobulin domains VH, C-gamma-1 (Cγ1), C-gamma-2 (Cγ2), and C-gamma-3 (Cγ3). In each pair, the light and heavy chain variable regions (VL and VH) are together responsible for binding to an antigen, and the constant regions (CL, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains VH, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains including but not limited to VH, Cγ1, Cγ2, Cγ3, VL, and CL.

Depending on the amino acid sequence of the constant domain of their heavy chains, intact antibodies can be assigned to different “classes”. There are five-major classes (isotypes) of intact antibodies: IgA, IgD, IgE, IgG, and IgM, and several of these may be further divided into “subclasses”, e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are known to one skilled in the art.

As used herein, the term “immunoglobulin G” or “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. As used herein, the term “modified immunoglobulin G” refers to a molecule that is derived from an antibody of the “G” class. As used herein, the term “antibody” refers to a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA isotypes or classes, respectively.

The term “antibody” is meant to include full-length antibodies and may refer to a natural antibody from any organism, an engineered antibody, or an antibody generated recombinantly for experimental, therapeutic, or other purposes as further defined below. Furthermore, full-length antibodies comprise conjugates as described and exemplified herein. As used herein, the term “antibody” comprises monoclonal antibodies (mAb, moAb) and polyclonal antibodies. Antibodies can be antagonists, agonists, neutralizing, inhibitory, or stimulatory. Specifically included within the definition of “antibody” are full-length antibodies described and exemplified herein. By “full length antibody” herein is meant the structure that constitutes the natural biological form of an antibody, including variable and constant regions.

A “monoclonal antibody” (mAb, moAb) is an antibody made by cloning a unique white blood cell. All subsequent antibodies derived this way trace back to a unique parent cell. Monoclonal antibodies can have monovalent affinity, binding only to the same epitope (the part of an antigen that is recognized by the antibody). In contrast, “polyclonal antibodies” bind to multiple epitopes and are usually made by several different antibody secreting plasma cell lineages. “Bispecific monoclonal antibodies” can also be engineered, by increasing the therapeutic targets of one monoclonal antibody to two epitopes.

The “variable region” of an antibody contains the antigen binding determinants of the molecule, and thus determines the specificity of an antibody for its target antigen. The variable region is so named because it is the most distinct in sequence from other antibodies within the same isotype. The majority of sequence variability occurs in the complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens.

Furthermore, antibodies may exist in a variety of other forms including, for example, Fv, Fab, and (Fab′)2, as well as bi-functional (i.e., bi-specific) hybrid antibodies (e.g., Lanzavecchia et al., (1987) Eur. J. Immunol. 17:105) and in single chains (e.g., Huston et al. (1988) Proc. Natl. Acad. Sci. U.S.A. 85:5879-5883 and Bird et al. (1988) Science 242:423-426 (and related Erratum (1989) Science 244:409), which are incorporated herein by reference). (See, generally, Hood et al., “Immunology”, Benjamin, N.Y., 2nd ed. (1984), and Hunkapiller et al. 1 (1986) Nature 323:15-16). Bispecific antibodies are a technique for creating a single polypeptide that binds to two different determinants. Bispecific antibodies may be made in many different formats, including but not limited to quadroma, F(ab′)2, tetravalent, heterodimeric scFv, bispecific scFv, tandem scFv, diabody and minibody formats, or scFvs appended to or recombinantly fused with whole antibodies.

The term “epitope” as used herein refers to a region of the antigen that binds to the antibody or antigen-binding fragment. It is the region of an antigen recognized by a first antibody wherein the binding of the first antibody to the region prevents binding of a second antibody or other bivalent molecule to the region. The region encompasses a particular core sequence or sequences selectively recognized by a class of antibodies. In general, epitopes are comprised by local surface structures that can be formed by contiguous or noncontiguous amino acid sequences.

As used herein, the terms “selectively recognizes”, “selectively bind” or “selectively recognized” mean that binding of the antibody, antigen-binding fragment or other bivalent molecule to an epitope is at least 2-fold greater, preferably 2-5 fold greater, and most preferably more than 5-fold greater than the binding of the molecule to an unrelated epitope or than the binding of an antibody, antigen-binding fragment or other bivalent molecule to the epitope, as determined by techniques known in the art and described herein, such as, for example, ELISA or cold displacement assays.

As used herein, the term “Fc domain” encompasses the constant region of an immunoglobulin molecule. The Fc region of an antibody interacts with a number of Fc receptors and ligands, imparting an array of important functional capabilities referred to as effector functions, as described herein. For IgG the Fc region comprises Ig domains CH2 and CH3. An important family of Fc receptors for the IgG isotype are the Fc gamma receptors (FcγRs). These receptors mediate communication between antibodies and the cellular arm of the immune system.

As used herein, the term “Fab domain” encompasses the region of an antibody that binds to antigens. The Fab region is composed of one constant and one variable domain of each of the heavy and the light chains.

In one embodiment, the term “antibody” or “antigen-binding fragment” respectively refer to intact molecules as well as functional fragments thereof, such as Fab, a scFv-Fc bivalent molecule, F(ab′)2, and Fv that are capable of specifically interacting with a desired target. In some embodiments, the antigen-binding fragments comprise:
(1) Fab, the fragment which contains a monovalent antigen-binding fragment of an antibody molecule, which can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)2 is a dimer of two Fab′ fragments held together by two disulfide bonds;
(4) Fv, a genetically engineered fragment containing the variable region of the light chain and the variable region of the heavy chain expressed as two chains;
(5) Single chain antibody (“SCA”), a genetically engineered molecule containing the variable region of the light chain and the variable region of the heavy chain, linked by a suitable polypeptide linker as a genetically fused single chain molecule; and
(6) scFv-Fc, is produced in one embodiment, by fusing single-chain Fv (scFv) with a hinge region from an immunoglobulin (Ig) such as an IgG, and Fc regions.

In some embodiments, an antibody provided herein is a monoclonal antibody. In some embodiments, the antigen-binding fragment provided herein is a single chain Fv (scFv), a diabody, a tri (a) body, a di- or tri-tandem scFv, a scFv-Fc bivalent molecule, a Fab, Fab′, Fv, F(ab′)2 or an antigen binding scaffold (e.g., affibody, monobody, anticalin, DARPin, Knottin, etc.). “Affibodies” are small proteins engineered to bind to a large number of target proteins or peptides with high affinity, often imitating monoclonal antibodies, and are antibody mimetics.

As used herein, the terms “bivalent molecule” or “BV” refer to a molecule capable of binding to two separate targets at the same time. The bivalent molecule is not limited to having two and only two binding domains and can be a polyvalent molecule or a molecule comprised of linked monovalent molecules. The binding domains of the bivalent molecule can selectively recognize the same epitope or different epitopes located on the same target or located on a target that originates from different species. The binding domains can be linked in any of a number of ways including, but not limited to, disulfide bonds, peptide bridging, amide bonds, and other natural or synthetic linkages known in the art (Spatola et al., “Chemistry and Biochemistry of Amino Acids, Peptides and Proteins,” B. Weinstein, eds., Marcel Dekker, New York, p. 267 (1983); Morley (1980) Trends Pharm Sci. 463-468; Hudson et al. (1979) Int. J. Pept. Prot. Res. 14:177-185; Spatola et al. (1986) Life Sci. 38:1243-1249; Hann (1982) J. Chem. Soc. Perkin Trans. I 307-314; Almquist et al. (1980) J. Med. Chem. 23:1392-1398; Jennings-White et al. (1982) Tetrahedron Lett. 23:2533-2534; Szelke et al., European Application EP 45665 B1; Szelke et al. U.S. Pat. No. 4,424,407; Chemical Abstracts 97, 39405 (1982); Holladay et al. (1983) Tetrahedron Lett. 24:4401-4404; and Hruby (1982) Life Sci. 31:189-199).

As used herein, the terms “binds” or “binding” or grammatical equivalents, refer to compositions having affinity for each other. “Specific binding” is where the binding is selective between two molecules. A particular example of specific binding is that which occurs between an antibody and an antigen. Typically, specific binding can be distinguished from non-specific when the dissociation constant (KD) is less than about 1×10-5 M or less than about 1×10-6 M or 1×10-7 M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In addition to antibody sequences, an antibody according to the present invention may comprise other amino acids, e.g., forming a peptide or polypeptide, such as a folded domain, or to impart to the molecule another functional characteristic in addition to ability to bind antigen. For example, antibodies of the invention may carry a detectable label, such as fluorescent or radioactive label, or may be conjugated to a toxin (such as a holotoxin or a hemitoxin) or an enzyme, such as beta-galactosidase or alkaline phosphatase (e.g., via a peptidyl bond or linker).

In one embodiment, an antibody of the invention comprises a stabilized hinge region. The term “stabilized hinge region” will be understood to mean a hinge region that has been modified to reduce Fab arm exchange or the propensity to undergo Fab arm exchange or formation of a half-antibody or a propensity to form a half-antibody. “Fab arm exchange” refers to a type of protein modification for human immunoglobulin, in which a human immunoglobulin heavy chain and attached light chain (half-molecule) is swapped for a heavy-light chain pair from another human immunoglobulin molecule. Thus, human immunoglobulin molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half-antibody” forms when a human immunoglobulin antibody dissociates to form two molecules, each containing a single heavy chain and a single light chain. In one embodiment, the stabilized hinge region of human immunoglobulin comprises a substitution in the hinge region.

In one embodiment, the term “hinge region” as used herein refers to a proline-rich portion of an immunoglobulin heavy chain between the Fc and Fab regions that confers mobility on the two Fab arms of the antibody molecule. It is located between the first and second constant domains of the heavy chain. The hinge region includes cysteine residues which are involved in inter-heavy chain disulfide bonds. In one embodiment, the hinge region includes cysteine residues which are involved in inter-heavy chain disulfide bonds.

In some embodiments, the present invention comprises a first component protein comprising a first binding pair partner and a second component protein comprising a second binding pair partner, wherein the binding pair partners comprise two protein moieties that form a heterodimer.

A “dimer” is a macromolecular complex formed by two macromolecules, usually proteins (or portions thereof) or nucleic acids (or portions thereof). A “homodimer” is formed by two identical macromolecules (“homodimerization”), while a “heterodimer” is formed by two distinct macromolecules (“heterodimerization”). Many dimers are non-covalently linked, but some (e.g., NEMO homodimers) can link via, e.g., disulfide bonds. Some proteins comprise regions specialized for dimerization, known as “dimerization domains.” In some instances, a truncated protein containing or comprising a dimerization domain (or two truncated proteins containing or comprising corresponding dimerization domains) may be able to interact in the absence of one or both complete protein sequence(s). Similarly, a fusion protein comprising a dimerization domain (or two fusion proteins comprising corresponding dimerization domains) may be able to interact in the absence of one or both complete protein sequence(s). Mutations to these domains may increase, or alternatively reduce, the formation of a dimer. Examples of macromolecules that can form dimers include, but are not limited to, proteins, nucleic acids, antibodies, receptor tyrosine kinases, proteins with leucine zippers, peptide Velcro, nuclear receptors, 14-3-3 proteins, G proteins, G protein-coupled receptors, transcription factors, kinesin, triosephosphate isomerase (TIM), alcohol dehydrogenase, Toll-like receptors, fibrinogen, tubulin, some glycoproteins, and some clotting factors. Additional examples of particular pairs include, but are not limited to, c-Jun/c-Fos, RelA (or c-Rel or RelB)/p50 (or p51) (Rel/NF-kappaB), AP-1, C/EBP, ATF/CREB, c-Myc, and NF-1.

The cell surface antigen may be any cell surface molecule that undergoes internalization, such as a protein, sugar, lipid head group or other antigen on the cell surface. Examples of cell surface antigens useful in the context of the invention include but are not limited to the transferrin receptor type 1 and 2, the EGF receptor (e.g., IMC-225), HER2/Neu (e.g., tastuzumab or pertuzumab), VEGF receptors, integrins, CD33, CD19, CD20, CD22, CD4 and the asialoglycoprotein receptor.

In certain embodiments, the construct relates to any of the compositions described herein, wherein the antibody is an anti-PD-1 antibody or wherein the antigen-binding site is an anti-PD-1 antigen-binding site. In certain embodiments, the construct relates to any of the compositions described herein, wherein the antibody is an anti-PD-1 antibody or wherein the antigen-binding site is an anti-PD-1 antigen-binding site, each either alone or in combination with either (a) an anti-PD-L1 antibody or an anti-PD-L1 antigen-binding site or (b) an anti-CTLA-4 antibody or an anti-CTLA-4 antigen binding site.

Antibodies for use in the invention may be raised through any conventional method, such as through injection of immunogen into mice and subsequent fusions of lymphocytes to create hybridomas. Such hybridomas may then be used either (a) to produce antibody directly, which is purified and used for chemical conjugation to create a bispecific antibody, or (b) to clone cDNAs encoding antibody fragments for subsequent genetic manipulation. To illustrate one method employing the latter strategy, mRNA is isolated from the hybridoma cells, reverse-transcribed into cDNA using antisense oligo-dT or immunoglobulin gene-specific primers and cloned into a plasmid vector. Clones are sequenced and characterized. They may then be engineered according to standard protocols to combine the heavy and light chains of each antibody, separated by a short peptide linker, into a bacterial or mammalian expression vector as previously described to produce a recombinant bispecific antibody, which are then expressed and purified according to well-established protocols in bacteria or mammalian cells. Antibodies, or other proteinaceous affinity molecules or targeting agents such as peptides, may also be created through display technologies that allow selection of interacting affinity reagents through the screening of very large libraries of, for example, immunoglobulin domains or peptides expressed by bacteriophage. Antibodies may also be humanized through grafting of human immunoglobulin domains or made from transgenic mice or bacteriophage libraries that have human immunoglobulin genes/cDNAs.

In some embodiments, a nucleic acid aptamer is included. Nucleic acid aptamers are nucleic acid oligomers that bind other macromolecules specifically; such aptamers that bind specifically to other macromolecules can be readily isolated from libraries of such oligomers by technologies such as SELEX.

In some embodiments, an oligosaccharide is included. Certain oligosaccharides are known ligands for certain extracellular or cell surface receptors.

In one embodiment, the antibody or antigen-binding fragment binds its target with a KD of 0.1 nM-10 mM. In one embodiment, the antibody or antigen-binding fragment binds its target with a KD of 0.1 nM-1 mM. In one embodiment, the antibody or antigen-binding fragment binds its target with a KD within the 0.1 nM range. In one embodiment, the antibody or antigen-binding fragment binds its target with a KD of 0.1-2 nM. In another embodiment, the antibody or antigen-binding fragment binds its target with a KD of 0.1-1 nM. In another embodiment, the antibody or antigen-binding fragment binds its target with a KD of 0.05-1 nM. In another embodiment, the antibody or antigen-binding fragment binds its target with a KD of 0.1-0.5 nM. In another embodiment, the antibody or antigen-binding fragment binds its target with a KD of 0.1-0.2 nM.

In some embodiments, the antibody or antigen-binding fragment thereof provided herein comprises a modification. In another embodiment, the modification minimizes conformational changes during the shift from displayed to secreted forms of the antibody or antigen-binding fragment. It is to be understood by a skilled artisan that the modification can be a modification known in the art to impart a functional property that would not otherwise be present if it were not for the presence of the modification. Encompassed are antibodies which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

In some embodiments, the modification is one as further defined herein below. In some embodiments, the modification is a N-terminus modification. In some embodiments, the modification is a C-terminal modification. In some embodiments, the modification is an N-terminus biotinylation. In some embodiments, the modification is a C-terminus biotinylation. In some embodiments, the secretable form of the antibody or antigen-binding fragment comprises an N-terminal modification that allows binding to an Immunoglobulin (Ig) hinge region. In some embodiments, the Ig hinge region is from but is not limited to, an IgA hinge region. In some embodiments, the secretable form of the antibody or antigen-binding fragment comprises an N-terminal modification that allows binding to an enzymatically biotinylatable site. In some embodiments, the secretable form of the antibody or antigen-binding fragment comprises a C-terminal modification that allows binding to an enzymatically biotinylatable site. In some embodiments, biotinylation of said site functionalizes the site to bind to any surface coated with streptavidin, avidin, avidin-derived moieties, or a secondary reagent.

It will be appreciated that the term “modification” can encompass an amino acid modification such as an amino acid substitution, insertion, and/or deletion in a polypeptide sequence.

In one embodiment, a variety of radioactive isotopes are available for the production of radioconjugate antibodies and other proteins and can be of use in the methods and compositions provided herein. Examples include, but are not limited to, At211, Cu64, I131, I125, Y90, Re186, Re188, Sm153, Bi212, P32, Zr89, F-18, I-124 and radioactive isotopes of Lu. In a further embodiment, the amino acid sequences of the invention may be homologues, variants, isoforms, or fragments of the sequences presented. The term “homolog” as used herein refers to a polypeptide having a sequence homology of a certain amount, namely of at least 70%, e.g., at least 80%, 90%, 95%, 96%, 97%, 98%, 99% of the amino acid sequence it is referred to. Homology refers to the magnitude of identity between two sequences. Homolog sequences have the same or similar characteristics, in particular, have the same or similar property of the sequence as identified. The term ‘variant’ as used herein refers to a polypeptide wherein the amino acid sequence exhibits substantially 70, 80, 95, or 99% homology with the amino acid sequence as set forth in the sequence listing. It should be appreciated that the variant may result from a modification of the native amino acid sequences, or by modifications including insertion, substitution or deletion of one or more amino acids. The term “isoform” as used herein refers to variants of a polypeptide that are encoded by the same gene, but that differ in their isoelectric point (pI) or molecular weight (MW), or both. Such isoforms can differ in their amino acid composition (e.g., as a result of alternative splicing or limited proteolysis) and in addition, or in the alternative, may arise from differential post-translational modification (e.g., glycosylation, acylation, phosphorylation deamidation, or sulphation). As used herein, the term “isoform” also refers to a protein that exists in only a single form, i.e., it is not expressed as several variants. The term “fragment” as used herein refers to any portion of the full-length amino acid sequence of protein of a polypeptide of the invention which has less amino acids than the full-length amino acid sequence of a polypeptide of the invention. The fragment may or may not possess a functional activity of such polypeptides.

In an alternate embodiment, enzymatically active toxin or fragments thereof that can be used in the compositions and methods provided herein include, but are not limited, to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

A chemotherapeutic or other cytotoxic agent may be conjugated to the protein, according to the methods provided herein, as an active drug or as a prodrug. The term “prodrug” refers to a precursor or derivative form of a pharmaceutically active substance that is less cytotoxic to tumor cells compared to the parent drug and is capable of being enzymatically activated or converted into the more active parent form. (See, for example Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985.) The prodrugs that may find use with the compositions and methods as provided herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies and Fc fusions of the compositions and methods as provided herein include but are not limited to any of the aforementioned chemotherapeutic.

### Nucleic Acids

In some embodiments, the hydrogel further comprises a composition comprising a therapeutic or biotherapeutic, a modulating agent, a targeting agent, a pharmaceutical composition, or a stabilizing agent.

In some embodiments, the modulating agent comprises a nucleic acid. In some embodiments, the targeting agent comprises a nucleic acid.

In particular, the nucleic acid sequence, amino acid sequence, functional domain, structural domain, gene locus, and other identifying information for the signaling pathway targets described herein are well known in the art.

In some embodiments, the hydrogel composition comprises a nucleic acid (e.g., a deoxyribonucleic acid [DNA] or a ribonucleic acid [RNA]). In some embodiments, the nucleic acid is double-stranded (ds); in some embodiments, the nucleic acid is single-stranded (ss). In some embodiments, the nucleic acid comprises an antisense nucleic acid or a portion thereof. In some embodiments, the nucleic acid comprises an oligonucleotide.

In some embodiments, the nucleic acid comprises a DNA. In some embodiments, the DNA comprises a genomic DNA or a portion thereof or a complementary DNA (cDNA) or a portion thereof. In some embodiments, the DNA is double-stranded; in some embodiments, the DNA is single-stranded. In some embodiments, the nucleic acid comprises an antisense DNA. In some embodiment, the nucleic acid comprises a single-stranded antisense DNA.

In some embodiments, the nucleic acid comprises an RNA. In some embodiments, the RNA comprises a messenger RNA (mRNA), a small interfering RNA (siRNA), or a microRNA (miRNA). In some embodiments, the RNA is double-stranded; in some embodiments, the RNA is single-stranded. In certain embodiments, the hydrogel composition comprises an siRNA moiety comprised of a sense strand and an antisense strand; the sense strand comprising a 3′ end and a 5′ end; and the antisense strand comprising a 3′ end and a 5′ end.

“Antisense” nucleic acids refer to nucleic acids that specifically hybridize (e.g., bind) with a complementary sense nucleic acid, e.g., cellular mRNA and/or genomic DNA, under cellular conditions so as to inhibit expression (e.g., by inhibiting transcription and/or translation). The binding may be by conventional base pair complementarity or, for example, in the case of binding to DNA duplexes, through specific interactions in the major groove of the double helix.

Antisense technology is the process in which an antisense RNA or DNA molecule interacts with a target sense DNA or RNA strand. A sense strand is a 5′ to 3′ mRNA molecule or DNA molecule. The complementary strand, or mirror strand, to the sense is called an antisense. When an antisense strand interacts with a sense mRNA strand, the double helix is recognized as foreign to the cell and will be degraded, resulting in reduced or absent protein production. Although DNA is already a double-stranded molecule, antisense technology can be applied to it, building a triplex formation.

One skilled in the art would appreciate that the terms “complementary” or “complement thereof” are used herein to encompass the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

RNA antisense strands can be either catalytic or non-catalytic. The catalytic antisense strands, also called ribozymes, cleave the RNA molecule at specific sequences. A non-catalytic RNA antisense strand blocks further RNA processing.

Antisense modulation of cells and/or tissue levels of the globulin genes of interest and/or desaturase genes of interest or any combination thereof may be effected by transforming the organism's cells or tissues with at least one antisense compound, including antisense DNA, antisense RNA, a ribozyme, DNAzyme, a locked nucleic acid (LNA) and an aptamer. In some embodiments the molecules are chemically modified. In other embodiments the antisense molecule is antisense DNA or an antisense DNA analog.

Antisense modulation of cells and/or tissue levels of the globulin genes of interest and/or desaturase genes of interest or any combination thereof may be effected by transforming the organism's cells or tissues with at least one antisense compound, including antisense DNA, antisense RNA, a ribozyme, DNAzyme, a locked nucleic acid (LNA), and an aptamer. In some embodiments, the molecules are chemically modified. In other embodiments, the antisense molecule is antisense DNA or an antisense DNA analog.

The term “RNA interference” or “RNAi” refers to the silencing or decreasing of gene expression mediated by small double stranded RNAs. It is the process of sequence-specific, post-transcriptional gene silencing in animals and plants, initiated by inhibitory RNA (iRNA) that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

One of ordinary skill in the art would appreciate that the term RNAi molecule refers to single- or double-stranded RNA molecules comprising both a sense and antisense sequence. For example, the RNA interference molecule can be a double-stranded polynucleotide molecule comprising self-complementary sense and antisense regions, wherein the antisense region comprises complementarity to a target nucleic acid molecule. Alternatively the RNAi molecule can be a single-stranded hairpin polynucleotide having self-complementary sense and antisense regions, wherein the antisense region comprises complementarity to a target nucleic acid molecule or it can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active molecule capable of mediating RNAi.

RNAi refers to the introduction of homologous double-stranded RNA (dsRNA) to target a specific gene product, resulting in post transcriptional silencing of that gene. This phenomenon was first reported in Caenorhabditis elegans by Guo and Kemphues (1995, Cell, 81 (4): 611-620) and subsequently Fire et al. (1998, Nature 391:806-811) discovered that it is the presence of dsRNA, formed from the annealing of sense and antisense strands present in the in vitro RNA preps, that is responsible for producing the interfering activity.

In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs.

The dsRNA used to initiate RNAi, may be isolated from native source or produced by known means, e.g., transcribed from DNA. Plasmids and vectors for generating RNAi molecules against target sequence are now readily available from commercial sources.

The dsRNA can be transcribed from the vectors as two separate strands. In other embodiments, the two strands of DNA used to form the dsRNA may belong to the same or two different duplexes in which they each form with a DNA strand of at least partially complementary sequence. When the dsRNA is thus produced, the DNA sequence to be transcribed is flanked by two promoters, one controlling the transcription of one of the strands, and the other that of the complementary strand. These two promoters may be identical or different. Alternatively, a single promoter can derive the transcription of single-stranded hairpin polynucleotide having self-complementary sense and antisense regions that anneal to produce the dsRNA.

One skilled in the art would appreciate that the terms “promoter element,” “promoter,” or “promoter sequence” may encompass a DNA sequence that is located at the 5′ end (i.e., precedes) the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases. There is no upper limit on the length of the dsRNA that can be used. For example, the dsRNA can range from about 21 base pairs (bp) of the gene to the full-length of the gene or more.

In some embodiments, the hydrogel composition comprises a small interfering RNA (siRNA). The siRNA moiety may further include a guanosine at the 5′-end.

The sense and/or antisense strands of the siRNA moiety may equal to or less than 30, 25, 24, 23, 22, 21, 20, 19, 18 or 17 nucleotides in length. An siRNA moiety may include one or more overhangs. For example, the siRNA moiety may include one or two 3′ overhangs of 2-3 nucleotides. In certain embodiments, the invention relates to any of the compositions described herein, wherein the siRNA moiety is composed of 21-nt sense and 21-nt antisense strands, paired in a manner to have a 19-nucleotide duplex region and a 2-nt 3′ overhang at each 3′ terminus. In certain embodiments, the invention relates to any of the compositions describe herein, wherein the 2-nt 3′ overhang is either UU or dTdT. Symmetric 3′-overhangs ensure that the sequence-specific endonuclease complexes (siRNPs) are formed with approximately equal ratios of sense and antisense target RNA cleaving siRNPs. The 3′-overhang in the sense strand provides no contribution to recognition as it is believed the antisense siRNA strand guides target recognition. Therefore, the UU or dTdT 3′-overhang of the antisense sequences is complementary to the target mRNA but the symmetrical UU or dTdT 3′-overhang of the sense siRNA oligo does not need to correspond to the mRNA. The use of deoxythymidines in both 3′-overhangs may increase nuclease resistance, although siRNA duplexes with either UU or dTdT overhangs work equally well. 2′-Deoxynucleotides in the 3′ overhangs are as efficient as ribonucleotides, but are often cheaper to synthesize.

The targeted region in the mRNA, and hence the sequence in the siRNA duplex, are chosen using the following guidelines. The open reading frame (ORF) region from the cDNA sequence is recommended for targeting, preferably at least 50 to 100 nucleotides downstream of the start codon, most preferably at least 75-100. Both the 5′ and 3′ untranslated regions (UTRs) and regions near the start codon are not recommended for targeting as these may be richer in regulatory protein binding sites. UTR-binding proteins and/or translation initiation complexes may interfere with binding of the siRNP endonuclease complex.

The sequence of the mRNA or cDNA is searched seeking the sequence AA(N19)TT. Sequences with approximately 50% G/C-content (30% to 70%) are used. If no suitable sequences are found, the search is extended to sequences AA(N21). The sequence of the sense siRNA corresponds to 5′-(N19) dTdT-3′ or N21, respectively. In the latter case, the 3′ end of the sense siRNA is converted to dTdT. The rationale for this sequence conversion is to generate a symmetric duplex with respect to the sequence composition of the sense and antisense 3′ overhangs. It is believed that symmetric 3′ overhangs help to ensure that the siRNPs are formed with approximately equal ratios of sense and antisense target RNA-cleaving siRNPs. The modification of the overhang of the sense sequence of the siRNA duplex is not expected to affect targeted mRNA recognition, as the antisense siRNA strand glides target recognition.

If the target mRNA does not contain a suitable AA(N21) sequence, it is recommended to search for NA(N21) The sequence of the sense and antisense strand may still be synthesized as 5′ (N19) TT as the sequence of the 3′ most nucleotide of the antisense siRNA does not appear to contribute to specificity.

It is further recommended to search the selected siRNA sequence against EST libraries in appropriate databases (e.g., NCBI BLAST database search) to ensure that only one gene is targeted.

The appropriately designed siRNAs are either obtained from commercial sources (such as DHARMACON RESEARCH™, Lafayette, Colo.; XERGON™, Huntsville, Ala.; AMBION™, Austin, Tex.) or chemically synthesized used appropriately protected ribonucleoside phosphoramidites and a conventional DNA/RNA synthesizer according to standard protocols. The RNA oligonucleotides are 2′-deprotected, desalted and the two strands annealed, according to manufacturer's specifications or conventional protocols, depending on how the siRNAs are obtained. All handling steps are conducted under strict sterile, RNase-free conditions.

In some embodiments, a nucleic acid aptamer is included. Nucleic acid aptamers are nucleic acid oligomers that bind other macromolecules specifically; such aptamers that bind specifically to other macromolecules can be readily isolated from libraries of such oligomers by technologies such as SELEX. In some embodiments, an oligosaccharide is included. Certain oligosaccharides are known ligands for certain extracellular or cell surface receptors.

### Co-Suppression Molecules

In some embodiments, the hydrogel composition comprises a co-suppression molecule. A co-suppression molecule is another agent capable of down-regulating the expression of a given gene, or a combination thereof. Co-suppression is a post-transcriptional mechanism where both the transgene and the endogenous gene are silenced.

### Enzymatic Nucleic Acid Molecules

In some embodiments, the hydrogel composition comprises an enzymatic nucleic acid molecule. The terms “enzymatic nucleic acid molecule” or “enzymatic oligonucleotide” refers to a nucleic acid molecule which has complementarity in a substrate binding region to a specified gene target and also has an enzymatic activity which is active to specifically cleave target RNA of a given gene, thereby silencing each of the genes. The complementary regions allow sufficient hybridization of the enzymatic nucleic acid molecule to the target RNA and subsequent cleavage. The term enzymatic nucleic acid is used interchangeably with for example, ribozymes, catalytic RNA, enzymatic RNA, catalytic DNA, aptazyme or aptamer-binding ribozyme, catalytic oligonucleotide, nucleozyme, DNAzyme, RNAenzyme. The specific enzymatic nucleic acid molecules described in the instant application are not limiting and an enzymatic nucleic acid molecule of this invention requires a specific substrate binding site which is complementary to one or more of the target nucleic acid regions, and that it have nucleotide sequences within or surrounding that substrate binding site which impart a nucleic acid cleaving and/or ligation activity to the molecule. U.S. Pat. No. 4,987,071 discloses examples of such molecules.

In some embodiments, the hydrogel composition is a DNAzyme molecule. A DNAzyme molecule is another agent capable of down-regulating the expression of a given gene, the DNAzyme molecule being capable of specifically cleaving an mRNA transcript or a DNA sequence of said gene. DNAzymes are single-stranded polynucleotides that are capable of cleaving both single- and double-stranded target sequences. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine: pyrimidine junctions (for review of DNAzymes, see: Khachigian, L. M. (2002) Curr Opin Mol Ther 4, 119-121).

Examples of construction and amplification of synthetic, engineered DNAzymes recognizing single- and double-stranded target cleavage sites are disclosed in U.S. Pat. No. 6,326,174.

### Pharmaceutical Compositions

The present invention further provides a pharmaceutical composition or a pharmaceutically acceptable salt thereof, and a pharmaceutically acceptable carrier.

Disclosed herein is a hydrogel composition comprising a cell, a pharmaceutical composition or a pharmaceutically acceptable salt thereof, an antibody, a chemokine, or a cytokine for use in the treatment of a disease or disorder in a subject in need thereof. In some embodiments, the subject is a human. In one embodiment, the disease or disorder is a cancer, cancer metastasis or a solid tumor.

Disclosed herein is a hydrogel composition comprising a pharmaceutical composition or a pharmaceutically acceptable salt thereof, for use in the treatment of a cancer or a tumor.

In some embodiments, the hydrogel composition comprises a pharmaceutical composition or a pharmaceutically acceptable salt thereof. In some embodiments, the pharmaceutical composition comprises a medicament (e.g., a drug).

In some embodiments, the hydrogel further comprises a composition comprising a therapeutic or biotherapeutic, a modulating agent, a targeting agent, a stabilizing agent, a pharmaceutical or biopharmaceutical composition, or and all the above drug plus a cell a proliferating cell that expends the lymphoid network.

Disclosed herein is a hydrogel composition comprising a pharmaceutical composition or a pharmaceutically acceptable salt thereof, for use for treating, inhibiting, ameliorating, or alleviating a tumor or a cancer in a subject in need thereof. Also disclosed herein is a hydrogel composition comprising a pharmaceutical composition or a pharmaceutically acceptable salt thereof, for use for treating, inhibiting, ameliorating, or alleviating an immune disease or abnormal immune condition. Also disclosed herein is a hydrogel comprises a pharmaceutical composition or a pharmaceutically acceptable salt thereof, for use for treating, inhibiting, ameliorating, or alleviating a disease or abnormal physiological condition.

In some embodiments, the hydrogel composition comprises a pharmaceutical composition or a pharmaceutically acceptable salt thereof, for use in treating, inhibiting, ameliorating, or alleviating a tumor or cancer in a subject in need thereof. In some embodiments, the hydrogel composition comprising a pharmaceutical composition or a pharmaceutically acceptable salt thereof, for use in treating, inhibiting, ameliorating, or alleviating metastasis of a cancer in a subject in need thereof. In some embodiments, the hydrogel composition comprises a pharmaceutical composition or a pharmaceutically acceptable salt thereof, for use for treating, inhibiting, ameliorating, or alleviating an immune disease or abnormal immune condition. In some embodiments, the hydrogel composition comprises a pharmaceutical composition or a pharmaceutically acceptable salt thereof, for use for treating, inhibiting, ameliorating, or alleviating a disease or abnormal physiological condition.

As used herein, “pharmaceutical composition” refers to a therapeutically effective amount of a compound of the present invention, or a pharmaceutically acceptable salt thereof, together with suitable diluents, preservatives, solubilizers, emulsifiers, adjuvant, carriers, or combinations of these. Such compositions are liquids or lyophilized or otherwise dried formulations and include diluents of various buffer content (e.g.; Tris-HCL, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

A “stabilizing agent” (“stabilizer,” “stabilizing excipient”) is a substance used to help the active pharmaceutical ingredient (API) maintain the desirable properties of the product until it is administered to the subject.

A “therapeutically effective amount” as used herein refers to that amount which provides a therapeutic effect for a given indication and administration regimen.

Numerous standard references are available that describe procedures for preparing various compositions or formulations suitable for administration of the compounds of the invention. Examples of methods of making formulations and preparations can be found in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

The mode of administration and dosage form are closely related to the therapeutic amounts of the compounds or compositions which are desirable and efficacious for the given treatment application.

The hydrogel compositions of the invention can be administered to a subject by any method known to a person skilled in the art. These methods include, but are not limited to, orally, parenterally, intravascularly, paracancerally, transmucosally, transdermally, intramuscularly, intranasally, intravenously, intradermally, subcutaneously, sublingually, intraperitoneally, intraventricularly, intracranially, intravaginally, rectally, intratumorally, intra-lymph nodal, or adjacent to a tumor. These methods include any means in which the composition can be delivered to tissue (e.g., needle or catheter).

In some embodiments, the hydrogel composition is administered subcutaneously (SC), interperitoneally (IP), injected intra-lymph nodes, injected intra-tumorally, or injected adjacent to a tumor.

By “adjacent” to a tumor includes, but is not limited to, next to a tumor, adjoining a tumor, or in close proximity to a tumor. By “close proximity” to a tumor is meant that the closest part of the hydrogel composition is from about 1 mm to about 50 mm from a tumor, from about 1 mm to about 45 mm from a tumor, from about 1 mm to about 40 mm from a tumor, from about 1 mm to about 35 mm from a tumor, from about 1 mm to about 30 mm from a tumor, from about 1 mm to about 25 mm from a tumor, from about 1 mm to about 20 mm from a tumor, from about 1 mm to about 15 mm from a tumor, from about 1 mm to about 10 mm from a tumor, from about 1 mm to about 5 mm from a tumor.

Suitable dosage forms include, but are not limited to, subcutaneous (SC), intraperitoneal (IP), intra-tumor or intra-lymph node administration, tumor-adjacent administration and other dosage forms for systemic delivery of active ingredients.

As used herein “pharmaceutically acceptable carriers or diluents” are well known to those skilled in the art. The carrier or diluent may be a solid carrier or diluent for solid formulations, a liquid carrier or diluent for liquid formulations, or mixtures thereof.

Solid carriers/diluents include, but are not limited to, a protein or a salt.

For parenteral formulations, the carrier will usually comprise sterile water, though other ingredients may be included, such as ingredients that aid solubility or for preservation. Injectable suspensions may also be prepared in which case appropriate stabilizing agents may be employed.

Formulations suitable for parenteral administration may comprise a sterile aqueous preparation of the active compound, which, in some embodiments, is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

In addition to the aforementioned ingredients, compositions of this invention may further include one or more ingredient selected from diluents, buffers, flavoring agents, binders, disintegrants, surface active agents, thickeners, lubricants, preservatives (including antioxidants), and the like.

The formulations may be of immediate release, sustained release, delayed-onset release or any other release profile known to one skilled in the art.

The methods of the invention comprise administration of a compound at a therapeutically effective amount. The therapeutically effective amount may include various dosages.

For administration to birds or to mammals, and particularly humans, it is expected that the physician will determine the actual dosage and duration of treatment, which will be most suitable for an individual and can vary with the age, weight, genetics and/or response of the particular individual. In some embodiments, the subject is a mammal (e.g., a human or non-human mammal). In some embodiments, the subject is a human.

The exact dose and regimen of administration of the composition will necessarily be dependent upon the therapeutic or nutritional effect to be achieved and may vary with the particular formula, the route of administration, and the age and condition of the individual subject to whom the composition is to be administered.

A dosage unit of the compounds as used herein may comprise a single compound or mixtures thereof with additional therapeutic agents. A “dose” or “dosage unit” or “unit dosage” of a compound of formula (I) of the invention as measured in milligrams refers to the milligrams of compound of formula (I) present in a preparation, regardless of the form of the preparation.

In some embodiments, a compound of the invention is administered at a dosage of 1-3000 mg per day. In some embodiments, a compound of the invention as described herein is administered at a dosage of 1-1000 mg per day. In some embodiments, a compound of the invention as described herein is administered at a dosage of 1-500 mg per day. In some embodiments, a compound of the invention as described herein is administered at a dosage of 10-500 mg per day. In some embodiments, a compound of the invention as described herein is administered at a dosage of 25-500 mg per day. In some embodiments, a compound of the invention as described herein is administered at a dosage of 50-500 mg per day. In some embodiments, a compound of the invention as described herein is administered at a dosage of 5-250 mg per day. In some embodiments, a compound of the invention as described herein is administered at a dosage of 10-250 mg per day. In some embodiments, a compound of the invention as described herein is administered at a dosage of 20-250 mg per day. In some embodiments, a compound of the invention as described herein is administered at a dosage of 25-250 mg per day. In some embodiments, a compound of the invention as described herein is administered at a dosage of 25-200 mg per day. In some embodiments, a compound of the invention as described herein is administered at a dosage of 25-150 mg per day. In some embodiments, a compound of the invention as described herein is administered at a dosage of 25-125 mg per day. In some embodiments, a compound of the invention as described herein is administered at a dosage of 25-100 mg per day.

In other embodiments, a compound of the invention is administered at a dose of 1-10 mg per day, 3-26 mg per day, 3-60 mg per day, 3-16 mg per day, 3-30 mg per day, 10-26 mg per day, 10-100 mg per day, 15-60 mg per day, 15-100 mg per day, 25-100 mg per day, 50-100 mg per day, 50-200 mg per day, 100-200 mg per day, 100-250 mg per day, 125-300 mg per day, 20-50 mg per day, 5-50 mg per day, 200-500 mg per day, 125-500 mg per day, 500-1000 mg per day, 200-1000 mg per day, 1000-2000 mg per day, 1000-3000 mg per day, 125-3000 mg per day, 2000-3000 mg per day, 300-1500 mg per day or 100-1000 mg per day.

The methods may comprise administering a compound at various dosages. For example, the compound may be administered per day at a dosage of 3 mg, 10 mg, 30 mg, 40 mg, 50 mg, 80 mg, 100 mg, 120 mg, 125 mg, 200 mg, 250 mg, 300 mg, 450 mg, 500 mg, 600 mg, 900 mg, 1000 mg, 1500 mg, 2000 mg, 2500 mg or 3000 mg.

Alternatively, the compound may be administered at a dosage of 0.1 mg/kg/day. The compound may be administered at a dosage between 0.2 to 30 mg/kg/day, or 0.2 mg/kg/day, 0.3 mg/kg/day, 1 mg/kg/day, 3 mg/kg/day, 5 mg/kg/day, 10 mg/kg/day, 20 mg/kg/day, 30 mg/kg/day, 50 mg/kg/day or 100 mg/kg/day.

“Modified-release dosage” is a mechanism that (in contrast to “immediate-release dosage”) delivers a drug with a delay after its administration (delayed-release dosage) or for a prolonged period of time (extended-release [ER, XR, XL] dosage) or to a specific target in the body (targeted-release dosage).

“Sustained-release dosage” forms are dosage forms designed to release (liberate) a drug at a predetermined rate in order to maintain a constant drug concentration for a specific period of time with minimum side effects. This can be achieved through a variety of formulations, including liposomes and drug-polymer conjugates (an example being hydrogels). Sustained release's definition is more akin to a “controlled release” rather than “sustained”.

“Extended-release dosage” (ER) consists of either “sustained-release” (SR) or “controlled-release” (CR) dosage. SR maintains drug release over a sustained period but not at a constant rate. CR maintains drug release over a sustained period at a nearly constant rate.

In some embodiments, the hydrogel composition has a sustained-release (SR) dosage or an extended-release (ER) dosage. In some embodiments, the SR or ER dosage extends at least about 1 day, at least about 2 days, at least about 3 days, at least about 4 days, at least about 5 days, at least about 6 days, at least about 7 days, at least about 8 days, at least about 9 days, at least about 10 days, at least about 2 weeks, at least about 3 weeks, at least about 1 month, at least about 2 months, at least about 3 months, at least about 4 months, at least about 5 months, at least about 6 months, at least about 9 months, or at least about 12 months. In some embodiments, the SR or ER dosage extends at least about 7 days.

As used herein, in some embodiments, the term “alkyl” refers to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms.

In some embodiments, “aryl” refers to monocyclic or polycyclic (e.g., having 2, 3 or 4 fused rings) aromatic hydrocarbons such as, for example, phenyl, naphthyl, anthracenyl, phenanthrenyl, and the like. In some embodiments, an aryl group has from 6 to about 20 carbon atoms. In some embodiments, “aryl” may be optionally substituted at any one or more positions.

In some embodiments, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms. In some embodiments, “heteroaryl” may be optionally substituted at any one or more positions capable of bearing a hydrogen atom.

The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

In some embodiments, “halo” or “halogen” includes fluoro, chloro, bromo, and iodo. A “halogen-substitution” or “halo” substitution designates replacement of one or more hydrogen atoms with F, Cl, Br or I.

In some embodiments, “haloalkyl” refers to an alkyl group having one or more halogen substituents. Example haloalkyl groups include CF₃, C₂F₅, CHF₂, CCl₃, CHCl₂, C₂Cl₅, and the like.

In some embodiments, the term “substituted” refers to the replacement of a hydrogen moiety with a non-hydrogen moiety in a molecule or group. It can refer to “mono-substituted” or “poly-substituted.” The term “mono-substituted” or “poly-substituted” means substituted with one or more than one substituent up to the valence of the substituted group. For example, a mono-substituted group can be substituted with 1 substituent, and a poly-substituted group can be substituted with 2, 3, 4, or 5 substituents. When a list of possible substituents is provided, the substituents can be independently selected from that group.

The term “optionally substituted,” in some embodiments, refers to that the groups in question are either unsubstituted or substituted with one or more of the substituents specified. When the groups in question are substituted with more than one substituent, the substituents may be the same or different. Such other functional groups illustratively include, but are not limited to, amino, hydroxyl, CN, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, CH, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted. In some embodiments, the functional groups are the substituents described herein for any one of variables. Furthermore, when using the terms “independently,” “independently are,” and “independently selected from” mean that the groups in question may be the same or different. Certain of the herein defined terms may occur more than once in the structure, and upon such occurrence each term shall be defined independently of the other.

In each of the foregoing and each of the following embodiments, it is to be understood that the formulas also include any and all hydrates and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

As used herein, the term “solvate” refers to compounds that further include a stoichiometric or non-stoichiometric amount of a solvent bound by non-covalent intermolecular forces. If the solvent is water, the solvate is referred to as “hydrate.” Pharmaceutically acceptable solvates and hydrates are complexes that, for example, may include from 1 to about 100, or from 1 to about 10, or from one to about 2.3 or 4 molecules of water or a solvent. In some embodiments, the hydrate may be a channel hydrate. It should be understood that the term “compound” in this application covers the compound and solvates of the compound, as well as mixtures thereof.

In some embodiments, the term “hydrate” includes, but is not limited to, hemihydrate, monohydrate, dihydrate, trihydrate and the like.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds described herein may contain one or more asymmetric centers and may thus give rise to diastereomers and optical isomers. The present invention includes all such possible optical isomers, diastereomers as well as their racemic mixtures, their substantially pure resolved enantiomers, all possible geometric isomers, and pharmaceutically acceptable salts thereof. The above Formula (I) is shown without a definitive stereochemistry at certain positions. The present invention includes all stereoisomers of Formula (I) and pharmaceutically acceptable salts thereof. Further, mixtures of stereoisomers as well as isolated specific stereoisomers are also included.

In some embodiments, this invention encompasses the use of various optical isomers of the compound of the invention. It will be appreciated by those skilled in the art that the compounds of the present invention contain at least one chiral center. Accordingly, the compounds used in the methods of the present invention may exist in, and be isolated in, optically-active or racemic forms. Some compounds may also exhibit polymorphism. It is to be understood that the present invention encompasses any racemic, optically-active, polymorphic, or stereoisomeric form, or any combination thereof, which form possesses properties useful in the treatment of androgen-related conditions described herein. In some embodiments, the compounds of the invention are optically pure. In other embodiments, the compounds of the invention are a racemic mixture. It is well known in the art how to prepare optically-active forms (for example, by resolution of the racemic form by recrystallization techniques, by synthesis from optically-active starting materials, by chiral synthesis, or by chromatographic separation using a chiral stationary phase).

During the course of the synthetic procedures used to prepare such compounds, or in using racemization or epimerization procedures known to those skilled in the art, the products of such procedures can be a mixture of stereoisomers.

In some embodiments, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes “pharmaceutically acceptable salts” of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compound of the invention include the conventional non-toxic salts of the parent compound formed, for example, from non-toxic inorganic or organic acids. The pharmaceutically acceptable salts of the compound of the invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. In some embodiments, the solvent is a nonaqueous media like ether, ethyl acetate, ethanol, isopropanol, or acetonitrile.

The pharmaceutically acceptable salts of the compound of the invention can be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of an existing salt for another ion or suitable ion-exchange resin.

Possible pharmaceutically acceptable salts are well known in the art. For example, S. M. Berge, et al. describe pharmaceutically acceptable salts in detail in J. Pharmaceutical Sciences, 66:1-19, 1977; incorporated herein by reference.

Typically, a pharmaceutically acceptable salt form of a compound can be prepared in situ during the final isolation and purification of the compound, or separately by reacting the free base functionality with a suitable organic or inorganic acid.

Suitable acids for preparation of the pharmaceutically acceptable salts include, but are not limited to, inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods used in the art such as ion exchange.

Other pharmaceutically acceptable salts can include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and quaternary ammonium salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The invention further includes derivatives of the compound of the invention. The term “derivatives” includes but is not limited to ether derivatives, acid derivatives, amide derivatives, ester derivatives and the like.

The invention further includes metabolites of the compound of the invention. The term “metabolite” means any substance produced from another substance by metabolism or a metabolic process.

The invention further includes pharmaceutical products of the compound of the invention. The term “pharmaceutical product” means a composition suitable for pharmaceutical use (pharmaceutical composition), as defined herein.

The invention further includes prodrugs of the compound of the invention. The term “prodrug” means a substance which can be converted in vivo into a biologically active agent by such reactions as hydrolysis, esterification, de-esterification, activation, salt formation and the like.

### Cell Therapies and Vaccines

In some embodiments, the hydrogel composition provides a method of cell therapy for a subject in need thereof. In some embodiments, the cell therapy is a vaccine.

A “vaccine” is a biological preparation that can be prophylactic (to prevent or ameliorate the effects of a future infection by a natural or “wild” pathogen), or therapeutic (to fight a disease that has already occurred, such as cancer). A prophylactic vaccine provides active acquired immunity to a particular infectious disease. It typically contains an agent that resembles a disease-causing microorganism and is often made from weakened or killed forms of the microbe, its toxins, or one of its surface proteins. The agent stimulates the body's immune system to recognize the agent as a threat, destroy it, and to further recognize and destroy any of the microorganisms associated with that agent that it may encounter in the future.

In some embodiments, the hydrogel composition comprises a vaccine. In some embodiments, the hydrogel composition comprises a therapeutic vaccine.

### Treatment Methods

Disclosed herein is a method for treating, inhibiting, ameliorating, or alleviating inflammation in a subject in need thereof, comprising administering to the subject a natural polymer suspension composition comprising a pharmaceutical composition comprising a compound or a pharmaceutically acceptable salt thereof.

In one embodiment, the method further comprises administering to the subject a polymer suspension composition disclosed herein.

In some embodiments, the present invention further comprises administering to the subject at least once daily throughout the duration of therapy period: days, weeks, months, or years, with intervals that follow treatment outcome.

The exact dose and regimen of administration of the composition will necessarily be dependent upon the therapeutic or nutritional effect to be achieved and may vary with the particular formula, the route of administration, and the age and condition of the individual subject to whom the composition is to be administered.

In some embodiments, methods of using the hydrogel comprise expansion of the lymphatic system or network. In some embodiments, methods of using the hydrogel comprise expansion of the circulatory system or network. Expansion of the lymphatic system or network and/or expansion of the circulatory system or network provides methods for treating, inhibiting, ameliorating, or alleviating a disease or abnormal physiological condition in a subject in need thereof.

“Inflammation,” as defined used, is part of the complex biological response of body tissues to harmful stimuli, such as pathogens, damaged cells, or irritants and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair. The five classical signs of inflammation are heat, pain, redness, swelling, and loss of function. Inflammation is a generic response, and therefore, a mechanism of innate immunity. Causes of inflammation can be physical (e.g., injury/trauma, burn, frostbite, foreign body, ionizing radiation), biological (e.g., infection, immune reactions due to hypersensitivity, stress), chemical (e.g., chemical irritant, toxin, alcohol), or psychological (e.g., excitement). Inadequate inflammation can lead to tissue damage by the initial stimulus, while a prolonged response is associated with the development of chronic disease or disorder. Inflammation can be “acute” or “chronic.”

“Acute inflammation,” as used herein, is initiated by resident immune cells already present in the involved tissue, mainly resident macrophages, dendritic cells, histiocytes, Kupffer cells and mast cells. These cells possess surface receptors known as pattern recognition receptors (PRRs), which recognize and bind two subclasses of molecules: pathogen-associated molecular patterns (PAMPs) and damage-associated molecular patterns (DAMPs). PAMPs are compounds that are associated with various pathogens, but which are distinguishable from host molecules. DAMPs are compounds that are associated with host-related injury and cell damage. In response to an infection, burn, or other injuries, these cells are activated (i.e., a PRR recognizes a PAMP or DAMP) and release inflammatory mediators responsible for the clinical signs of inflammation. Vasodilation and its resulting increased blood flow cause the redness and increased heat. Increased permeability of the blood vessels results in an exudation (leakage) of plasma proteins and fluid into the tissue (edema), resulting in swelling. Some of the released plasma-derived mediators (e.g., bradykinin) increase the sensitivity to pain. The mediator molecules also alter the blood vessels to permit the migration of leukocytes, mainly neutrophils and macrophages, outside of the blood vessels (extravasation) into the tissue. The neutrophils migrate along a chemotactic gradient created by the local cells to reach the site of injury. The loss of function may be a neurological reflex in response to pain. Plasma-derived mediators include, but are not limited to, C3, C5a, Factor XII, membrane attack complex, plasmin, thrombin. C3 and C5a, for example, stimulate histamine release by mast cells. This phase of inflammation is usually short-lived and is generally maintained by a cellular response. The cellular component involves leukocytes, which normally reside in blood and must move into the inflamed tissue via extravasation to aid in inflammation. Some act as phagocytes, ingesting bacteria, viruses, and cellular debris. Others release enzymatic granules that damage pathogenic invaders. The cellular response is mediated by leukocytes, which also release inflammatory mediators that develop and maintain the inflammatory response. In general, acute inflammation is mediated by granulocytes, whereas chronic inflammation is mediated by mononuclear cells such as monocytes and lymphocytes. Various leukocytes, particularly neutrophils, are critically involved in the initiation and maintenance of inflammation, resulting in a cascade of cytokines, chemokines, ligands, and receptors. Examples of cell-derived mediators include, but are not limited to, enzymes (e.g., lysosome granules, tryptase), monoamines (e.g., histamine), cytokines (e.g., interferon-gamma [IFN-γ], interleukin-1 [IL-1], tumor necrosis factor-alpha [TNF-α]), chemokines (e.g., interleukin-8 [IL-8]), eicosanoids (e.g., leukotriene B4, LTC4, LTD4, 5-oxo-eicosatetraenoic acid, 5-HETE, prostaglandins), and soluble gases (e.g., nitric oxide). Typical outcomes of inflammation include resolution (complete restoration of the inflamed tissue to normal), fibrosis (scarring), abscess formation (pus-filled cavity), or chronic inflammation (e.g., due to persistence of the injurious agent or state, resulting in damage to the body's own tissues by continued macrophage response).

“Chronic inflammation,” as used herein, may result from situations in which the injurious agent persists, leading to a continued inflammatory response. This process, marked by inflammation lasting many days, months or even years, may lead to the formation of a chronic wound, rash, or other chronic inflammatory condition. Chronic inflammation is characterized by the dominating presence of macrophages in the injured tissue. These cells are powerful defensive agents of the body, but the toxins they release (including reactive oxygen species) are injurious to the organism's own tissues as well as invading agents. In addition, other cells involved in the inflammatory response (e.g., mast cells) are often stabilized, continuously engaging in an inflammatory response, such as in the case of allergies, hypersensitivities, asthma, and chronic obstructive pulmonary disease. As a consequence, chronic inflammation is almost always accompanied by tissue destruction and may result in a chronic disease or other medical condition. Examples of diseases or other medical conditions in which chronic inflammation has been implicated, either as cause or effect, include, but are not limited to, allergic and/or hypersensitivity reactions, allergies, Alzheimer's disease, arthritis and other joint diseases, asthma, atherosclerosis, acne vulgaris, autoimmune diseases, autoinflammatory diseases, bronchitis, cardiovascular disease (CVD; including ischemic heart disease), celiac disease, chronic obstructive pulmonary disease (COPD), chronic prostatitis, colitis, cystitis, dermatitis, diabetes, diverticulitis, familial Mediterranean fever, glomerulonephritis, chronic gout, gouty arthritis, hidradenitis suppurativa, inflammatory bowel diseases, interstitial cystitis, lichen planus, mast cell activation syndrome, mastocytosis, otitis, myopathies, pelvic inflammatory disease, chronic peptic ulcer, periodontitis, psoriasis, psoriatic arthritis, reflex sympathetic dystrophy/complex regional pain syndrome (RSD/CRPS), reperfusion injury, rheumatic fever, rheumatoid arthritis (as possibly chronic osteoarthritis), psoriasis, rhinitis, sarcoidosis, transplant rejection, vasculitis, and some cancers. In addition to injury, infections (e.g., bacterial, viral [COVID-19], fungal, parasitic), and other causes of chronic inflammation, obesity, smoking, periodontal disease, peptic ulcer, arthritis, bursitis, tendonitis, phlebitis, tonsilitis, reflex sympathetic dystrophy/complex regional pain syndrome (RSD/CRPS), asthma, tuberculosis, ulcerative colitis, Crohn's disease, sinusitis, hepatitis, rheumatoid arthritis, and high cholesterol (e.g., high levels of low-density lipoprotein [LDL]), poor nutrition, stress, and insomnia have also been implicated as causal agents of chronic inflammation. Intestinal microbiota can fuel metabolic inflammation, e.g., by triggering an influx of bacteria-derived lipopolysaccharides (LPS) into systemic circulation. Alterations in gut microbiota composition are associated with a variety of disease states, including those associated with inflammation, such as obesity, diabetes, and inflammatory bowel disease (IBD). These diseases can have far-reaching effects, leading to increased risks of heart diseases, heart attack, and stroke (including ischemic stroke). It is noteworthy that some causes may also be effects, further contributing to the cycle of damage. Diagnosis of chronic inflammation can include a blood test measuring the amount of C-reactive protein (CRP), which rises in response to inflammation or an erythrocyte sedimentation rate. A CRP level between 1 and 3 milligrams per liter of blood often signals a low, yet chronic, level of inflammation.

Chronic “systemic inflammation” (SI), as used herein, is the result of release of pro-inflammatory cytokines from immune-related cells and the chronic activation of the innate immune system. It can contribute to the development or progression of certain conditions. Release of pro-inflammatory cytokines and activation of the innate immune system may be the result of either external (biological or chemical agents) or internal (genetic mutations/variations) factors, as well as aberrations in the microbiome (e.g., in the intestinal [gut] microbiota). Lack of control by tolerogenic dendritic cells (TDC) and T-regulatory cells (Treg) is a possible primary risk factor for the development of SI.

“Neuroinflammation,” as used herein, is inflammation of the nervous tissue. It may be initiated in response to a variety of cues, including infection, traumatic brain injury, toxic metabolites, or autoimmunity. In the central nervous system (CNS), including the brain and spinal cord, microglia are the resident innate immune cells that are activated in response to these cues. The CNS is typically an immunologically privileged site because peripheral immune cells are generally blocked by the blood-brain barrier (BBB), a specialized structure composed of astrocytes and endothelial cells. However, circulating peripheral immune cells may surpass a compromised or “leaky” BBB and encounter neurons and glial cells expressing major histocompatibility complex molecules, perpetuating the immune response. Although the response is initiated to protect the central nervous system from the infectious agent, the effect may be toxic and widespread inflammation as well as further migration of leukocytes through the blood-brain barrier. Neuroinflammation is usually chronic. While acute inflammation typically follows injury to the central nervous system immediately, and is characterized by inflammatory molecules, endothelial cell activation, platelet deposition, and tissue edema, chronic neuroinflammation is the sustained activation of glial cells and recruitment of other immune cells into the brain. It is chronic neuroinflammation that is typically associated with neurodegenerative diseases. Common causes of chronic neuroinflammation include, but are not limited to, toxic metabolites, autoimmunity, aging, microbes, viruses, traumatic brain injury, spinal cord injury, air pollution, smoking/passive smoke. The neuroimmune response relies primarily on glial cells and cytokines.

“Anti-inflammatory drugs,” as used herein, are pharmaceuticals that reduce inflammation. They include about half of analgesics, remedying pain by reducing inflammation in contrast to opioids, which affect the central nervous system to block pain signaling to the brain. Examples include, but are not limited to non-steroidal anti-inflammatory drugs (NSAID), COX-2 inhibitors, antileukotrienes, ImSAIDs, etc.

“Non-steroidal anti-inflammatory drugs” (NSAID), as used herein, are drugs that alleviate pain by counteracting the cyclooxygenase (COX) enzyme. Inhibition of COX enzymes inhibits prostaglandin synthesis, thereby preventing PGs from inducing inflammation. Examples of NSAIDS include, but are not limited to, aspirin, ibuprofen, naproxen, and other NSAIDS.

“COX-2 inhibitors,” as used herein, are a subset of NSAIDs that specifically target cyclooxygenase-2 (COX-2), an enzyme responsible for inflammation and pain. Examples include, but are not limited to, celecoxib, rofecoxib, etoricoxib.

“Antileukotrienes,” as used herein, are anti-inflammatory agents which function as leukotriene-related enzyme inhibitors (arachidonate 5-lipoxygenase) or leukotriene receptor antagonists (cysteinyl leukotriene receptors) and consequently oppose the function of these inflammatory mediators. Although they are not used for analgesic benefits, they are widely utilized in the treatment of diseases related to inflammation of the lungs such as asthma and COPD, as well as sinus inflammation in allergic rhinitis. Examples include, but are not limited to, leukotriene receptor antagonists (e.g., montelukast, zafirlukast) and leukotriene synthesis inhibitors (e.g., zileuton), such as those blocking 5-lipooxygenase.

“Immune Selective Anti-Inflammatory Derivatives” (ImSAIDs)” as used herein, are a class of peptides which have diverse biological properties, including anti-inflammatory properties. ImSAIDs work by altering the activation and migration of inflammatory cells, which are immune cells responsible for amplifying the inflammatory response. Examples include, but are not limited to, phenylalanine-glutamine-glycine (FEG) and its D-isomeric form (feG).

A “fibroma” is a benign tumor that is composed of fibrous or connective tissue. They can grow in all organs, arising from mesenchyme tissue. The term “fibroblastic” or “fibromatous” is used to describe tumors of the fibrous connective tissue. A “fibrosarcoma” is a malignant tumor (a malignant fibroma).

A “cancer” is one of a group of diseases characterized by uncontrollable growth and having the ability to invade normal tissues and to metastasize to other parts of the body. Cancers have many causes, including, but not limited to, diet, alcohol consumption, tobacco use, environmental toxins, heredity, and viral infections. In most instances, multiple genetic changes are required for the development of a cancer cell. Progression from normal to cancerous cells involves a number of steps to produce typical characteristics of cancer including, e.g., cell growth and division in the absence of normal signals and/or continuous growth and division due to failure to respond to inhibitors thereof; loss of programmed cell death (apoptosis); unlimited numbers of cell divisions (in contrast to a finite number of divisions in normal cells); aberrant promotion of angiogenesis; and invasion of tissue and metastasis.

In some embodiments, methods of using the hydrogel composition comprise treating, inhibiting, ameliorating, or alleviating a cancer or a tumor in a subject or a growth or a metastasis thereof, comprising a step of administering the hydrogel composition to the subject in a subject in need thereof. In some embodiments, methods of using the hydrogel composition comprise treating, inhibiting, ameliorating, or alleviating a cancer or a tumor in a subject in need thereof. In some embodiments, methods of using the hydrogel composition comprise treating, inhibiting, ameliorating, or alleviating metastasis of a cancer in a subject in need thereof. In some embodiments, methods of using the hydrogel composition further comprise increasing survival in the subject. In some embodiments, methods of using the hydrogel composition further comprise reducing or inhibiting metastases infiltration.

A “pre-cancerous” condition, lesion, or tumor is a condition, lesion, or tumor comprising abnormal cells associated with a risk of developing cancer. Non-limiting examples of pre-cancerous lesions include colon polyps (which can progress into colon cancer), cervical dysplasia (which can progress into cervical cancer), and monoclonal monopathy (which can progress into multiple myeloma). Premalignant lesions comprise morphologically atypical tissue which appears abnormal when viewed under the microscope, and which are more likely to progress to cancer than normal tissue.

A “non-cancerous tumor” or “benign tumor” is one in which the cells demonstrate normal growth, but are produced, e.g., more rapidly, giving rise to an “aberrant lump” or “compact mass,” which is typically self-contained and does not invade tissues or metastasize to other parts of the body. Nevertheless, a non-cancerous tumor can have devastating effects based upon its location (e.g., a non-cancerous abdominal tumor that prevents pregnancy or causes a ureter, urethral, or bowel blockage, or a benign brain tumor that is inaccessible to normal surgery and yet damages the brain due to unrelieved pressure as it grows).

Non-limiting examples include esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome. In another related aspect, the tumor or cancer comprises a metastasis of a tumor or cancer. In some embodiments, a solid tumor treated using a method described herein, originated as a blood tumor or diffuse tumor.

In some embodiments, the method of treating, inhibiting, ameliorating, or alleviating a tumor or cancer comprises treatment of solid tumors or cancers. In some embodiments, the method of treating, inhibiting, ameliorating, or alleviating a tumor or cancer comprises treatment of a melanoma, a fibroma, or fibrosarcoma.

The present invention further provides a combination therapy. The term “combination therapy” means the administration of two or more therapeutic agents to treat a therapeutic disorder described in the present invention. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

The terms “subject” and “patient” are used interchangeably herein when referencing, for example, a mammalian or avian subject. The terms “subject” and “patient” are used interchangeably herein when referencing, for example, a mammalian subject, such as a human patient. In some embodiments, the subject in the method of the invention is a higher vertebrate (i.e., a mammal or a bird). In some embodiments, the subject in the method of the invention is a mammal. In some embodiments, the subject in the method of the invention is a human patient.

The term “treatment” or “treating” as used herein refers to the administering of a therapeutic effective amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

Unless defined otherwise, all technical and scientific terms used herein have the same meaning as commonly understood to one of ordinary skill in the art.

Unless otherwise indicated, all numbers expressing quantities, ratios, and numerical properties of ingredients, reaction conditions, and so forth used in the specification and claims are to be understood as being modified in all instances by the term “about”. All parts, percentages, ratios, etc. herein are by weight unless indicated otherwise.

As used herein, the singular forms “a” or “an” or “the” are used interchangeably and intended to include the plural forms as well and fall within each meaning, unless expressly stated otherwise. Also as used herein, “at least one” is intended to mean “one or more” of the listed elements. For example, the term “a compound” or “at least one compound” may include a plurality of compounds, including mixtures thereof. Singular word forms are intended to include plural word forms and are likewise used herein interchangeably where appropriate and fall within each meaning, unless expressly stated otherwise. Except where noted otherwise, capitalized and non-capitalized forms of all terms fall within each meaning.

“Consisting of” shall thus mean excluding more than traces of other elements. The skilled artisan would appreciate that while, in some embodiments the term “comprising” is used, such a term may be replaced by the term “consisting of”, wherein such a replacement would narrow the scope of inclusion of elements not specifically recited. The terms “comprises”, “comprising”, “includes”, “including”, “having” and their conjugates encompass “including but not limited to”.

The term “about” or “approximately” means within an acceptable error range for the particular value as determined by one of ordinary skill in the art, which will depend in part on how the value is measured or determined. In some embodiments, the term “about” refers to a deviance of between 0.0001-5% from the indicated number or range of numbers. In some embodiments, the term “about” refers to a deviance of between 1-10% from the indicated number or range of numbers. In some embodiments, the term “about” refers to a deviance of up to 25% from the indicated number or range of numbers. In some embodiments, the term “about” refers to +10%.

Throughout this application, various embodiments may be presented in a range format. It should be understood that the description in range format is merely for convenience and brevity and should not be construed as an inflexible limitation on the scope of certain embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is to be understood that the phraseology or terminology employed herein, and not otherwise defined, is for the purpose of description only and not of limitation. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. Further, reference to values stated in ranges includes each and every value within that range. Certain features of the disclosed compositions and methods which are described herein in the context of separate aspects may also be provided in combination in a single aspect. Alternatively, various features of the disclosed compositions and methods that are, for brevity, described in the context of a single aspect, may also be provided separately or in any subcombination.

Any patent, patent application publication, or scientific publication, cited herein, is incorporated by reference herein in its entirety.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Methods of Using Pharmaceutical- and/or Agent-Loaded Cell-Based Hydrogel Vaccine to Form a Biomaterial Scaffold In Situ

A polymer (e.g., collagen, gelatin, fibrin, or a collagen-fibrin combination) suspension composition is prepared as described herein. Cells are formulated in the suspension and/or one or more therapeutics/biotherapeutics are loaded in the polymer suspension composition (e.g., at a temperature lower than the body temperature of the subject). A subject is injected with the cell-formulated and/or therapeutic/biotherapeutic-loaded collagen suspension vaccine via subcutaneous injection or intraperitoneal injection, and the hydrogel forms, e.g., in response to the temperature change.

A biomaterial scaffold forms in situ, e.g., encapsulating the therapeutics/biotherapeutic and/or cells. The therapeutics/biotherapeutics and/or cells form, e.g., a lymphoid tissue, a high endothelial venule, or another lymphoid structure. The therapeutics/biotherapeutics and/or cells interact with the subject's immune system to attack, directly or indirectly, tumor or cancer cells.

### Example 2: Methods of Using Biotherapeutic-Loaded Hydrogels as Anti-Cancer Vaccines

The hydrogels disclosed herein are loaded with a variety of therapeutics/biotherapeutics for multiplex anti-tumor or anti-cancer vaccines.

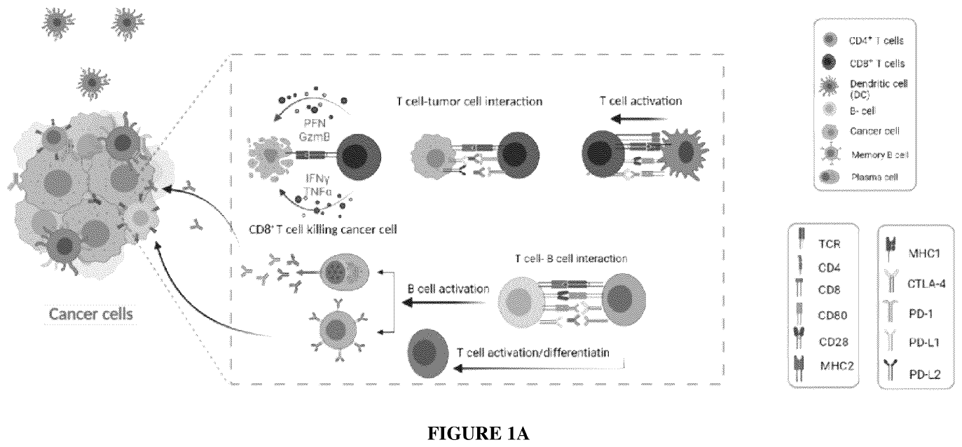

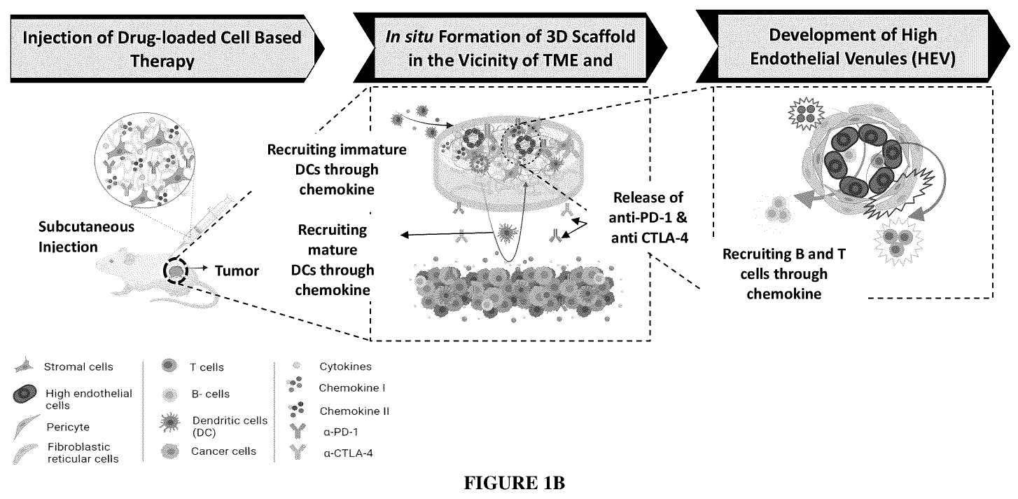

FIG. 1A is a schematic depicting exemplary immune interactions for the development of approaches for methods of using a therapeutic/biotherapeutic-loaded cell-based hydrogel vaccine for the treatment of cancer cells in vivo. A dendritic cell (DC) with cluster of differentiation 80 (CD80) and major histocompatibility class 1 (MHC1) activates a cluster of differentiation 8+ (CD8+) cytotoxic T-cell having major histocompatibility class 2 (MHC2), T-cell receptor (TCR), cluster of differentiation 28 (CD28), and cytotoxic T-lymphocyte-associated protein 4 (CTLA-4) antibody (top right). A T-cell with programmed cell death protein 1 (PD-1) antibodies and T-cell receptors (TCR) interacts with a tumor cell having major histocompatibility complex 2 (MHC2), programmed death ligand 1 (PD-L1) antibody, and programmed death ligand 2 (PD-L2) antibody (top center). The TCR on the CD8+ cytotoxic T-cell recognizes the major histocompatibility complex 1 (MHC1) on the cancer cell (top left), and the CD8+ cytotoxic T-cell secretes interferon-gamma (IFN-gamma, IFN-γ), tumor necrosis factor-alpha (TNF-alpha, TNF-α), granzyme B (GzmB, GrB), and the pore-forming protein perforin (PFN). PFN facilitates PFN facilitates the entry of granzymes into cells. The CD8+ cytotoxic T-cell kills the cancer cell (top left). A cluster of differentiation 4+ (CD4+) helper T-cell with TCR, CD80, CTLA-4 antibody, and PD-1 antibody interacts with a B-cell having MHC2, CD28, CD80, and PD-L1 antibody (bottom right), resulting in T-cell activation/differentiation (bottom below) and B-cell activation (bottom center) to yield memory B-cells and plasma cells (bottom left). Therefore, cancer cells are attacked overall by cytotoxic CD8+ T-cells, helper CD4+ T-cells, memory B-cells, dendritic cells, and antibodies from plasma cells (left).

One or more therapeutics/biotherapeutics is used to promote the interactions shown here or to recruit and/or facilitate maturation of the immune cells as described herein (e.g., FIG. 1B).

### Example 3: Method of Using Pharmaceutical- and/or Agent-Loaded Cell-Based Hydrogel Vaccine to Form a Biomaterial Scaffold In Situ and to Develop High Endothelial Venules

As depicted in FIG. 1B, a polymer (e.g., collagen) suspension is prepared as described herein. Cells are formulated in the polymer suspension, and biotherapeutics (cytokines, chemokine I, chemokine II, anti-PD-1 antibody, and anti-CTLA-4 antibody) are loaded in the polymer suspension (e.g., at a temperature lower than the body temperature of the subject). A mammal with a tumor is injected with the cell-formulated, biotherapeutic-loaded polymer suspension via subcutaneous injection, and the hydrogel forms, e.g., in response to the temperature change, thereby encapsulating the cells and biotherapeutics. FIG. 1B is a schematic depicting an exemplary method of using a pharmaceutical- and/or agent-loaded cell-based hydrogel vaccine for the treatment of a tumor in vivo. FIG. 1B demonstrates subcutaneous injection into a subject of a suspension comprising polymer, cells, cytokines, chemokines (chemokine I and chemokine II), and antibodies (anti-programmed cell death-1 antibody [anti-PD-1/α-PD-1 antibody] and anti-cytotoxic T-lymphocyte-associated protein 4 [anti-CTLA-4/α-CTLA-4 antibody]) (left).

As shown in FIG. 1B, a biomaterial scaffold forms in situ from the immune checkpoint blockade antibody-releasing hydrogel (top center). The biotherapeutics interact with the subject's immune system to attack, directly or indirectly, tumor or cancer cells. The chemokines in the scaffold recruit immature dendritic cells (DC). The scaffold releases anti-PD-1 antibodies and anti-CTLA-4 antibodies, and mature DC are also recruited through chemokines to attack the tumor. High endothelial venules (HEV) are formed in the biomaterial scaffold (right), and chemokines from the scaffold recruit B-cells and T-cells via the HEV, where they can interact with antigen presenting cells (APC). Ultimately, the biotherapeutics interact with the subject's immune system to attack, directly or indirectly, tumor or cancer cells. FIG. 1B depicts subcutaneous in situ-formation of the biomaterial scaffold (top center) in peripheral tissue, allowing recruitment of immature and mature dendritic cells (DCs) by the chemokines and release of the anti-PD-1 and anti-CTLA-4 antibodies while the cells growth and proliferate to expand the lymphoid network by developing high endothelial venules (HEV) where the chemokines recruit B-cells and T-cells (right). The anti-PD-1 and anti-CTLA-4 antibodies, dendritic cells, B-cells, and T-cells target the tumor cells (bottom center).

### Example 4: Preparation and Testing of Hydrogel Compounds

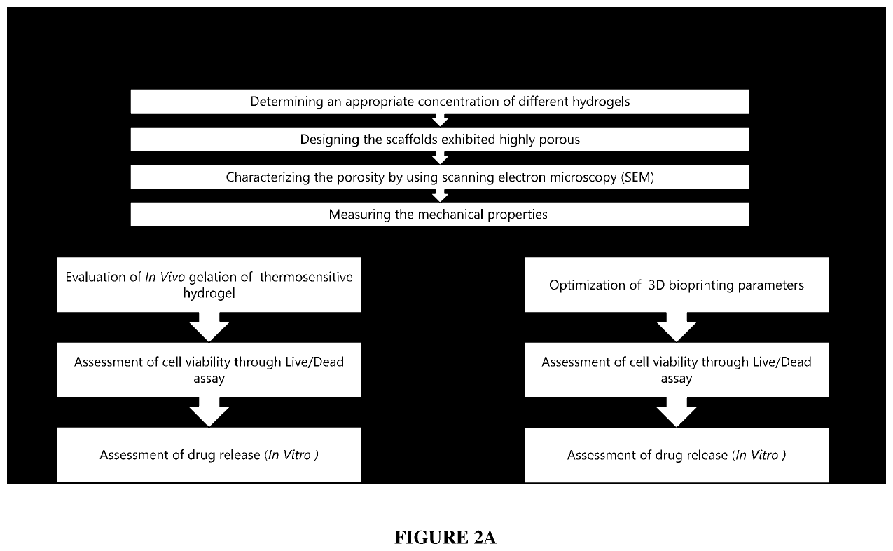

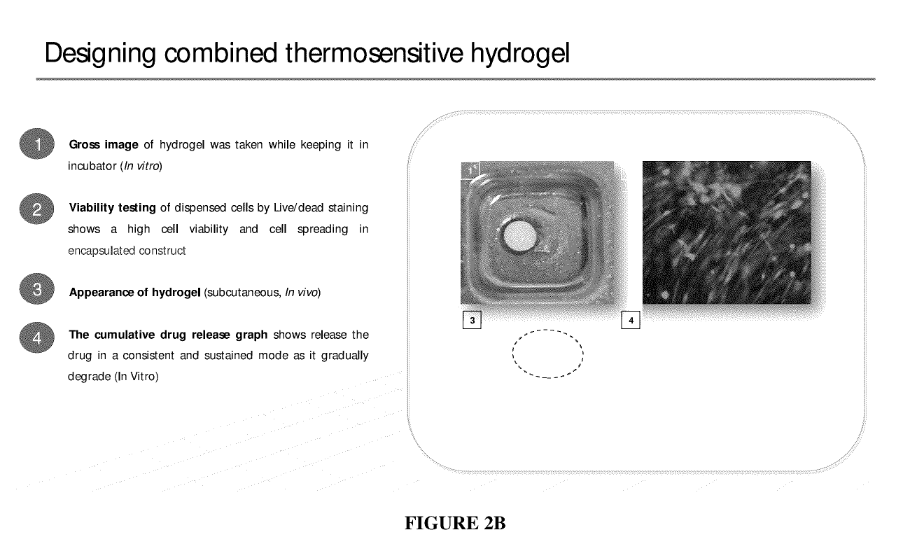

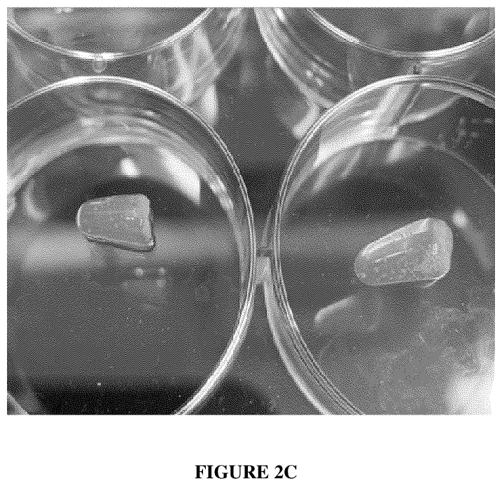

Disclosed herein are processes for designing hydrogel compounds of the present invention. An exemplary scheme for designing and testing hydrogel compounds is shown in the flowchart depicted in FIGS. 2A-2B.

Various concentrations of hydrogel (or of hydrogels of different compositions) are prepared and made to test composition and/or concentration. The scaffolds are designed to be highly porous. The porosity of the various hydrogels is then characterized, e.g., by using scanning electron microscopy (SEM), and the mechanical properties are measured.

Evaluation of the in vivo gelation of thermosensitive hydrogels is conducted. Additionally, three-dimensional (3D) bioprinting parameters are optimized.

If the hydrogels comprise cells (e.g., stromal cells, adipose cells, stem cells, etc.), the hydrogels are assessed for cell viability through, e.g., a Live/Dead assay. If the hydrogels comprise, e.g., drugs and other pharmaceutical compositions, cytokines, chemokines, nucleic acids, antibodies or antigen binding domains, other proteins, and/or other components, the hydrogels are also assessed for release (in vitro or in vivo) of, e.g., drugs and other pharmaceutical compositions, cytokines, chemokines, nucleic acids, antibodies or antigen binding domains, other proteins, and/or other components.

### Example 5: Observation and Testing of Hydrogels

As shown in FIG. 2A, the hydrogels prepared are subjected to various observations and testing.

FIG. 2B shows images of an exemplary hydrogel and testing thereof: (1) a gross photographic image of the hydrogel in vitro in an incubator at Day 3 (panel 1, upper left); electron micrograph of results of viability testing of dispersed cells by Live/Dead staining (Live=green; Dead=red) showing a high cell viability and cell spreading in a hydrogel construct in which the cells were formulated (encapsulated) (panel 2, upper right); photograph of appearance of the hydrogel following subcutaneous implanting in vivo (panel 3, lower left); and a cumulative drug release graph demonstrating release of the drug in a consistent and sustained mode as it gradually degrades in vitro (panel 4, lower right). FIG. 2C is a photograph of gels comprising drug formulation with 3 mg/mL (left) and after drug formulation with 4.5 mg/ml (right).

### Example 6: Preparation of Hydrogels

Several hydrogel preparations were prepared and tested to observe hydrogel properties for eventual cell encapsulation for purposes of hydrogel vaccines. The following protocol was used:
Purpose: Testing collagen gelation at 1 mg/mL, 2 mg/mL, and 3 mg/mL concentrations
Materials:
Collagen (6 mg/mL) (HUMABIOLOGICS™)
0.1N NaOH—to neutralize collagen to pH 7.0-7.5
10× phosphate buffered saline (PBS)—to bring to physiological salt concentrations
Deionized water (DI water, DI H2O, DI H₂O) (produced in-house)
An ice bucket with ice
pH metering paper or pH meter—to monitor collagen pH after adjustment by NaOH 0.1N
Design:
All above materials except pH monitoring paper or meter were placed into an ice bucket with ice.
For a 1 mL collagen suspension:
1. 100 microliters (uL, μL) 10×PBS was added into a microcentrifuge tube, then collagen, a first moiety of DI water as in Table 1 to make a total of 800 uL suspension in a BSC.
2. NaOH 0.1N was added as shown in Table 1 into each collagen tube. The collagen was mixed by pipetting up and down and the pH was checked using the pH paper. (Alternatively, a pH monitor could be used.)
3. Then a second moiety of DI water was added to make up 1 ml suspension total (see Table 1) and mixed by pipetting up and down, while maintaining the collagen in the ice bucket.
4. 200 μL of collagen suspension was transferred into each fresh microcentrifuge tube and placed in the 37C (37° C.) water bath. Gelation was monitored after 30 minutes (min) and one hour (h).

The above materials and methods were used to prepare the hydrogels shown in Table 1.

TABLE 1
Composition of hydrogels for testing.

| | | | | First | NaOH | Second |
|---|---|---|---|---|---|---|
| | Concentration | 10X PBS | Collagen | DI H2O | 0.1N | DI H2O |
| Sample | (mg/mL) | (uL, uL) | (uL, uL) | (uL, uL) | (uL, uL) | (uL, uL) |
| 1 | 1 | 100 | 170 | 530 | 15 | 185 |
| 2 | 2 | 100 | 340 | 360 | 30 | 170 |
| 3 | 3 | 100 | 510 | 190 | 60 | 140 |
| 4 | 1 | 100 | 170 | 530 | 18 | 185 |
| 5 | 2 | 100 | 340 | 360 | 36 | 170 |

### Example 7: Mechanical Testing of Hydrogels

The hydrogels of Example 6 (see Table 1) underwent mechanical testing as follows:
Purpose: Testing of the hydrogels of Example 6 to compare their mechanical properties.
Test results:
The hydrogels were mechanically strong with the following order of strength (strongest to least strong): hydrogel 3, hydrogel 5, hydrogel 2, hydrogel 4, and hydrogel 1 (i.e., 3>5>2>4>1).
Hydrogel 3 was the strongest gel, due to having the highest collagen concentration.
Hydrogel 5 was stronger than hydrogel 2 and hydrogel 4 was stronger than hydrogel 1, due to a slightly higher pH because more NaOH was used.
Hydrogels 3 and 5 were used in the subsequent study.
FRC encapsulation demonstrated excellent cell viability (shown below).

### Example 8: Preparation and Testing of Higher Concentration Hydrogels

The following protocol was used to determine whether a collagen hydrogel could be made with a higher concentration than 4.8 mg/mL:
Purpose: Testing whether a collagen hydrogel can be made with higher concentrations of collagen than 4.8 mg/mL.
Materials:
Hydrochloric acid (FISHER SCIENTIFIC™, 37%, certified American Chemical Society Plus [ACS Plus], cat #A144-500, density 1.19 kg/L) (Note: Hydrochloric acid [FISHER SCIENTIFIC™, 37%, certified ACS Plus, cat #A144-500, density 1.19 kg/L] is the HCl that HUMABIOLOGICS™ uses to dissolve collagen. HUMABIOLOGICS™ uses 0.01M HCl to dissolve collagen at 3 and 6 mg/ml concentrations. However, a higher concentration was used below, as noted.)
Collagen type I lyophilized (HUMABIOLOGICS™)
DI water (produced in house)
Calculations:
1. To make 14 mg/mL collagen, 0.015M HCl was used instead of 0.01M, thereby modifying HUMABIOLOGICS™ 's procedure.
2. 37% HCl is 12M: (37%×1.19 kg/L)=0.44 kg HCl/L. Therefore, 0.44 (kg/L)/36.5 g/mol=12 mol/L (M).
3. 12 M diluted to 1.5 M to 10 mL: 1.5 M×10 ml/12 M=1.25 ml. Therefore, 1.25 mL HCl 12M and 8.75 mL DI water were needed.
4. Then 1.5M was diluted to 0.015M (50 mL): 0.015 M×50 mL/1.5M=0.5 mL. Therefore 49.5 mL DI water and 0.5 mL HCl 1.5M were needed to yield 50 mL 0.015 M HCl.
Method:
1. 100 mg collagen was added to a 50 mL conical tube.
2. 7.14 ml HCl 0.015M was then added to the 50 ml conical tube.
3. The conical tube was shaken at 400 rpm at room temperature (RT) for three h. The conical tube was then incubated in ice-centrifuged at 1200 rpm at 4C (4° C.) for 6 min and another 6 min to remove bubbles.
4. The 14 mg/mL collagen gelation test was performed.
5. The 12 mg/mL collagen gelation test was performed.
6. The 10 mg/mL collagen gelation test was performed.
7. The 9.8 mg/mL collagen gelation test was performed as follows:
   a. Collagen/10×PBS/NaOH/water=140 uL/20 uL/25 uL/15 uL=200 uL (pH is about 7.4-7.6)
   b. Collagen/10×PBS/NaOH/water=140 uL/20 uL/20 uL/20 uL=200 uL (pH is about 7.0-7.2)
   c. Each 9.8 mg/mL formulation was incubated in a water bath at 37C (37° C.) for 30 min. Each formulation formed a good gel.
8. The 6 mg/mL collagen gelation test was performed as follows:
   a. Collagen/10PBS/NaOH/water=85.7 uL/20 uL/20 uL/74 uL=200 uL (pH is about 7.4-7.6)
   b. The 6 mg/mL formulation was incubated in a water bath at 37C (37° C.) for 10 min. It formed a strong gel.

14 mg/mL collagen is very dense. One needs to cut the tip a bit for an easy pipette.

Both 6 and 10 mg/mL formed strong gels in a water bath after 10 min incubation.

The encapsulated cells were viable in 0.015M HCl, as they were in 0.01 M (see FIG. 2C).

### Example 9: Proliferation and Spread of Human Embryonic Kidney (HEK293) Cells in Collagen Type I Hydrogel

Collagen type I hydrogel compositions were prepared at concentrations of 3 mg/mL, 4 mg/mL, and 4.5 mg/mL were prepared. Human embryonic kidney 293 (HEK 293) cells (8M/mL cell density) were added to the collagen type I suspension prior to gelation. On Day 0 and Day 12, the HEK293 cells were stained with LiveDead staining, namely, viable cells stained green (calcein [fluorexon, fluorescein complex]), whereas dead cells stained red (ethidium homodimer-1).

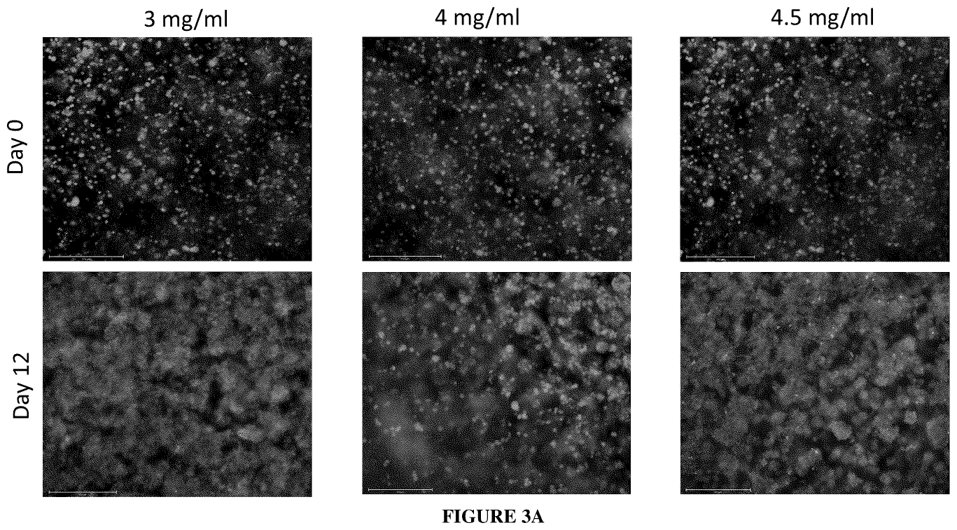

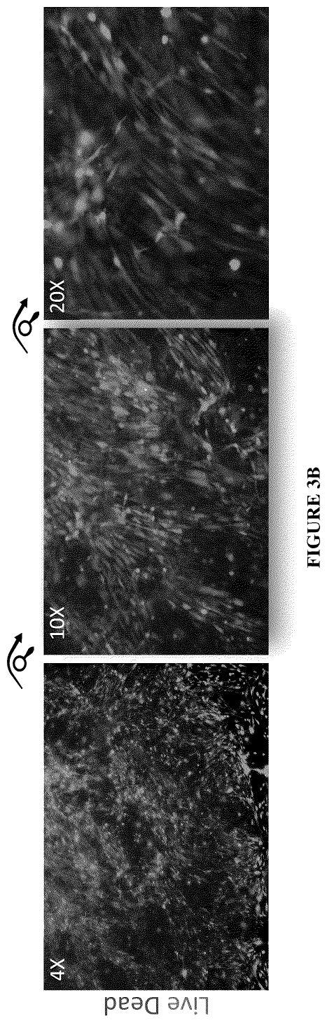

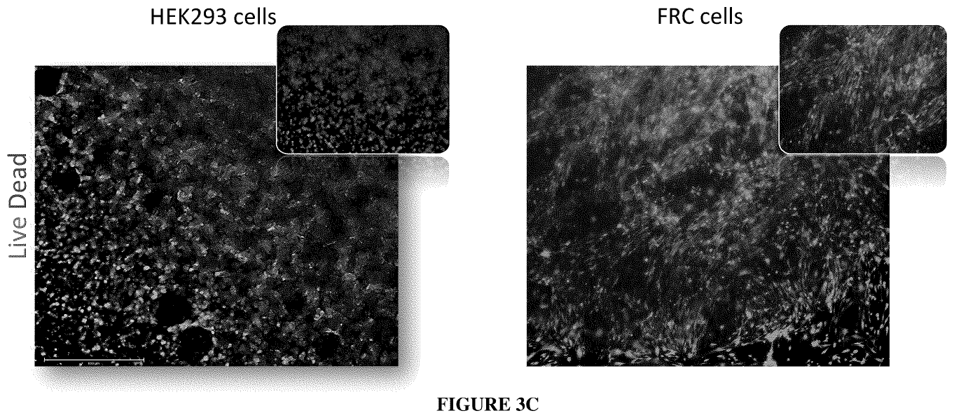

FIG. 3A is a series of fluorescent images depicting the proliferation and spread of HEK293 cells encapsulated into different concentrations of collagen type I (3 mg/mL [left], 4 mg/mL [center], 4.5 mg/mL [right]) on Day 0 (top) and Day 12 (bottom).

### Example 10: Proliferation and Spread of Lymph Cells in Collagen Type I Hydrogel

A collagen type I hydrogel composition was prepared at a concentration 7.4 mg/mL was prepared. Fibroblast reticular cells (FRC) (8M/mL cell density) were added to the collagen type I suspension prior to gelation. On Day 7, the FRC were stained with LiveDead staining, namely, viable cells stained green (calcein [fluorexon, fluorescein complex]), whereas dead cells stained red (ethidium homodimer-1).

FIG. 3B is a series of fluorescent images depicting the proliferation and spread of FRC encapsulated into collagen type I (7.4 mg/mL) with the images taken on day 7 (magnified at 4× [left], 10× [center], and 20× [right]).

### Example 11: Comparison of Proliferation and Spread of HEK293 and Lymph Cells in Collagen Type I Hydrogel

A collagen type I hydrogel composition was prepared at a concentration 7.4 mg/mL was prepared. Either HEK293 cells (8M/mL cell density) or FRC (8M/mL cell density) were added to separate aliquots of collagen type I suspension prior to gelation. On Day 7, the HEK293 cells and the FRC were stained with LiveDead staining, namely, viable cells stained green (calcein [fluorexon, fluorescein complex]), whereas dead cells stained red (ethidium homodimer-1).

FIG. 3C is a comparison of fluorescent images depicting the proliferation and spread of HEK293 cells (left) and FRC (right) (both initially 8M/mL) encapsulated into collagen type I (7.4 mg/mL) with the images taken on day 7 (magnified at 4×\ with insets (upper right of each) magnified at 10×).

### Example 12: Sustained Release of a Model Drug from Collagen Type I Hydrogel

Mouse anti-programmed death ligand 1 antibody (anti-PD-L1 antibody) (BIOXCELL™ cat. no. BE0101; https:/bxcell.com/product/m-pdl-1/) has a molecular weight (Mw) of 150 kilodaltons (kDa). For purposes of this in vitro sustained release study, fluorescein isothiocyanate (FITC) fluorescently-tagged mouse immunoglobulin G (IgG) (INVITROGEN™ cat. no. 31505), which also has a molecular weight of 150 kDa, was substituted.

### Method:

1. Prepared neutralized 4.5 mg/mL collagen type I as follows:
   a. Added 100 ul 10× cold PBS and then 95 ul NaOH 0.1 N into a 15 ml conical tube.
   b. Pipetted 810 ul collagen type I suspension (6 mg/mL) (HUMABIOLOGICS™) to the above tube, pipetted up and down about 10 times.
   c. Checked pH with a pH 5.5-8.0 pH paper to make sure the collagen suspension had a pH of 7.2-7.6.
2. Mixed 200 ug IgG with 400 ul collagen type I suspension (50 ug IgG/100 uL gel). The same suspensions without IgG were prepared to use as controls.
3. Added 100 ul to a 1.5 ml microcentrifuge tube. Incubated at 37C (37° C.) for 30 min for gelation, then added a warm (37C, 37° C.) 1×PBS release medium for the release study.
4. The release was carried out at 37C (37° C.) in a water bath in the dark to avoid photobleaching of the FITC tag.
5. At each predetermined timepoint, the 1×PBS release medium in each tube was removed and another fresh 1 ml PBS was added to the tube.
6. The predetermined timepoints were 3 h, 6 h, 12 h, 1 d, 2 d, 3 d, 5 d, 7 d, 10 d, 14 d, 21 d, 28 d (h=hour(s); d=day(s)).
7. The in vitro sustained release study measured release of FITC-tagged mouse IgG from collagen type I (4.5 mg/mL, pH 7.2-7.6) into 1× phosphate buffered saline (PBS) release medium, as measured by flow cytometry.

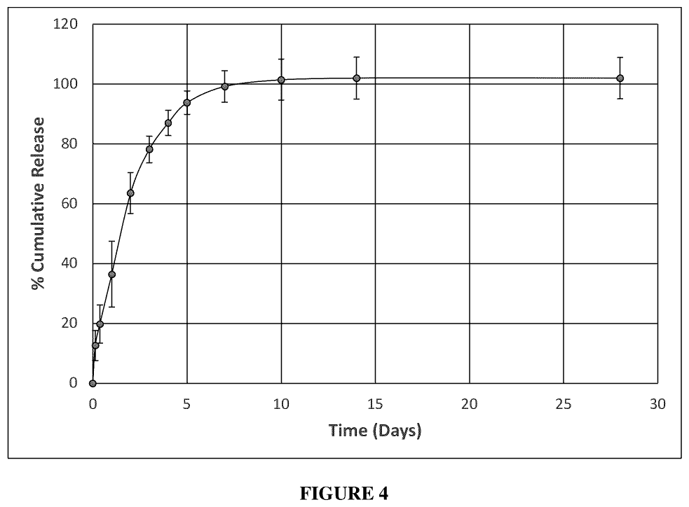

FIG. 4 shows the percentage of fluorescently tagged IgG release as a function of time.

### Example 13: Preparation and Comparison of Proliferation and Spread of HEK293 Cells in Printed Gelatin Methacryloyl (GelMA)/Gelatin Combination Hydrogels of Various

### Concentrations

Several hydrogel preparations were prepared and tested to observe 3D printed hydrogel properties for eventual cell encapsulation for purposes of hydrogel vaccines. The following protocol was used to prepare combination gelatin methacryloyl (GelMA) and gelatin hydrogels of four different concentrations:

### Preparation:

1. With respect to the following, the gelatin methacrylate (lyophilized) (GelMA) (ALLEVI™ cat. no. https://www.allevi3 d.com/product/gelatin-methacrylate-lyophilized/) and gelatin (ALLEVI™ cat. no. GEL; https://www.allevi3 d.com/product/gelatin/) were weighed as shown for each group and placed into a 15-ml conical tube, followed by addition of 750 uL 1× PBS, vortexing, and incubating at 60C:
   a. Group 1: 8% GELMA+2% gelatin (120 mg GELMA+30 mg gelatin)
   b. Group 2: 8% GELMA+4% gelatin (120 mg GELMA+60 mg gelatin)
   c. Group 3: 9% GELMA+3% gelatin (135 mg GELMA+45 mg gelatin)
   d. Group 4: 9% GELMA+4% gelatin (135 mg GELMA+60 mg gelatin)
2. 1% lithium phenyl-2,4,6-trimethylbenzoylphosphinate (LAP) photoinitiator solution was prepared as follows: Weighed 50 mg LAP (ALLEVI™ cat. no. LAP; https://www.allevi3 d.com/product/lap-photoinitiator/), add 5 ml PBS, vortexed until complete dissolution.
3. 750 ul 1% LAP solution was added to each 750 ul GelMA+gelatin tube wrapped in aluminum foil right, vortex. Note: mixed LAP with each group only when that group was ready to be mixed with cells.
4. In a BSC, the bioink was filtered into a 15 ml conical tube wrapped with an aluminum foil using a 3 ml syringe with a 0.2 μm filter in the dark.
5. In an BSC, 5 million HEK293 cells were mixed with 1 ml bioink (8M/mL cell density) in the dark.
6. In a BSC, cell-bioink mixture was loaded into a printing syringe with a 27G (0.21 mm in inner diameter) tip with ¼ inch length. Bubbles in the syringe were removed.
7. The syringe was placed in a large petri dish, which was then placed on ice for 15 min.
8. The syringe was then loaded into the printing nozzle set at 22C for 30 min.
9. Printing was initiated.

On Day 0 and Day 12, the HEK293 cells were stained with LiveDead staining, namely, viable cells stained green (calcein [fluorexon, fluorescein complex]), whereas dead cells stained red (ethidium homodimer-1).

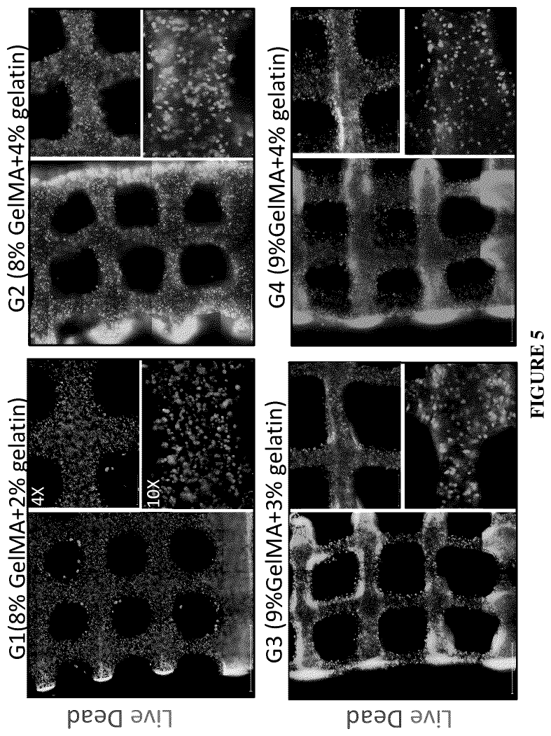

FIG. 5 is a series of groups of fluorescent images depicting proliferation and spread of HEK293 cells (initially 8M/mL) in different concentrations of gelatin methacryloyl (GelMA)+gelatin hydrogel compositions on Day 3 after three-dimensional (3D) printing, in order to compare printing compositions with respect to cell growth in vitro as detected with LiveDead staining, namely, viable cells stained green (calcein [fluorexon, fluorescein complex]), whereas dead cells stained red (ethidium homodimer-1), as shown: G1 (8% GelMA+2% gelatin) (upper left quadrant); G2 (8% GelMA+4% gelatin) (upper right quadrant); G3 (9% GelMA+3% gelatin) (lower left quadrant); and G4 (9% GelMA+4% gelatin) (lower right quadrant). In each quadrant group of fluorescent images, the larger photo (left of each quadrant) is at 10× magnification, while the smaller photos are at 4× magnification (upper right of each quadrant) and 10× magnification (lower right of each quadrant). The greatest cell viability was G2, followed by G1, G4, and G3, in order.

### Example 14: Preparation and Comparison of Proliferation and Spread of Lymph Cells in Printed Gelatin Methacryloyl (GelMA)/Gelatin Combination Hydrogels of Various Concentrations

GelMA/gelatin suspensions were prepared having the following concentrations:
1. 9% GelMA+2% gelatin
2. 8% GelMA
3. 8% GelMA+2% gelatin
4. 8% GelMA+4% gelatin

Prior to gelation, FRC were added at a cell density of 5M/mL and then incubated. After FRC encapsulation in GelMA/gelatin samples maintained in an incubator, the gelatin was dissolved, leaving behind the porous scaffold. Cell growth was detected with LiveDead staining, namely, viable cells stained green (calcein [fluorexon, fluorescein complex]), whereas dead cells stained red (ethidium homodimer-1).

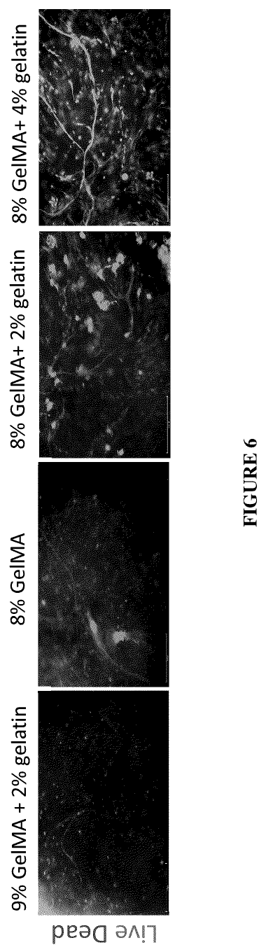

FIG. 6 is a series of fluorescent images (4× magnification) depicting the proliferation and spread of FRC in vitro on Day 7 after encapsulation in hydrogel compositions having different concentrations of GelMA and gelatin, as shown (left to right): 9% GelMA+2% gelatin; 8% GelMA only; 8% GelMA+2% gelatin; and 8% GelMA+4% gelatin. The scaffolds exhibited highly porous and had better cell adhesion and growth than the neat GelMA scaffold. The greatest cell viability was G3, followed by G2 and G1, in order.

### Example 15: Preparation and Comparison of Proliferation and Spread of Cells in Printed Gelatin Methacryloyl (GelMA)/Gelatin Combination Hydrogels of Various Concentrations

GelMA/gelatin suspensions were prepared having the following concentrations:
1. 8% GelMA+4% gelatin.
2. 7% GelMA+3% gelatin.

Prior to gelation, cells were added at a cell density of 8M/mL and then incubated. After cell encapsulation in GelMA/gelatin samples maintained in an incubator, the gelatin was dissolved, leaving behind the porous scaffold. Cell growth was detected with LiveDead staining, namely, viable cells stained green (calcein [fluorexon, fluorescein complex]), whereas dead cells stained red (ethidium homodimer-1).

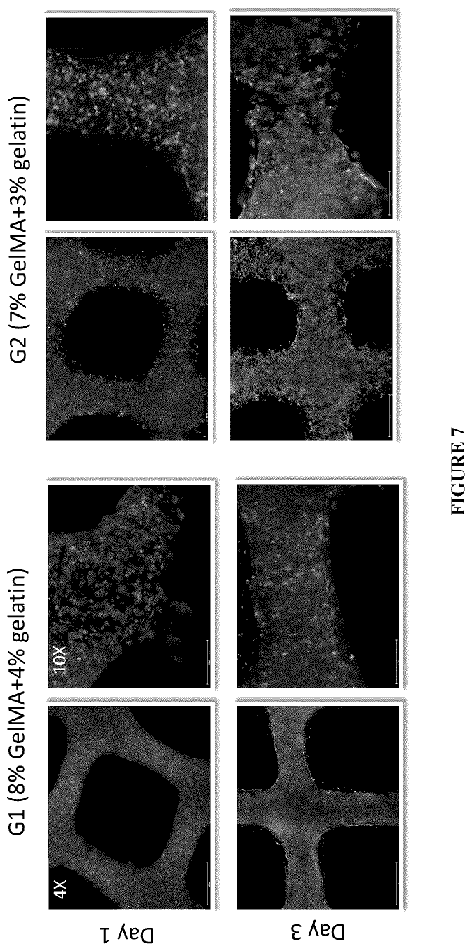

FIG. 7 is a series of fluorescent images depicting proliferation and spread of FRC cells (initial density 8M/mL) encapsulated in hydrogel compositions having different concentrations of GelMA+gelatin following 3D printing. Images were taken of G1 (8% GelMA+4% gelatin) (group of four images on left) and G2 (7% GelMA+3% gelatin) (group of four images on right) at 4× magnification (upper and lower left of each group) and at 10× magnification (upper and lower right of each group) on Day 1 (top row in each group) and Day 3 (bottom row in each group). G2 displayed greater cell viability than G1.

### Example 16: Proliferation and Spread of Lymph Cells in GelMa/Gelatin Combination Hydrogels

In other experiments, 8% GelMA)+4% gelatin combination hydrogels were prepared as described in Example 13 and tested for lymph cell expansion.

The following are examples of lymph cell expansions and cell viability that were conducted:

The predominant form of lymphotoxin-alpha and lymphotoxin-beta on the lymphocyte surface is the lymphotoxin-alpha 1/beta 2 complex (LT-alpha1-beta2, LT-α1β2, LT-α1β2) (e.g., 1 molecule alpha/2 molecules beta), and this complex is the primary ligand for the LT-beta receptor. LT-beta is an inducer of the inflammatory response system and involved in normal development of lymphoid tissue. LT-alpha1-beta2 can interact with receptors such as LT-beta receptors.

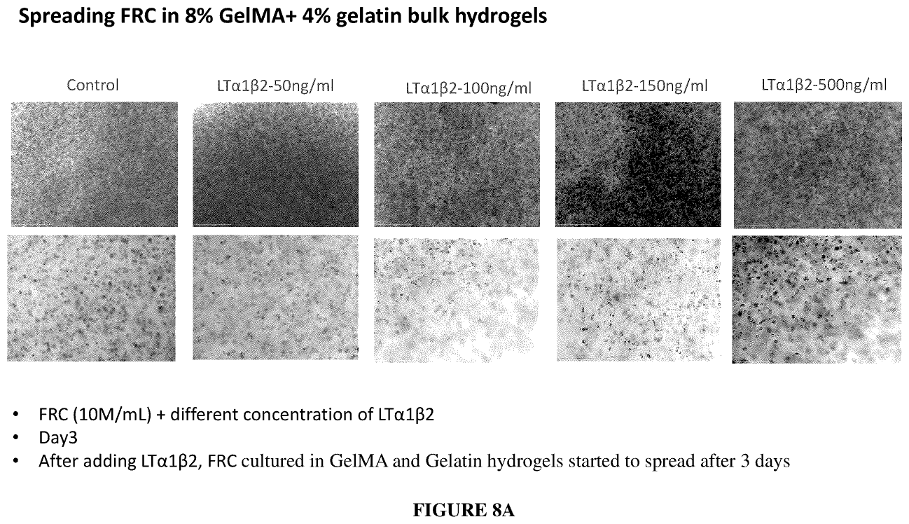

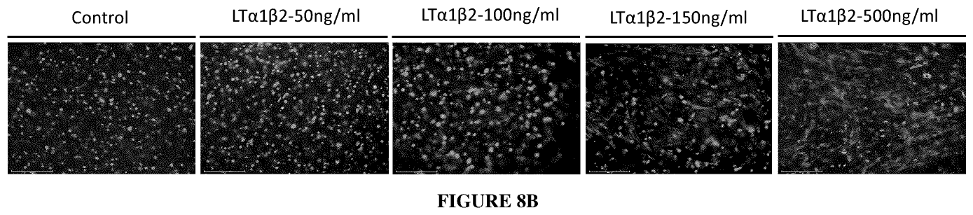

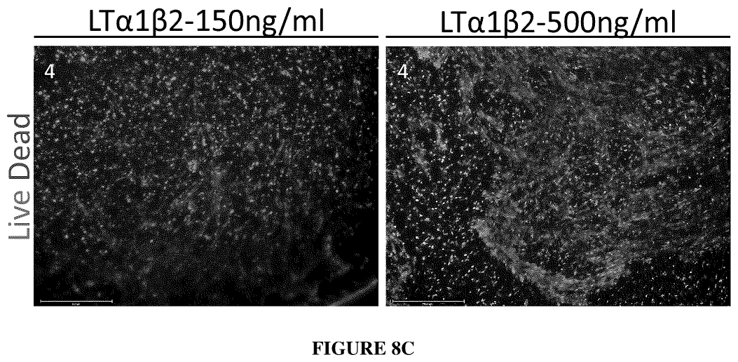

8% GelMA/4% gelatin bulk combination hydrogels were prepared. Fibroblast reticular cells (FRC) were added to an initial concentration of 8M/mL, either without LT-alpha1-beta2 (control) or with different concentrations (50 ng/ml, 100 ng/mL, 150 ng/mL, or 500 ng/ml) of LT-alpha1-beta2. After incubation for 3 days following addition of LT-alpha1-beta2, spreading of the FRC in the hydrogels was observed via bright field microscope images, as shown in FIG. 8A. The top row of images shows 4× magnification. The bottom row of images shows 10× magnification.

These results demonstrate that 8% GelMA/4% gelatin bulk hydrogel, with addition of the primary ligand for the LT-beta receptor, can be used for culturing lymph cells and potentially for developing lymphoid structures with the cultured lymph cells.

### Example 17: Lymph Cell Viability in GelMA/Gelatin Combination Hydrogels

8% GelMA/4% gelatin bulk combination hydrogels were prepared as described in Example 13. Fibroblast reticular cells (FRC) were added to an initial concentration of 8M/mL, either without LT-alpha1-beta2 (control) or with different concentrations (50 ng/ml, 100 ng/ml, 150 ng/ml, or 500 ng/ml) of LT-alpha1-beta2.

Cell growth was detected with LiveDead staining, namely, viable cells stained green (calcein [fluorexon, fluorescein complex]), whereas dead cells stained red (ethidium homodimer-1) on Day 7.

FIGS. 8B-8C are fluorescent images depicting the spread of FRC in vitro on Day 7 after the addition of different concentrations of LT-alpha1-beta2 in 8% GelMA+4% gelatin bulk hydrogels having an initial density of 8M/mL FRC. FIG. 8B shows the proliferation and spread of FRC on Day 7 using the following concentrations of LT-alpha1-beta2 (left to right): Control (no LT-alpha1-beta2), 50 ng/mL LT-alpha1-beta2, 100 ng/mL LT-alpha1-beta2, 150 ng/mL LT-alpha1-beta2, 500 ng/mL LT-alpha1-beta2. Results demonstrated that FRCs spread and formed a great network at 500 ng/ml LT-alpha1-beta2. FIG. 8C shows electron micrographs (4×) depicting the spread of FRC on Day 7 after the addition of different concentrations of LT-alpha1-beta2 in 8% GelMA+4% gelatin bulk hydrogels (left to right): 150 ng/mL LT-alpha1-beta2, 500 ng/mL LT-alpha1-beta2. Results demonstrated that LT-alpha1-beta2 induced FRCs spreading and network formation.

On Day 7 after addition of LT-alpha1-beta2, the cells were stained with Live/Dead staining, and spreading and viability of the FRC in the hydrogels was observed via fluorescence microscope, as shown in FIG. 8B.

It was observed that there was spreading of FRC in 8% GelMA/4% gelatin bulk hydrogel, which was increased by adding 150 ng/mL LT-alpha1-beta2 or 500 ng/mL LT-alpha1-beta2, as shown in FIG. 8C.

These results demonstrate that 8% GelMA/4% gelatin bulk hydrogel, with addition of the primary ligand for the LT-beta receptor, can be used for culturing lymph cells and potentially for developing lymphoid structures with the cultured lymph cells, along with incorporating the primary ligand for the LT-beta receptor, which is an inducer of the inflammatory response system and is involved in normal development of lymphoid tissue.

### Example 18: Sustained Release of a Model Drug from GelMA/Gelatin Combination Hydrogels of Various Concentrations

Mouse anti-programmed death ligand 1 antibody (anti-PD-L1 antibody) (BIOXCELL™ cat. no. BE0101; https:/bxcell.com/product/m-pdl-1/) has a molecular weight (Mw) of 150 kilodaltons (kDa). For purposes of this in vitro sustained release study, fluorescein isothiocyanate (FITC) fluorescently-tagged mouse immunoglobulin G (IgG) (INVITROGEN™ cat. no. 31505), which also has a molecular weight of 150 kDa, was substituted.

### Method:

1. Prepared GelMA/gelatin gels from GelMA (ALLEVI™ cat. no. GMA) and gelatin (ALLEVI™ cat. no. GEL) as follows:
   a. Group 1: 8% GelMA+4% gelatin (160 mg GelMA+40 mg gelatin)
   b. Group 2: 7% GelMA+3% gelatin (160 mg GelMA+80 mg gelatin)

2. Mixed 80 ug IgG with 400 uL GelMA+gelatin suspension (20 ug IgG/100 uL GelMA/gelatin). The same suspensions without IgG were prepared to use as controls.
3. Pipetted 4×100 uL onto the inside lid of 6 well plate.
4. Crosslinked suspensions using the blue light on the printer for 30 sec (80% intensity, distance from light to samples is ˜6 mm).
5. Transferred each gel to a 1.5 mL microcentrifuge tube.
6. Added1 mL 1×PBS release medium to each tube for release.
7. The release was carried out at 37C (37° C.) in a water bath in the dark to avoid photobleaching of the FITC tag.
8. At each predetermined timepoint, the 1×PBS release medium in each tube was removed and another fresh 1 ml PBS was added to the tube.
9. The predetermined timepoints were 3 h, 6 h, 12 h, 1 d, 2 d, 3 d, 5 d, 7 d, 10 d, 14 d, 21 d (h=hour(s); d=day(s)).
10. The in vitro sustained release study measured release of FITC-tagged mouse IgG from the GelMA/gelatin combination hydrogels into 1× phosphate buffered saline (PBS) release medium, as measured by flow cytometry.

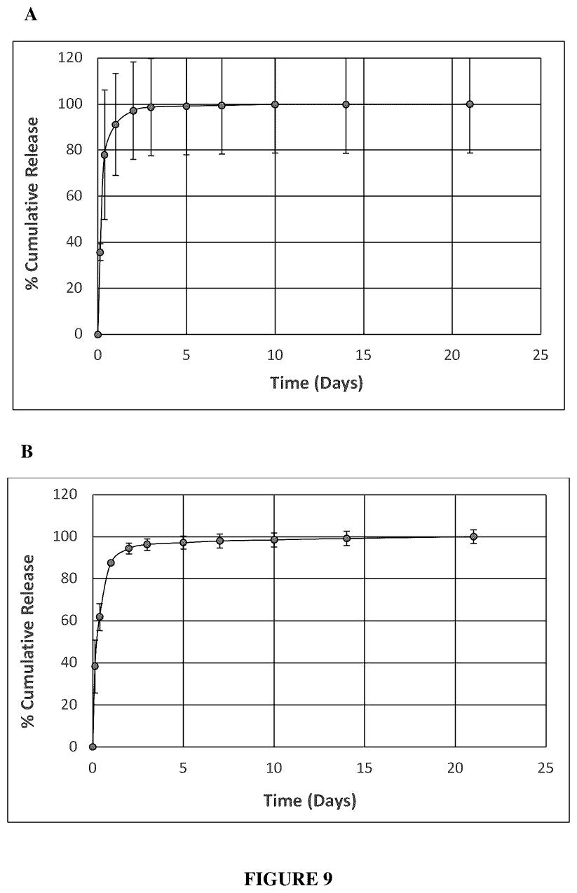

FIGS. 9A-9B are graphs depicting sustained release of fluorescently-tagged mouse immunoglobulin G (IgG) from collagen type I hydrogel compositions into 1× phosphate buffered saline (PBS), used as a release medium, at 37C (37° C.) in vitro. Compositions of 8% GelMA+4% gelatin and (FIG. 9A) and 7% GelMA+3% gelatin (FIG. 9B) were both prepared with 20 micrograms (μg, ug) FITC-tagged IgG per 100 microliters (μL, uL) composition. FIG. 9A shows the percentage of fluorescently tagged IgG release as a function of time from a hydrogel composition of 8% GelMA+4% gelatin. FIG. 9B shows the percentage of fluorescently tagged IgG release as a function of time from a hydrogel composition of 7% GelMA+3% gelatin.

### Example 19: In vitro Expansion of Lymph Cells in GelMA/Gelatin Combination Hydrogel

8% GelMA/4% gelatin suspension was prepared and FRC were formulated into the suspension and, following gelation, encapsulated into the hydrogel at an initial cell density of 10 M/mL. Live/Dead staining (Live=green, Dead=red) was performed. Cell growth was detected with LiveDead staining, namely, viable cells stained green (calcein [fluorexon, fluorescein complex]), whereas dead cells stained red (ethidium homodimer-1). Electron micrographs were taken at Day 1 and Day 7.

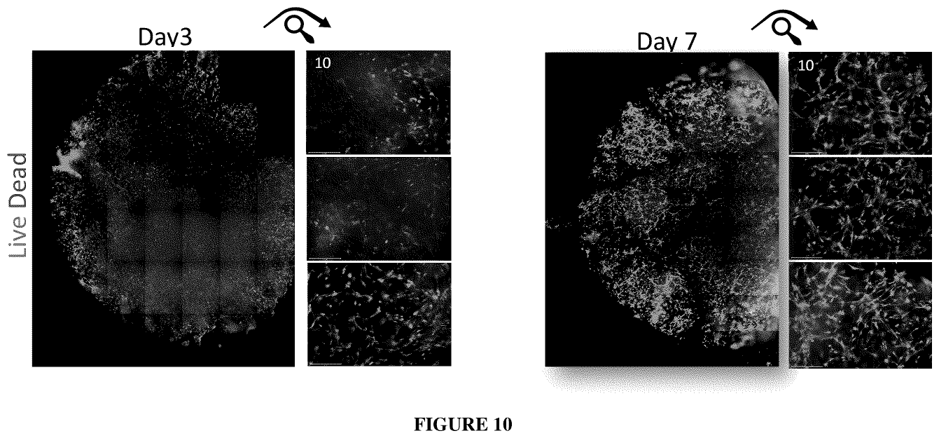

FIG. 10 shows groups of fluorescent images depicting the in vitro proliferation and spread of FRC encapsulated into a hydrogel composition (8% GelMA+4% gelatin) at an initial cell density of 10 M/mL. The images were taken at Day 3 (left set of panels) and Day 7 (right set of panels). The larger panel of each set is a view of combined 10× images while the three smaller panels of each set each depict a 10× magnification of the large panel. After 7 days of culture, stromal networks formed through branching and joining with other adjacent cell populations.

### Example 20: Subcutaneous Administration of Hydrogel Vaccine Adjacent to a Tumor in Vivo

Inoculating C57BL/6 inbred female mice with B16F10 melanoma cells: B16F10 melanoma cells were suspended in PBS at a concentration of 2×106 (2×10⁶) cells/mL at the final step. Immediately following suspension, two hundred thousand (2×105, 2×10⁵) B16F10 cells suspended in 0.1 mL PBS were injected subcutaneously in the right flank of each of 55 C57BL/6 inbred female mice.

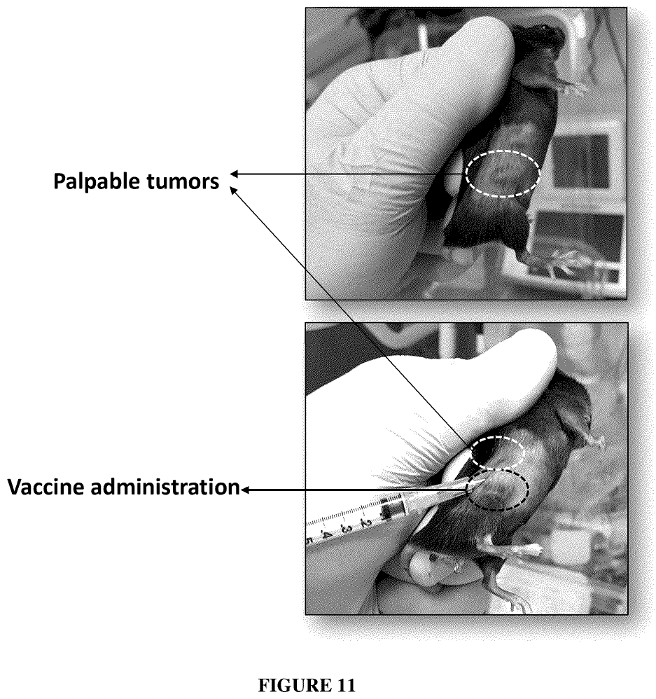

Measuring tumor size: When the tumors became palpable (FIG. 11, top), they were measured by a caliper in two dimensions in millimeter (mm), and the volume of each tumor was then calculated according to the following formula: Tumor Volume (mm3, mm³)=L×W²×0.5236, (L×W2×0.5236) (L=length, W=width).

Starting treatment: Tumors reached the expected size on Day 6 (closest to 50 mm3 [mm³]). Then they were randomly assigned into one of six groups (n=6-8). Each group received one regimen in pre-determined dose(s) and schedule(s) adjacent to a tumor (FIG. 11, bottom).

### Example 21: Hydrogel Vaccine Used to Form Lymphoid Tissue In Vivo

A vaccine polymer suspension was prepared with FRC cells, nivolumab, ipilimumab and cytokines (LT-alpha1-beta2+CCL20+CXCL13). The suspension was injected subcutaneously into a mouse.

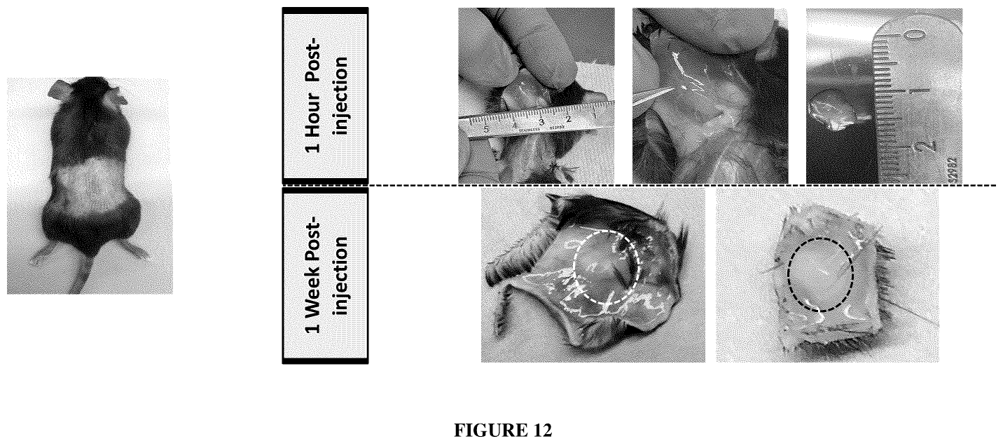

FIG. 12 is a series of photographs depicting lymphoid tissue formation in vivo. A C57BL/6 inbred female mouse is shaved in preparation for treatment (left), followed by subcutaneous injection of 4.5 mg/mL collagen type I solution. At one hour post-injection, the subcutaneous hydrogel formed is examined and measured (top right). At one week post-injection, the hydrogel still remains at the injection site indicating the hydrogel stability in mice (bottom right).

### Example 22: Stromal Scaffold Therapy for Melanoma In Vivo

C57BL/6 inbred female mice were transfected with mouse B16F10 melanoma cells and divided into six groups.

A polymer suspension was prepared as described herein.

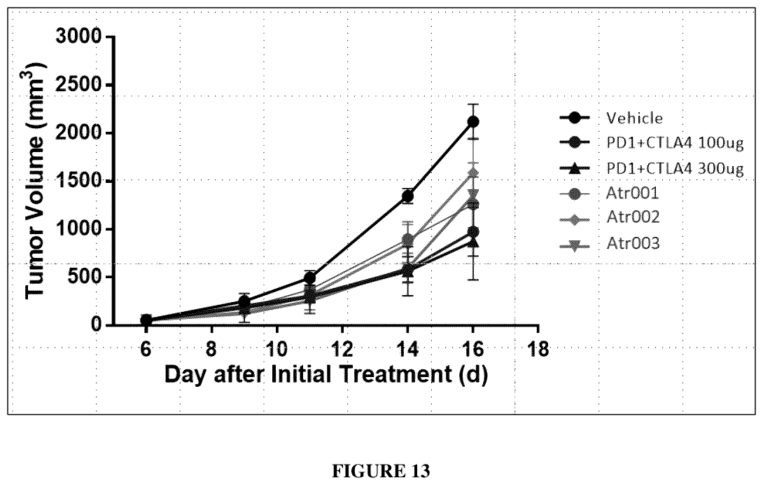

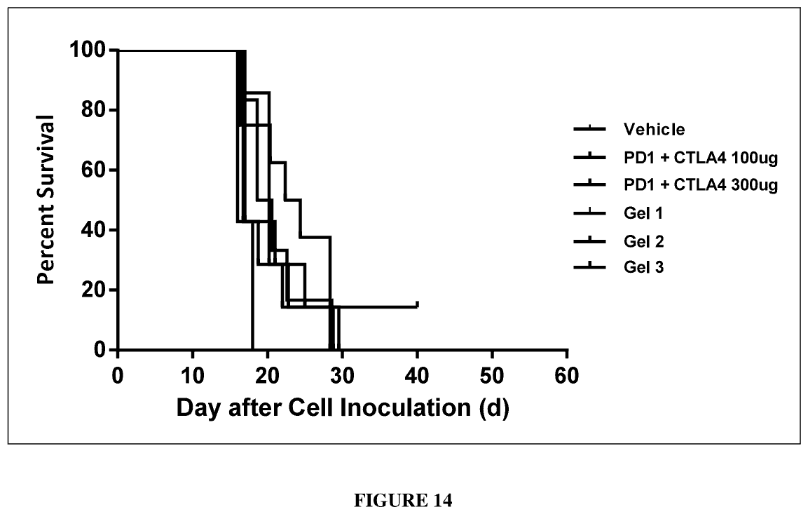

FIG. 13 is a graph depicting efficacy of the hydrogel stromal scaffold as combination therapy with nivolumab and ipilimumab with respect to tumor volume (mm2 [mm²]) over time (days after initial treatment). C57BL/6 inbred female mice were transfected with mouse B16F10 melanoma cells. Three sets of mice were treated as follows: Seven (7) mice were injected with Gel Atr001 (comprising anti-mouse PD-1 antibody [300 micrograms/mouse]+anti-mouse CTLA-4 antibody [300 micrograms/mouse]) on Day 6. Seven (7) mice were injected with Gel Atr002 (lymphoid stromal cell+chemokine+cytokine) injected subcutaneously on Day 6. Seven (7) mice were injected with Gel Atr003 (comprising anti-mouse PD-1 antibody (300 micrograms/mouse)+anti-mouse CTLA-4 antibody (300 micrograms/mouse)+stomal cell lymphoid+chemokine+cytokine) at Day 6. As a negative control, 7 mice were injected with one dose of vehicle (saline) on Day 6. As positive controls, seven (7) mice received one dose of a combination of anti-mouse PD-1 antibody (100 micrograms/mouse)+anti-mouse CTLA-4 antibody (100 micrograms/mouse) injected at Day 6, Day 9, and Day 12, while eight (8) mice received a single dose of a combination of anti-mouse PD-1 antibody (300 micrograms/mouse)+anti-mouse CTLA-4 antibody (300 micrograms/mouse) injected at Day 6.

FIG. 14 is a graph further depicting efficacy of the hydrogel stromal scaffold as combination therapy with nivolumab and ipilimumab with respect to survival rate (percent survival; %) over time (days after cell inoculation [d]). C57BL/6 inbred female mice were transfected with mouse B16F10 melanoma cells. Three sets of mice were treated as follows: Seven (7) mice were injected with Gel Atr001 (comprising anti-mouse PD-1 antibody [300 micrograms/mouse]+anti-mouse CTLA-4 antibody [300 micrograms/mouse]) on Day 6. Seven (7) mice were injected with Gel Atr002 (lymphoid stromal cell+chemokine+cytokine) injected subcutaneously on Day 6. Seven (7) mice were injected with Gel Atr003 (comprising anti-mouse PD-1 antibody (300 micrograms/mouse)+anti-mouse CTLA-4 antibody (300 micrograms/mouse)+stomal cell lymphoid+chemokine+cytokine) at Day 6. As a negative control, 7 mice were injected with one dose of vehicle (saline) on Day 6. As positive controls, seven (7) mice received one dose of a combination of anti-mouse PD-1 antibody (100 micrograms/mouse)+anti-mouse CTLA-4 antibody (100 micrograms/mouse) injected at Day 6, Day 9, and Day 12, while eight (8) mice received a single dose of a combination of anti-mouse PD-1 antibody (300 micrograms/mouse)+anti-mouse CTLA-4 antibody (300 micrograms/mouse) injected at Day 6.

### Example 23: Anti-tumor Efficacy of Checkpoint Inhibitor Anti-Cancer Drugs Loaded in Artificial Lymph Nodes in Treating Mouse Melanoma In Vivo

The anti-tumor efficacy of checkpoint inhibitor anti-cancer drugs loaded in artificial lymph nodes (LN) in B16F10 melanoma was investigated in vivo in mice. Six (6) groups of seven (7) mice/group were utilized for a total of 42 mice. The date of B16F10 implantation was Day 0. The study design is summarized in Table 2.

TABLE 2
Study Design Using Six (6) Groups of Seven (7) Mice/Group.

| | | | Dose (mpk) | Route |
|---|---|---|---|---|
| | | | anti-CTLA-4 + | and |
| Group | N | Treatment | anti-PD-1 | Schedule |
| 1 | 7 | Saline | — | SC, Days 6, |
| | | | | 9, 12 |
| 2 | 7 | anti-CTLA-4 + anti-PD-1 | 100 + 100, SC | SC, Days 6, |
| | | | | 9, 12 |
| 3 | 7 | anti-CTLA-4 + anti-PD-1 | 300 + 300, SC | SC, Day 6 |
| 4 | 7 | Collagen type I + | 300 + 300, SC | SC, Day 6 |
| | | anti-CTLA-4 + anti-PD-1 | | |
| 5 | 7 | Collagen type I + stromal | 300 + 300, SC | SC, Day 6 |
| | | cells + LT-alpha1-beta2 + | | |
| | | CCL20 + CXCL13 | | |
| 6 | 7 | Collagen type I + stromal | 300 + 300, SC | SC, Day 6 |
| | | cells + LT-alpha1-beta2 + | | |
| | | CCL20 + CXCL13 + anti- | | |
| | | CTLA-4 + anti-PD-1 | | |

### Materials:

Collagen (HUMABIOLOGICS™, HumaDerm-Human Skin Collagen
Type I, Lyophilized 100 mg): 14 mg/mL
Anti-CTLA-4 (BIOXCELL™, cat. no. BE0131, Clone #9H10): 8.35 mg/mL
Anti-PD-1 (BIOXCELL™, cat. no. BE0146, Clone #RMP1-14): 8.11 mg/mL
LT-alpha1-beta2 (R&D SYSTEMS™, cat. no. 9968-LYICF): reconstituted with PBS to obtain 100 μg/mL
CCL20 (PEPROTECH™, cat. no. 250-27, 100 ug): reconstituted with 100 uL water to obtain 1 mg/mL

### Concentration:

1. Collagen type I: 7.4 mg/mL
2. FRC: cell density is 20 M/mL
   Only group 5 and 6 need FRC, so N=20: 20×200 ul=4000 uL (4 mL). Cell density was 20 M/mL, so 80M FRC were needed.
3. LT-alpha1-beta2, CXCL13, CCL20: 500 ng for each injection for each cytokine. Only groups 5 and 6 need these reagents, so N=20.
Total 20×500 ng=10 ug, so each group needed 5 μg of each cytokine
4. Drugs (anti-CTLA-4 and anti-PD-1): 300 ug for each injection for groups 2, 3, 4, and 6. N=10×4 groups=40 injections.
Basically, each injection was 300 ug. so total: 40×300 ug=12 mg.
Therefore, each group needed 3 mg of each drug.

Table 3 summarizes the above amounts.

TABLE 3
Preparation of Compositions for Injections into Mice.

| | | 10X | NaOH | NaOH | | | Anti | Anti | 1X | | | | |
|---|---|---|---|---|---|---|---|---|---|---|---|---|---|
| | Collagen | PBS | 0.1N | 1N | Water | FRC | CTLA4 | PD1 | PBS | CCL20 | CXCL13 | LTα1β2 | Total |
| Groups | μl | μl | μl | μg | μl | (million) | μl | μl | μl | μl | μl | μl | μl |
| 1 | — | — | — | — | — | — | — | — | — | — | — | — | — |
| 2-1 | — | — | — | — | — | — | 120 | 123 | 1758 | — | — | — | 2000 |
| 2-2 | — | — | — | — | — | — | 120 | 123 | 1758 | — | — | — | 2000 |
| 2-3 | — | — | — | — | — | — | 120 | 123 | 1758 | — | — | — | 2000 |
| 3 | — | — | — | — | — | — | 359 | 370 | 1271 | — | — | — | 2000 |
| 4 | 1057 | 200 | — | 22 | — | — | 359 | 370 | — | — | — | — | 2008 |
| 5 | 1057 | 200 | 220 | — | 453 | 40M | — | — | — | 5 | 5 | 50 | 2000 |
| 6 | 1144 | 200 | — | 22 | — | 40M | 359 | 370 | — | 5 | 5 | 50 | 2155 |

### Methods:

The injections into the mice were performed using a 1 mL syringe with a 26G needle. After loading, the syringe was kept on ice to prevent viscosity increase. The solution or suspension was easily injected into mice using the 1 mL syringe equipped with the 26G needle.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of using a hydrogel vaccine to form an expansion of a lymphoid network in a subject in need thereof, the method comprising:
(a) administering to the subject a thermo-responsive polymer suspension composition comprising: a natural polymer, the natural polymer comprising collagen and one or more cells;
(b) gelling the polymer suspension composition in vivo to form a hydrogel composition scaffold; and
(c) expanding the population of the one or more cells to form an expansion of the lymphoid network.

2. The method of claim 1, wherein the expansion of the lymphoid network comprises growth or development of lymphoid tissue.

3. The method of claim 1, wherein the expansion of the lymphoid network comprises growth or development of a high endothelial vacuole (HEV).

4. The method of claim 1, wherein the collagen comprises gelatin methacryloyl (GelMA) or gelatin.

5. The method of claim 1, the polymer suspension composition further comprising a pharmaceutical or biopharmaceutical composition, a modulating agent, a targeting agent, a stabilizing agent, or a combination of any of these.

6. The method of claim 1, the polymer suspension composition further comprising one or more drugs and the method further comprising controlled release of the one or more drugs.

7. The method of claim 1, the polymer suspension composition comprises a pharmaceutical or biopharmaceutical composition, a modulating agent, or combination thereof that inhibits the programmed cell death process.

8. The method of claim 1, the polymer suspension composition further comprising a cytokine, a chemokine, or an antibody, wherein said cytokine, said chemokine, or said antibody comprises an immune checkpoint inhibitor or an anti-receptor activator of nuclear factor kappa-B ligand (anti-RANKL) agent.

9. The method of claim 1, the polymer suspension composition further comprising a chemokine, wherein said chemokine comprises chemokine (C-C motif) ligand 20 (CCL20) or chemokine (C-X-C motif) ligand 13 (CXCL13).

10. The method of claim 1, wherein the subject has a cancer or a tumor and wherein the method further comprises treating, inhibiting, ameliorating, or alleviating the cancer or the tumor or reducing or inhibiting growth, spread, or metastasis of the cancer or the tumor in the subject.

11. The method of claim 10, wherein the hydrogel vaccine is a cancer vaccine.

12. The method of claim 10, wherein the cancer is a melanoma, a fibrosarcoma, a lymphoma, a breast tumor, a pancreatic tumor, or a metastasis of any of these.

13. The method of claim 10, wherein the tumor is a fibroma.

14. The method of claim 1, the administration of the polymer suspension composition comprises subcutaneous (SC) injection, intraperitoneal (IP) injection, intravenous injection (IV), intra-tumor injection, intra-lymph node injection, or tumor-adjacent injection in the subject.

15. The method of claim 1, wherein the subject is a human or non-human mammal or a bird.

16. The method of claim 1, wherein the gelling of the polymer suspension composition is thermo-responsive.

17. The method of claim 16, wherein the subject is a mammal and wherein gelation of the polymer suspension occurs from about 35.0C (35.0° C.) to about 41.0C (41.0° C.).

18. The method of claim 1, wherein the hydrogel composition has a sustained-release (SR) dosage or an extended-release (ER) dosage.

19. The method of claim 18, wherein the SR dosage or the ER dosage extends at least about 7 days.

20. The method of claim 1, the polymer suspension composition comprising about 1 mg/mL collagen to about 30 mg/mL collagen.

21. The method of claim 1, the one or more cells comprising one or more stromal cells, one or more adipose cells, or one or more stem cells.

22. The method of claim 21, the one or more stromal cells comprising one or more fibroblast reticular cells (FRC), one or more follicular dendritic cells (FDC), one or more marginal reticular cells (MRC), one or more lymphatic endothelial cells (LEC), one or more high endothelial cells (HEC), one or more alpha-7 integrin pericytes (AIP), or a combination of any of these.

23. The method of claim 21, the one or more stromal cells comprising one or more stem cells, one or more differentiated stem cells, one or more modified stem cells, or one or more modified adipose cells, one or more mesenterial stem cells.

24. The method of claim 21, the one or more cells comprising one or more non-immunogenic cells.

25. The method of claim 21, the one or more cells comprising one or more cells collected from the subject in need prior to step (a).

26. The method of claim 5, the polymer suspension composition comprising a pharmaceutical or biopharmaceutical composition, wherein the pharmaceutical or biopharmaceutical composition inhibits, ameliorates, or alleviates a cancer or growth thereof or a tumor or growth thereof or reduces or inhibits a metastasis of a cancer.

27. The method of claim 5, the polymer suspension composition comprising a modulating agent.

28. The method of claim 27, the modulating agent comprising an immunomodulating agent.

29. The method of claim 27, the modulating agent comprising a cytokine or a chemokine, an antibody or an antigen-binding domain, a nucleic acid, or a combination of any of these.

30. The method of claim 29, the cytokine comprising lymphotoxin-alpha (LT-alpha), lymphotoxin-beta (LT-beta), lyphotoxin-alpha1-beta2 heterotrimer (LT-alpha1-beta2), lymphotoxin-alpha2-beta1 heterotrimer (LT-alpha2-beta1), or a combination of any of these.

31. The method of claim 29, the antibody or antigen-binding domain comprising a monoclonal antibody (mAb), a single chain variable fragment (scFv), a bispecific single chain variable fragment (diabody), a trispecific single chain variable fragment (triabody), a bispecific antibody, an antibody-drug conjugate (ADC), or a combination of any of these.

32. The method of claim 29, the antibody or antigen-binding domain comprising an anti-cytotoxic T-lymphocyte-associated protein 5 (anti-CTLA-4) antibody or antigen-binding domain, an anti-programmed cell death protein 1 (anti-PD-1) antibody or antigen-binding domain, or an anti-programmed death-ligand 1 (anti-PD-L1) antibody or antigen-binding domain, an anti-receptor activator of nuclear factor kappa-B ligand (anti-RANKL) antibody, or a combination of any of these.

33. The method of claim 29, the nucleic acid comprising a deoxyribonucleic acid (DNA) or a ribonucleic acid (RNA).

34. The method of claim 33, the DNA comprising an antisense DNA or a portion thereof, a complementary DNA (cDNA) or a portion thereof, or a genomic DNA or a portion thereof.

35. The method of claim 33, the RNA comprising a small interfering RNA (siRNA), microRNA, or messenger RNA (mRNA) or a portion thereof.

36. The method of claim 5, the polymer suspension composition comprising a targeting agent.

37. The method of claim 36, the targeting agent comprising a cytokine or a chemokine, an antibody or an antigen-binding domain, a nucleic acid, or a combination of any of these.

38. The method of claim 37, the antibody or antigen-binding domain comprising a monoclonal antibody (mAb), a single chain variable fragment (scFv), a bispecific single chain variable fragment (diabody), a trispecific single chain variable fragment (triabody), a bispecific antibody, an antibody-drug conjugate (ADC), or a combination of any of these.

39. The method of claim 38, the antibody or antigen-binding domain comprising an anti-cytotoxic T-lymphocyte-associated protein 5 (anti-CTLA-4) antibody or antigen-binding domain, an anti-programmed cell death protein 1 (anti-PD-1) antibody or antigen-binding domain, or an anti-programmed death-ligand 1 (anti-PD-L1) antibody or antigen-binding domain, or a combination of any of these.

40. The method of claim 37, the nucleic acid comprising a deoxyribonucleic acid (DNA) or a ribonucleic acid (RNA).

41. The method of claim 40, the DNA comprising an antisense DNA or a portion thereof, a complementary DNA (cDNA) or a portion thereof, or a genomic DNA or a portion thereof.

42. The method of claim 40, the RNA comprising a small interfering RNA (siRNA), microRNA, or messenger RNA (mRNA) or a portion thereof.

43. The method of claim 5, the natural polymer further comprising gelatin, fibrin, chitosan, cellulose, poly (lactic-co-glycolic acid), agarose, methylcellulose, hyaluronan, or an elastin-like polypeptide.

44. A thermo-responsive hydrogel vaccine comprising a thermo-responsive polymer composition, the thermo-responsive polymer suspension composition comprising:
(a) a natural polymer, the natural polymer comprising collagen; and
(b) one or more cells.

45. The thermo-responsive hydrogel vaccine of claim 44, the thermo-responsive polymer suspension composition comprising about 1 mg/mL collagen to about 30 mg/mL collagen.

46. The thermo-responsive hydrogel vaccine of claim 44, wherein, prior to gelation, the thermo-responsive polymer suspension composition is injectable.

47. The thermo-responsive hydrogel vaccine of claim 44, wherein gelation of the thermo-responsive polymer suspension composition is temperature-dependent.

48. The thermo-responsive hydrogel vaccine of claim 44, wherein the collagen comprises gelatin methacryloyl (GelMA) or gelatin.

49. The thermo-responsive hydrogel vaccine of claim 44, the one or more cells comprising a stromal cell, an adipose cell, or a stem cell.

50. The thermo-responsive hydrogel vaccine of claim 49, the stromal cell comprising a fibroblast reticular cell (FRC), a follicular dendritic cell (FDC), a marginal reticular cell (MRC), a lymphatic endothelial cell (LEC), a high endothelial cell (HEC), an alpha-7 integrin pericyte (AIP), or a combination of any of these.

51. The thermo-responsive hydrogel vaccine of claim 44, the one or more cells comprising a non-immunogenic cell.

52. The thermo-responsive hydrogel of claim 44, the one or more cells comprising an autologous cell.

53. The thermo-responsive hydrogel vaccine of claim 44, the thermo-responsive polymer suspension composition further comprising a pharmaceutical or biopharmaceutical composition, a modulating agent, a targeting agent, a stabilizing agent, one or more drugs, or a combination of any of these.

54. The thermo-responsive hydrogel vaccine of claim 53, the thermo-responsive polymer suspension composition comprising a pharmaceutical or biopharmaceutical composition.

55. The thermo-responsive hydrogel vaccine of claim 54, wherein the pharmaceutical or biopharmaceutical composition inhibits, ameliorates, or alleviates a cancer or growth thereof or a tumor or growth thereof, or reduces or inhibits a growth or a metastasis of a cancer or a tumor.

56. The thermo-responsive hydrogel vaccine of claim 44, wherein the thermo-responsive hydrogel vaccine is a cancer vaccine.

57. The thermo-responsive hydrogel vaccine of claim 49, the thermo-responsive polymer suspension composition comprising a modulating agent.

58. The thermo-responsive hydrogel vaccine of claim 57, the modulating agent comprising an immunomodulating agent.

59. The thermo-responsive hydrogel vaccine of claim 57, the modulating agent comprising a cytokine or a chemokine, an antibody or an antigen-binding domain, a nucleic acid, a or a combination of any of these.

60. The thermo-responsive hydrogel vaccine of claim 59, the cytokine comprising lymphotoxin-alpha (LT-alpha), lymphotoxin-beta (LT-beta), lyphotoxin-alpha1-beta2 heterotrimer (LT-alpha1-beta2), lymphotoxin-alpha2-beta1 heterotrimer (LT-alpha2-beta1), or a combination of any of these.

61. The thermo-responsive hydrogel vaccine of claim 59, the chemokine comprising chemokine (C-C motif) ligand 20 (CCL20) or chemokine (C-X-C motif) ligand 13 (CXCL13).

62. The thermo-responsive hydrogel vaccine of claim 59, the antibody or antigen-binding domain comprising a monoclonal antibody (mAb), a single chain variable fragment (scFv), a bispecific single chain variable fragment (diabody), a trispecific single chain variable fragment (triabody), a bispecific antibody, an antibody-drug conjugate (ADC), or a combination of any of these.

63. The thermo-responsive hydrogel vaccine of claim 59, the antibody or antigen-binding domain comprising an anti-cytotoxic T-lymphocyte-associated protein 5 (anti-CTLA-4) antibody or antigen-binding domain, an anti-programmed cell death protein 1 (anti-PD-1) antibody or antigen-binding domain, or an anti-programmed death-ligand 1 (anti-PD-L1) antibody or antigen-binding domain, an anti-receptor activator of nuclear factor kappa-B ligand (anti-RANKL) antibody or a combination of any of these.

64. The thermo-responsive hydrogel vaccine of claim 59, the nucleic acid comprising a deoxyribonucleic acid (DNA) or a ribonucleic acid (RNA).

65. The thermo-responsive hydrogel vaccine of claim 64, the DNA comprising an antisense DNA or a portion thereof, a complementary DNA (cDNA) or a portion thereof, or a genomic DNA or a portion thereof.

66. The thermo-responsive hydrogel vaccine of claim 64, the RNA comprising a small interfering RNA (siRNA), microRNA, or messenger RNA (mRNA) or a portion thereof.

67. The thermo-responsive hydrogel vaccine of claim 44, the thermo-responsive polymer suspension composition comprising a targeting agent.

68. The thermo-responsive hydrogel vaccine of claim 67, the targeting agent comprising a cytokine or a chemokine, an antibody or an antigen-binding domain, a nucleic acid, or a combination of any of these.

69. The thermo-responsive hydrogel vaccine of claim 68, the antibody or antigen-binding domain comprising a monoclonal antibody (mAb), a single chain variable fragment (scFv), a bispecific single chain variable fragment (diabody), a trispecific single chain variable fragment (triabody), a bispecific antibody, an antibody-drug conjugate (ADC), or a combination of any of these.

70. The thermo-responsive hydrogel vaccine of claim 68, the antibody or antigen-binding domain comprising an anti-cytotoxic T-lymphocyte-associated protein 5 (anti-CTLA-4) antibody or antigen-binding domain, an anti-programmed cell death protein 1 (anti-PD-1) antibody or antigen-binding domain, or an anti-programmed death-ligand 1 (anti-PD-L1) antibody an anti-RANKL antibody or antigen-binding domain, or a combination of any of these.

71. The thermo-responsive hydrogel vaccine of claim 67, the nucleic acid comprising a deoxyribonucleic acid (DNA) or a ribonucleic acid (RNA).

72. The thermo-responsive hydrogel vaccine of claim 71, the DNA comprising an antisense DNA or a portion thereof, a complementary DNA (cDNA) or a portion thereof, or a genomic DNA or a portion thereof.

73. The thermo-responsive hydrogel vaccine of claim 72, the RNA comprising a small interfering RNA (siRNA), microRNA, or messenger RNA (mRNA) or a portion thereof.

74. The thermo-responsive hydrogel vaccine of claim 44, the natural polymer further comprising gelatin, fibrin, chitosan, cellulose, poly (lactic-co-glycolic acid), agarose, methylcellulose, hyaluronan, or an elastin-like polypeptide.

75. A method of making a thermo-responsive hydrogel vaccine, the method comprising:
(a) preparing, at a temperature at or below about 4C (4° C.), a collagen polymer suspension in a physiologically acceptable buffer;
(b) mixing one or more cells into the collagen polymer suspension; and
(c) placing the collagen suspension into an environment having a temperature of about 35.0C (35.0° C.) to about 41.0C (41.0° C.) for gelation of hydrogel composition.

76. The method of claim 75, step (b) further comprising mixing a pharmaceutical or biopharmaceutical composition, a modulating agent, a targeting agent, a stabilizing agent, or a combination of any of these with the one or more cells into the collagen polymer suspension.
